(19) United States
(12) Patent Application Publication
(10) Pub. No.: US 2022/0281888 A1
(43) Pub. Date: Sep. 8, 2022

(54) BICYCLIC CARBOXYLATES AS MODULATORS OF TRANSPORTERS AND USES THEREOF

(71) Applicant: NIROGY THERAPEUTICS, INC., Framingham, MA (US)

(72) Inventors: Vincent SANDANAYAKA, Northborough, MA (US); Gregory GORECZNY, Cambridge, MA (US)

(21) Appl. No.: 17/638,088

(22) PCT Filed: Sep. 24, 2020
(86) PCT No.: PCT/US2020/052413

§ 371 Date: Feb. 24, 2022

Related U.S. Application Data
(60) Provisional application No. 62/905,606, filed on Sep. 25, 2019.

(51) Int. Cl. C07D 495/04

(52) U.S. Cl. CPC C07D 495/04

(57) ABSTRACT

The present invention generally relates to the field of transporter modulators, e.g., monocarboxylate transporter inhibitors, and more particularly to new bicyclic enone carboxylate compounds, the synthesis and use of these compounds and their pharmaceutical compositions, e.g., in the treatment, modulation and/or prevention of physiological conditions associated with monocarboxylate transporter activity such as in treating cancer and other neoplastic disorders, tissue and organ transplant rejection.

### CROSS-REFERENCES TO RELATED APPLICATIONS

This application claims priority to U.S. Provisional Application No. 62/905,606 filed Sep. 25, 2019, which is incorporated herein in its entirety for all purpose.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### FIELD OF THE INVENTION

The present invention relates to compounds useful as transporter modulators. The invention also provides pharmaceutically acceptable compositions comprising compounds of the present invention and methods of using said compositions in the treatment of various disorders.

### BACKGROUND OF THE INVENTION

It has been well demonstrated that tumors display altered cellular metabolism, in which cancer cells exhibit high rate of glucose consumption compared to the untransformed normal cells. Tumors contain well oxygenated (aerobic), and poorly oxygenated (hypoxic) regions. Compared to normal cells, some cancer cells are heavily dependent upon either aerobic glycolysis (Warburg effect, 1956) or anaerobic glycolysis (especially in hypoxic regions) for energy (ATP) production while maintaining a certain level of oxidative phosphorylation. This glycolytic switch by highly proliferating and hypoxic cancer cells provides the energy and biosynthetic needs for cancer cell survival. To maintain this metabolic phenotype, cancer cells up regulate a series of proteins, including glycolytic enzymes and pH regulators; monocarboxylate transporters (MCTs) that will facilitate the efflux of lactate co-transported with a proton. This fundamental difference between normal cells and cancer cells has not been previously applied to cancer therapy.

MCTs mediate influx and efflux of monocarboxylates such as lactate, pyruvate, ketone bodies (acetoacetate and beta-hydroxybutyrate) across cell membranes. These monocarboxylates play essential roles in carbohydrate, amino acid, and fat metabolism in mammalian cells, and must be rapidly transported across plasma membrane of cells. MCTs catalyse the transport of these solutes via a facilitative diffusion mechanism that requires co-transport of protons. Monocarboxylates such as lactate, pyruvate, and ketone bodies play a central role in cellular metabolism and metabolic communications among tissues. Lactate is the end product of aerobic glycolysis. Lactate has recently emerged as a critical regulator of cancer development, invasion, and metastasis. Tumor lactate levels correlate well with metastasis, tumor recurrence, and poor prognosis (J.Clin.Invest 2013).

MCTs are 12-span transmembrane proteins with N- and C-terminus in cytosolic domain, and are members of solute carrier SLC16A gene family. MCT family contains 14 members, and so far MCT1, MCT2, MCT3, and MCT4 are well characterized [Biochemical Journal (1999), 343:281-299].

Malignant tumors contain aerobic and hypoxic regions, and the hypoxia increases the risk of cancer invasion and metastasis. Tumor hypoxia leads to treatment failure, relapse, and patient mortality as these hypoxic cells are generally resistant to standard chemo- and radiation therapy. In regions of hypoxia, cancer cells metabolize glucose into lactate whereas nearby aerobic cancer cells take up this lactate via the MCT1 for oxidative phosphorylation (OXPHOS). Under hypoxic conditions, cancer cells up regulate glucose transporters and consume large quantities of glucose. Cancer cells also up regulate glycolytic enzymes and convert glucose into lactate, which is then efflux out of cell via MCT4. The nearby aerobic cancer cells take up this lactate via MCT1 for energy generation through OXPHOS. Thus, the limited glucose availability to the tumor is used most efficiently via synergistic metabolic symbiosis. This utilization of lactate as an energy substitute for survival prevents the aerobic cells from consuming large quantities of glucose.

### BRIEF SUMMARY OF THE INVENTION

In a first aspect, the present invention provides a compound represented by formula (I):

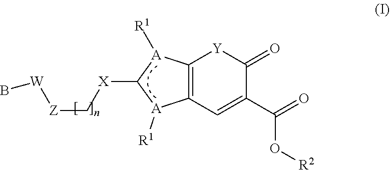

or a pharmaceutically acceptable salt thereof, wherein subscript n, each A, B, W, X, Y, Z,

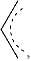

each R¹, and R² are provided herein.

In a second aspect, the present invention provides a pharmaceutical composition including a compound of formula (I) or a compound described herein, or a pharmaceutically acceptable salt thereof, together with a pharmaceutically acceptable carrier.

In a third aspect, the present invention provides a method for modulating monocarboxylate transport. The method includes contacting a monocarboxylate transport protein with a therapeutically effective amount of a compound of formula (I), a compound, or a composition thereof described herein.

In a fourth aspect, the present invention provides a method for treating a disorder associated with monocarboxylate transport. The method includes administering a therapeutically effective amount of a compound a compound of formula (I) a compound of formula (I), a compound, or a composition thereof described herein.

In a fifth aspect, the present invention provides a process for preparing a compound of formula (VIII):

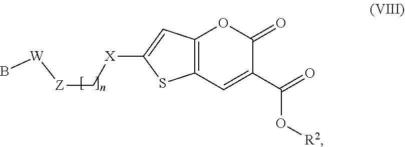

or a pharmaceutically acceptable salt thereof, wherein subscript n, B, W, X, Z, and R² are provided herein.

### BRIEF DESCRIPTION OF THE DRAWINGS

FIG. 1 shows an exemplary general method for preparing compounds of formula (I) according to Scheme 1.

FIG. 2 shows an exemplary general method for preparing certain bicyclic enone carboxylic acid compounds according to Scheme 2.

FIG. 3 shows an exemplary general method for preparing compounds of formula (I) and certain bicyclic enone carboxylic acid compounds according to Scheme 3.

FIG. 4 shows the preparation of core structures in formula (I) according to Scheme 4.

### DETAILED DESCRIPTION OF THE INVENTION

### I. General

The present invention provide compounds of formula (I) and related compounds as transporter modulators, for example, monocarboxylate transporter inhibitors. In particular, the present invention provide novel bicyclic enone carboxylate compounds and the preparation thereof, and use of these compounds and their pharmaceutical compositions in the treatment, modulation and/or prevention of physiological conditions associated with monocarboxylate transporter activity such as in treating cancer and other neoplastic disorders, tissue and organ transplant rejection.

### II. Definitions

As used herein, the following definitions shall apply unless otherwise indicated. For purposes of this invention, the chemical elements are identified in accordance with the Periodic Table of the Elements CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

Compounds of the present invention may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers or enantiomers, with all possible isomers and mixtures thereof, including optical isomers, being included in the present invention.

Generally, reference to a certain element such as hydrogen or H is meant (if appropriate) to include all isotopes of that element, for example, deuterium and tritium for hydrogen.

The term “alkyl” as used herein means a straight- or branched-chain hydrocarbon having from one to eight carbon atoms, and includes, for example, and without being limited thereto, methyl, ethyl, propyl, isopropyl, t-butyl and the like. Substituted alkyl includes, for example, and without being limited thereto, haloalkyl, hydroxyalkyl, cyanoalkyl, and the like. This is applied to any of the groups mentioned herein, such as substituted “alkenyl”, “alkynyl”, “aryl”, etc.

The term “alkenyl” as used herein means a straight- or branched-chain aliphatic hydrocarbon having at least one double bond. The alkene may have from two to eight carbon atoms, and includes, for example, and without being limited thereto, ethenyl, 1-propenyl, 1-butenyl and the like. The term “alkenyl” encompasses radicals having “cis” and “trans” orientations, or alternatively, “E” and “Z” orientations.

The term “cycloalkyl” as used herein means an aliphatic carbocyclic system (which may be unsaturated) containing one or more rings wherein such rings may be attached together in a pendent manner or may be fused. In one aspect, the ring(s) may have from three to seven carbon atoms, and includes, for example, and without being limited thereto, cyclopropyl, cyclohexyl, cyclohexenyl and the like. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl.

The term “heterocycloalkyl” as used herein means a heterocyclic system (which may be unsaturated) having at least one heteroatom selected from N, S and/or O and containing one or more rings wherein such rings may be attached together in a pendent manner or may be fused. In one aspect, the ring(s) may have a three- to seven-membered cyclic group and includes, for example, and without being limited thereto, piperidinyl, piperazinyl, pyrrolidinyl, tetrahydrofuranyl, tetrahydropyranyl and the like.

The term “heteroatom” means one or more of oxygen, sulfur, nitrogen, phosphorus, or silicon.

The term “unsaturated”, as used herein, means that a moiety has one or more units of unsaturation.

The term “alkoxy” as used herein means a straight- or branched-chain oxygen-containing hydrocarbon; in one aspect, having from one to eight carbon atoms and includes, for example, and without being limited thereto, methoxy, ethoxy, propyloxy, isopropyloxy, t-butoxy and the like.

The term “halo” or “halogen” includes, for example, and without being limited thereto, fluoro, chloro, bromo, and iodo, in both radioactive and non-radioactive forms.

“Haloalkyl” refers to alkyl, as defined above, where some or all of the hydrogen atoms are replaced with halogen atoms. As for alkyl group, haloalkyl groups can have any suitable number of carbon atoms, such as C₁-C₆. For example, haloalkyl includes trifluoromethyl, fluoromethyl, 2,2,2-trifluoroethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁-C₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

The term “alkylene” as used herein means a difunctional branched or unbranched saturated hydrocarbon; in one aspect, having one to eight carbon atoms, and includes, for example, and without being limited thereto, methylene, ethylene, n-propylene, n-butylene and the like.

The term “aryl”, alone or in combination, as used herein means a carbocyclic aromatic system containing one or more rings. In particular embodiments, aryl is one, two or three rings. In one aspect, the aryl has five to twelve ring atoms. The term “aryl” encompasses aromatic radicals such as phenyl, naphthyl, tetrahydronaphthyl, indanyl, biphenyl, phenanthryl, anthryl or acenaphthyl. The “aryl” group may have 1 to 4 substituents such as lower alkyl, hydroxyl, halo, haloalkyl, nitro, cyano, alkoxy, lower alkylamino and the like.

The term “heteroaryl”, alone or in combination, as used herein means an aromatic system having at least one heteroatom selected from N, S and/or O and containing one or more rings. In particular embodiments, heteroaryl is one, two or three rings. In one aspect, the heteroaryl has five to twelve ring atoms. The term “heteroaryl” encompasses heteroaromatic groups such as triazolyl, imidazolyl, pyrrolyl, tetrazolyl, pyridyl, indolyl, furyl, benzofuryl, thienyl, benzothienyl, quinolyl, oxazolyl, thiazolyl and the like. The “heteroaryl” group may have 1 to 4 substituents such as lower alkyl, hydroxyl, halo, haloalkyl, nitro, cyano, alkoxy, lower alkylamino and the like.

It is understood that substituents and substitution patterns on the compounds of the invention may be selected by one of ordinary skill in the art to provide compounds that are chemically stable and that can be readily synthesized by techniques known in the art, as well as those methods set forth below. If a substituent is itself substituted with more than one group, it is understood that these multiple groups may be on the same carbon or on different carbons, as long as a stable structure results.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{º}; —(CH₂)₀₋₄R^{º}; —O(CH₂)₀₋₄R^{º}, —O—(CH₂)₀₋₄C(O)OR^{º}; —(CH₂)₀₋₄CH(OR^{º})₂; —(CH₂)₀₋₄SR^{º}; —(CH₂)₀₋₄Ph, which may be substituted with R^{º}; —(CH₂)₀₋₄O(CH₂)₀₋₁Ph which may be substituted with R^{º}; —CH═CHPh, which may be substituted with R^{º}; —(CH₂)₀₋₄O(CH₂)₀₋₁-pyridyl which may be substituted with R^{º}; —NO₂; —CN; —N₃; —(CH₂)₀₋₄N(R^{º})₂; —(CH₂)₀₋₄N(R^{º})C(O)R^{º}; —N(R^{º})C(S)R^{º}; —(CH₂)₀₋₄N(R^{º})C(O)NR^{º}₂; —N(R^{º})C(S)NR^{º}₂; —(CH₂)₀₋₄N(R^{º})C(O)OR^{º}; —N(R^{º})N(R^{º})C(O)R^{º}; —N(R^{º})N(R^{º})C(O)NR^{º}₂; —N(R^{º})N(R^{º})C(O)OR^{º}; —(CH₂)₀₋₄C(O)R^{º}; —C(S)R^{º}; —(CH₂)₀₋₄C(O)OR^{º}; —(CH₂)₀₋₄C(O)SR^{º}; —(CH₂)₀₋₄C(O)OSiR^{º}₃; —(CH₂)₀₋₄C(O)R^{º}; —OC(O)(CH₂)₀₋₄SR—, SC(S)SR^{º}; —(CH₂)₀₋₄SC(O)R^{º}; —(CH₂)₀₋₄C(O)NR^{º}2; —C(S)NR^{º}₂; —C(S)SR^{º}; —SC(S)SR^{º}, —(CH₂)₀₋₄C(O)NR^{º}₂; —C(O)N(OR^{º})R^{º}; —C(O)C(O)R^{º}; —C(O)CH₂C(O)R^{º}; —C(NOR^{º})R^{º}; —(CH₂)₀₋₄SSR^{º}; —(CH₂)₀₋₄OS(O)₂R^{º}; —(CH₂)₀₋₄S(O)₂OR^{º}; —(CH₂)₀₋₄OS(O)₂R^{º}; —S(O)₂NR^{º}₂; —(CH₂)₀₋₄S(O)R^{º}; —N(R^{º})S(O)₂NR^{º}₂; —N(R^{º})S(O)₂R^{º}; —N(OR^{º})R^{º}; —C(NH)NR^{º}₂; —P(O)₂R^{º}; —P(O)R^{º}₂; —OP(O)R^{º}₂; —OP(O)(OR^{º})₂; SiR^{º}₃; —(C₁₋₄ straight or branched alkylene)O—N(R^{º})₂; or —(C₁₋₄ straight or branched alkylene)C(O)O—N(R^{º})₂, wherein each R^{º} may be substituted as defined below and is independently hydrogen, C₁-6 aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{º}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{º} (or the ring formed by taking two independent occurrences of R^{º} together with their intervening atoms), are independently halogen, —(CH₂)₀₋₂R^{•}, -(haloR^{•}), —(CH₂)₀₋₂—OH, —(CH₂)₀₋₂—OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂ NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{º} include ═O and ═S.

Suitable divalent substituents on a saturated carbon atom of an “optionally substituted” group include the following: ═O, ═S, ═NNR*₂, ═NNHC(O)R^{•}, ═NNHC(O)OR^{•}, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral center that are not mirror images of one another (diastereomers).

The term “treat” or “treating” means to alleviate symptoms, eliminate the causation of the symptoms either on a temporary or permanent basis, or to inhibit or slow the appearance of symptoms of the named disorder or condition.

The term “therapeutically effective amount” means an amount of the compound which is effective in treating or lessening the severity of one or more symptoms of a disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is pharmaceutically acceptable oil typically used for parenteral administration.

When introducing elements disclosed herein, the articles “a”, “an”, “the”, and “said” are intended to mean that there are one or more of the elements. The terms “comprising”, “having”, “including” are intended to be open-ended and mean that there may be additional elements other than the listed elements.

### III. Compounds

In a first aspect, the present invention provides a compound represented by formula (I):

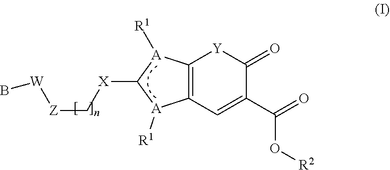

or a pharmaceutically acceptable salt thereof, wherein:
subscript n is 0, 1, or 2;
W is O, NH, or NR″;
X is O or NR″;
Y is O or NR″;
Z is a bond, CH₂, C═O, SO₂;

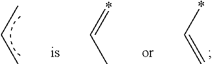

each A is independently selected from the group consisting of N, NR″, S, O, CR″ and CHR″;
each R¹ is independently absent or selected from the group consisting of hydrogen, halogen, C₁₋₆ alkyl, CHF₂, CF₃, CN, —C(O)R″, —C(O)OR″, —SO₂R″, —C(O)NR″₂, —C(O)N(OR″)R″ and —C≡CH;
R² is selected from the group consisting of:
   hydrogen;
   —C(O)R″;
   (CH₂)₀₋₄C(O)R″;
   (CH₂)₀₋₄C(O)OR″;
   optionally substituted C₁₋₆ alkyl;
   an optionally substituted 3-8 membered saturated or partially unsaturated cycloalkyl ring;
   an optionally substituted 3-8 membered saturated or partially unsaturated heterocycloalkyl ring having 1-2 heteroatoms independently selected from nitrogen, oxygen, and sulfur;
   optionally substituted phenyl; and
   an optionally substituted 5-6 membered heteroaryl ring having 1-4 heteroatoms independently selected from nitrogen, oxygen, and sulfur;
B is a ring selected from the group consisting of:
   a 3-8 membered saturated or partially unsaturated monocyclic carbocyclic ring, phenyl,
   a 8-10 membered bicyclic aryl ring,
   a 3-8 membered saturated or partially unsaturated monocyclic or bicyclic heterocyclic ring having 1-2 heteroatoms independently selected from nitrogen, oxygen, and sulfur,
   a 5-6 membered monocyclic heteroaryl ring having 1-4 heteroatoms independently selected from nitrogen, oxygen, and sulfur, and
   a 8-10 membered bicyclic heteroaryl ring having 1-4 heteroatoms independently selected from nitrogen, oxygen, and sulfur,
   wherein B is optionally substituted with one or more substituents selected from R′ and R″;
R′ is selected from the group consisting of OH, C₁₋₆ haloalkyl, C₁₋₆ alkoxy, C₁₋₆ haloalkoxy, and O-phenyl optionally substituted with halogen, C₁₋₆ alkyl, or C₁₋₆ alkoxy;
R″ is selected from the group consisting of: R¹;
   a 3-8 membered saturated or partially unsaturated cycloalkyl ring, optionally substituted with halogen or C₁₋₆ alkyl;
   a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
   phenyl optionally substituted with halogen, C₁₋₆ alkyl, or C₁₋₆ alkoxy; and
   a 5-6 membered heteroaryl ring having 1-4 heteroatoms independently selected from nitrogen, oxygen, and sulfur, said ring optionally substituted with halogen or C₁₋₆ alkyl.

In some embodiments, the present invention provides a compound represented by formula (I):

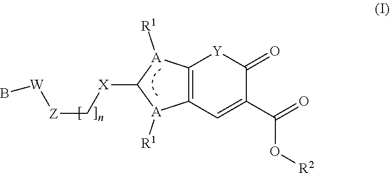

or a pharmaceutically acceptable salt thereof, wherein:
subscript n is 0, 1, or 2;
W is O, NH, or NR″;
X is O or NR″;
Y is O or NR″;
Z is a bond, CH₂, C═O, SO₂;

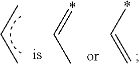

each A is independently selected from the group consisting of N, NR″, S, O, CR″ and CHR″;
R¹, when present, is selected from the group consisting of hydrogen, halogen, C₁₋₆ alkyl, CHF₂, CF₃, CN, —C(O)R″, —C(O)OR″, —SO₂R″, —C(O)NR″₂, —C(O)N(OR″)R″ and —C≡CH;
R² is selected from the group consisting of
   hydrogen;
   —C(O)R″;
   —(CH₂)₀₋₄C(O)R″;
   —(CH₂)₀₋₄C(O)OR″;
   optionally substituted C₁₋₆ alkyl;
   an optionally substituted 3-8 membered saturated or partially unsaturated cycloalkyl ring;
   an optionally substituted 3-8 membered saturated or partially unsaturated heterocycloalkyl ring having 1-2 heteroatoms independently selected from nitrogen, oxygen, and sulfur;
   optionally substituted phenyl; and
   an optionally substituted 5-6 membered heteroaryl ring having 1-4 heteroatoms independently selected from nitrogen, oxygen, and sulfur;
B is a ring selected from the group consisting of:
   a 3-8 membered saturated or partially unsaturated monocyclic carbocyclic ring; phenyl;
   an 8-10 membered bicyclic aryl ring;
   a 3-8 membered saturated or partially unsaturated monocyclic or bicyclic heterocyclic ring having 1-2 heteroatoms independently selected from nitrogen, oxygen, and sulfur;
   a 5-6 membered monocyclic heteroaryl ring having 1-4 heteroatoms independently selected from nitrogen, oxygen, and sulfur; and
   a 8-10 membered bicyclic heteroaryl ring having 1-4 heteroatoms independently selected from nitrogen, oxygen, and sulfur,
   wherein B is optionally substituted with one or more R″ substituents;
R″ is selected from the group consisting of: R¹;
   a 3-8 membered saturated or partially unsaturated cycloalkyl ring, optionally substituted with halogen or C₁₋₆ alkyl;
   a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
   phenyl optionally substituted with halogen or C₁₋₆ alkyl; and
   a 5-6 membered heteroaryl ring having 1-4 heteroatoms independently selected from nitrogen, oxygen, and sulfur, said ring optionally substituted with halogen or C₁₋₆ alkyl.

In some embodiments, when A is ═N—, S, O, NR″, ═CR′—, or CHR″, R¹ attached to the A is absent.

In some embodiments, one A is CR″ and the other A is S, provided each R¹ attached to each A is absent. In some embodiments, one A is CH and the other A is S, provided each R¹ attached to each A is absent.

In some embodiments, the compound of formula (I) is represented by formula (Ia):

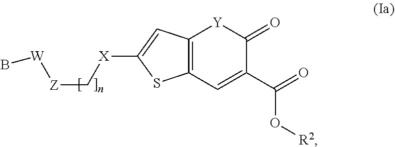

wherein subscript n, B, W, X, Y, Z, and R² are as defined and described herein.

In some embodiments of formula (I) or (Ia), subscript n is 0.

In some embodiments of formula (I) or (Ia), Y is O.

In some embodiments of formula (I) or (Ia), R² is hydrogen.

In some embodiments of formula (I) or (Ia), subscript n is 0; Y is O; and R² is hydrogen.

In some embodiments of formula (I) or (Ia), Z is C═O.

In some embodiments formula (I) or (Ia), subscript n is 0 and Z is C═O. In some embodiments, the compound of formula (I) or (Ia) is represented by formula (Ib):

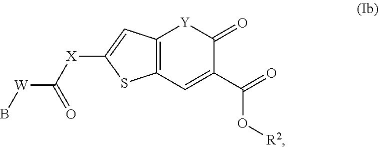

wherein B, W, X, Y, and R² are as defined and described herein.

In some embodiments of any one of formulae (I), (Ia), and (Ib), Y is O.

In some embodiments of any one of formulae (I), (Ia), and (Ib), R² is hydrogen.

In some embodiments, the compound of any one of formulae (I), (Ia), and (Ib) is represented by formula (II):

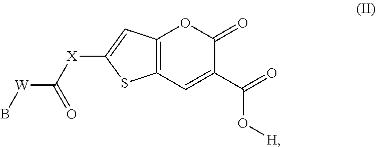

wherein B, W, and X are as defined and described herein.

In some embodiments of formula (I) or (Ia), Z is SO₂. In some embodiments, subscript n is 0 and Z is SO₂. In some embodiments, subscript n is 0, Z is SO₂, and Y is O. In some embodiments, subscript n is 0, Z is SO₂, Y is O, and R² is hydrogen. In some embodiments, the compound of formula (I) or (Ia) is represented by formula (III):

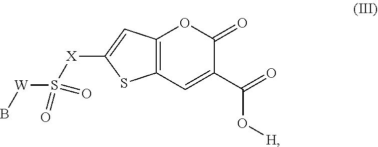

wherein B, W, and X are as defined and described herein.

With reference to any one of formulae (I), (Ia), (Ib), (II) and (III), in some embodiments, X is O. In some embodiments, X is NR″. In some embodiments, R″ is hydrogen. In some embodiments, R″ is C₁₋₆ alkyl. In some embodiments, R″ is methyl. In some embodiments, X is O, NH, or NMe. In some embodiments, X is NH or NMe. In some embodiments, X is NMe.

With reference to any one of formulae (I), (Ia), (Ib), (II) and (III), in some embodiments, W is NH or NR″. In some embodiments, R″ is C₁₋₆ alkyl. In some embodiments, R″ is methyl. In some embodiments, W is NH or NMe. In some embodiments, W is NH. In some embodiments, W is NMe.

In some embodiments, the compound of any one of formulae (I), (Ia), (Ib), and (II) is represented by formula (IIa) or (IIb):

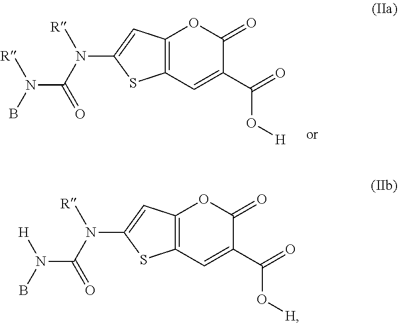

wherein B and each R″ are as defined and described herein.

In some embodiments of formula (IIa), one R″ is hydrogen and the other R″ is C₁₋₆ alkyl. In some embodiments, each R″ is independently C₁₋₆ alkyl. In some embodiments of formula (IIa), one R″ is hydrogen and the other R″ is methyl. In some embodiments, each R″ is methyl.

In some embodiments of formula (IIb), R″ is C₁₋₆ alkyl. In some embodiments of formula (IIb), R″ is methyl.

In some embodiments, the compound of formula (IIa) or (IIb) is selected from the group consisting of:

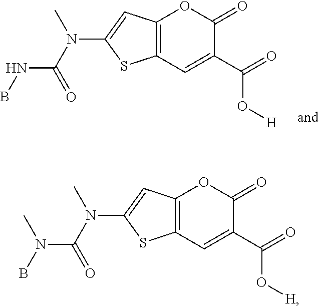

wherein B is as defined and described herein.

With reference to any one of formulae as described herein, in some embodiments, B is a ring selected from the group consisting of:
a 5-6 membered saturated or partially unsaturated monocyclic carbocyclic ring, phenyl,
a 8-10 membered bicyclic aryl ring,
a 5-8 membered saturated or partially unsaturated monocyclic or bicyclic heterocyclic ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
a 8-10 membered bicyclic heteroaryl ring having 1-4 heteroatoms independently selected from nitrogen, oxygen, and sulfur,
wherein B is optionally substituted with one or more substituents selected from R′ and R″.

In some embodiments of B ring as defined and described herein, B is optionally substituted with one or more substituents selected from the group consisting of halogen, OH, CN, C₁₋₆ alkyl, C₁₋₆ haloalkyl, C₃₋₉ cycloalkyl, C₁₋₆ alkoxy, C₁₋₆ haloalkoxy, phenyl, and O-phenyl, wherein each phenyl is optionally independently substituted with halogen, C₁₋₆ alkyl, or C₁₋₆ alkoxy. In some embodiments, B is optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is optionally substituted with one or more substituents selected from the group consisting of F, Cl, OH, CN, methyl, CF₃, OMe, OEt, and OCF₃.

In some embodiments, B ring is phenyl, optionally substituted with one or more substituents selected from R′ and R″, wherein R′ and R″ are as defined and described herein. In some embodiments, B ring is phenyl, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, C₁₋₆ haloalkoxy, and O-phenyl optionally substituted with halogen. In some embodiments, B is phenyl, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is phenyl, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is phenyl, optionally substituted with one or more substituents selected from the group consisting of F, Cl, OH, CN, methyl, CF₃, OMe, OEt, and OCF₃.

In some embodiments, B ring is selected from the group consisting of:

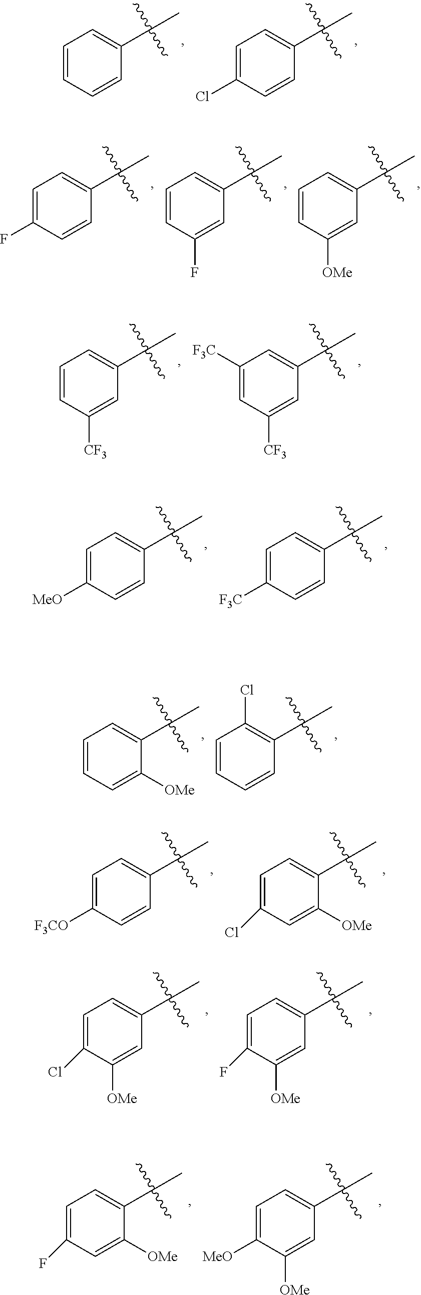

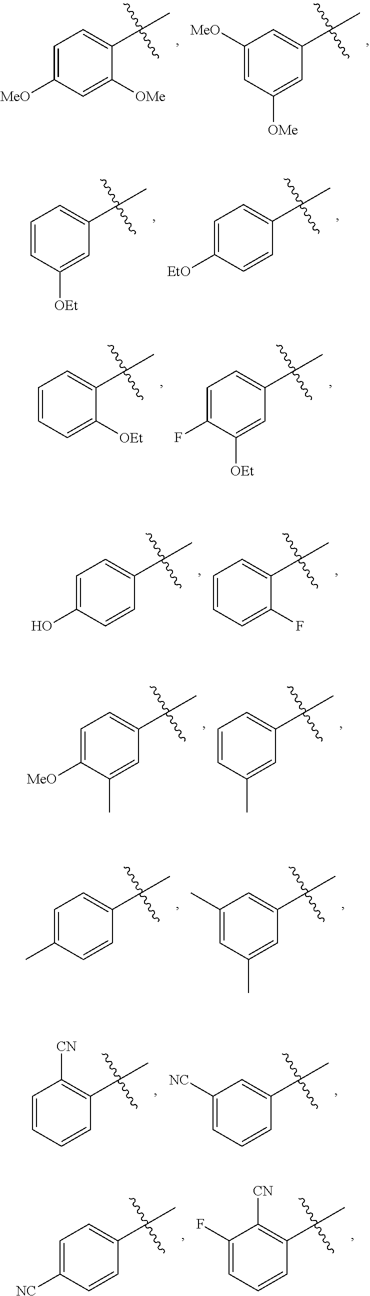

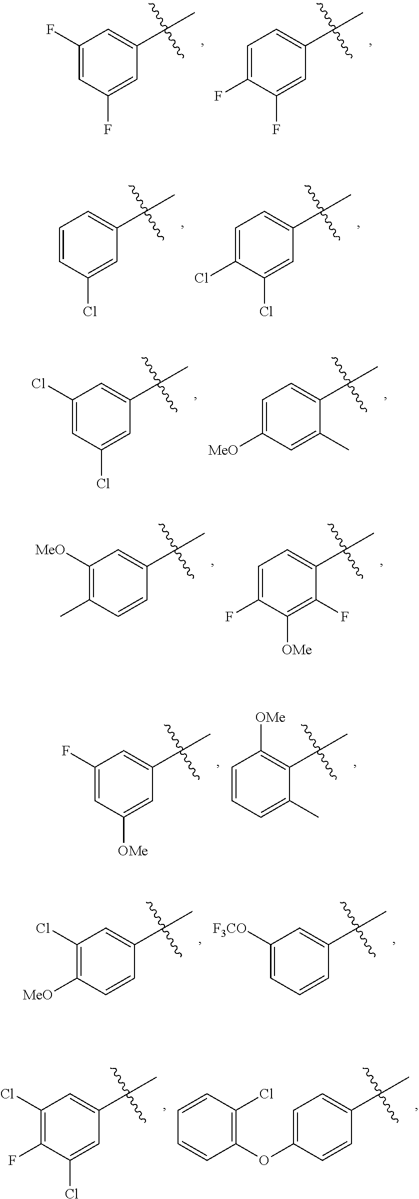

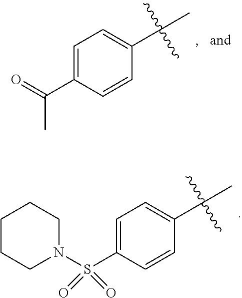

In some embodiments, B ring is a 5-6 membered monocyclic heteroaryl ring having 1-4 heteroatoms independently selected from nitrogen, oxygen, and sulfur, optionally substituted with one or more substituents selected from R′ and R″, wherein R′ and R″ are as defined and described herein. In some embodiments, B ring is a 5-6 membered monocyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₉ cycloalkyl, C₁₋₆ alkoxy, C₁₋₆ haloalkoxy, and phenyl optionally substituted with C₁₋₆ alkoxy. In some embodiments, B is a 5-6 membered monocyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 5-6 membered monocyclic heteroaryl ring having 1-4 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, C(O)Me. In some embodiments, B is a 5-6 membered monocyclic heteroaryl ring having 1-4 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, B ring is selected from the group consisting of:

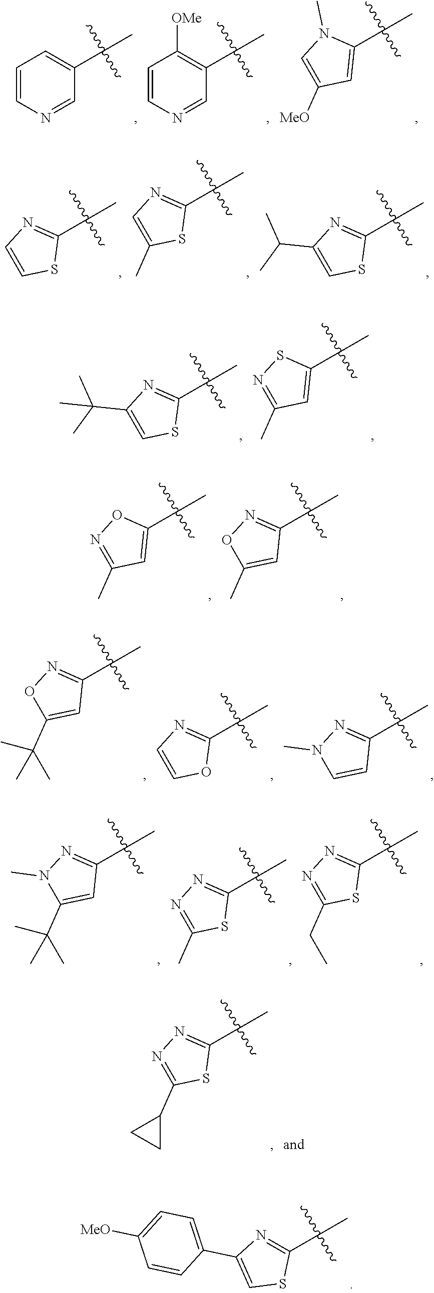

In some embodiments, B ring is a 8-10 membered bicyclic heteroaryl ring having 1-4 heteroatoms independently selected from nitrogen, oxygen, and sulfur, optionally substituted with one or more substituents selected from R′ and R″, wherein R′ and R″ are as defined and described herein. In some embodiments, B ring is a 8-10 membered bicyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₉ cycloalkyl, C₁₋₆ alkoxy, and C₁₋₆ haloalkoxy. In some embodiments, B is a 8-10 membered bicyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 8-10 membered bicyclic heteroaryl ring having 1-4 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is a 8-10 membered bicyclic heteroaryl ring having 1-4 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, B ring is selected from the group consisting of:

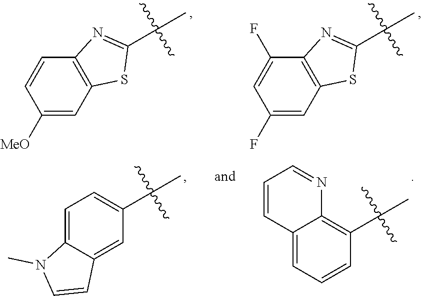

In some embodiments, B ring is cyclopentyl or cyclohexyl, optionally substituted with one or more substituents selected from R′ and R″, wherein R′ and R″ are as defined and described herein. In some embodiments, B ring is cyclopentyl or cyclohexyl, optionally substituted with one or more substituents selected from the group consisting of halogen, OH, CN, C₁₋₆ alkyl, C₁₋₆ haloalkyl, C₃₋₉ cycloalkyl, C₁₋₆ alkoxy, and C₁₋₆ haloalkoxy. In some embodiments, B is cyclopentyl or cyclohexyl, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is cyclopentyl or cyclohexyl, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is cyclopentyl or cyclohexyl, optionally substituted with one or more substituents selected from the group consisting of F, Cl, OH, CN, methyl, CF₃, OMe, OEt, and OCF₃.

In some embodiments, B ring is selected from the group consisting of:

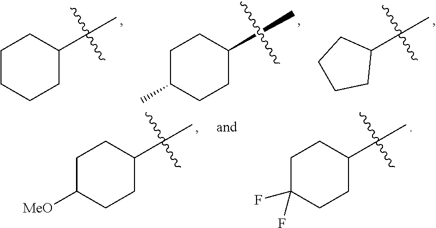

In some embodiments, B ring is a 5-6 membered saturated monocyclic heterocyclic ring having 1-2 heteroatoms independently selected from nitrogen, oxygen, and sulfur, optionally substituted with one or more substituents selected from R′ and R″, wherein R′ and R″ are as defined and described herein. In some embodiments, B ring is a 5-6 membered saturated monocyclic heterocyclic ring having 1-2 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₉ cycloalkyl, C₁₋₆ alkoxy, and C₁₋₆ haloalkoxy. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, B ring is selected from the group consisting of:

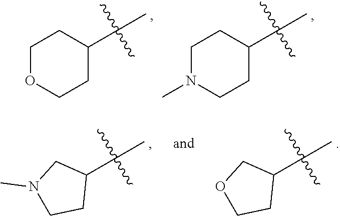

Exemplified compounds of formula (I) or bicyclic enone carboxylic acid compounds are listed in Table 1.

In some embodiments, the present invention provides a compound of formula (I) or a bicyclic enone carboxylic acid compound according to Table 1.

In some embodiments, the compound is selected from the group consisting of:

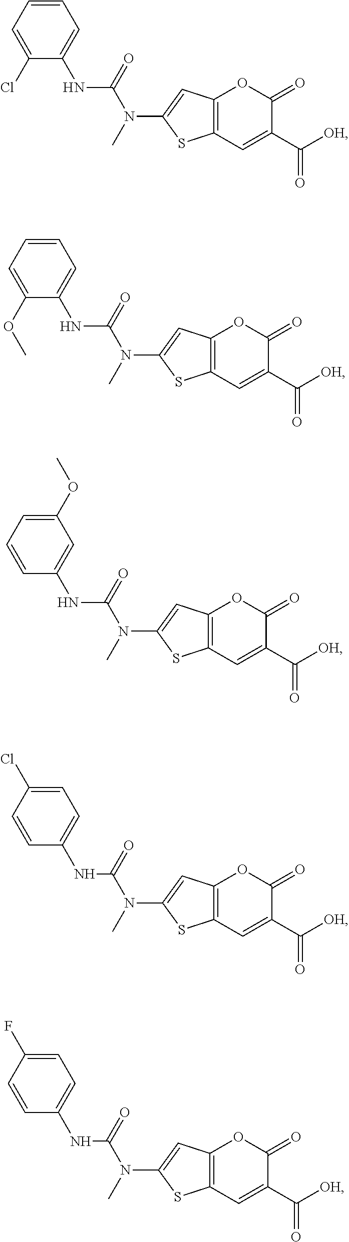 [SMILES: CN(C(=O)Nc1ccccc1Cl)c1cc2oc(=O)c(C(=O)O)cc2s1 ; COc1ccccc1NC(=O)N(C)c1cc2oc(=O)c(C(=O)O)cc2s1 ; COc1cccc(NC(=O)N(C)c2cc3oc(=O)c(C(=O)O)cc3s2)c1 ; CN(C(=O)Nc1ccc(Cl)cc1)c1cc2oc(=O)c(C(=O)O)cc2s1 ; CN(C(=O)Nc1ccc(F)cc1)c1cc2oc(=O)c(C(=O)O)cc2s1]

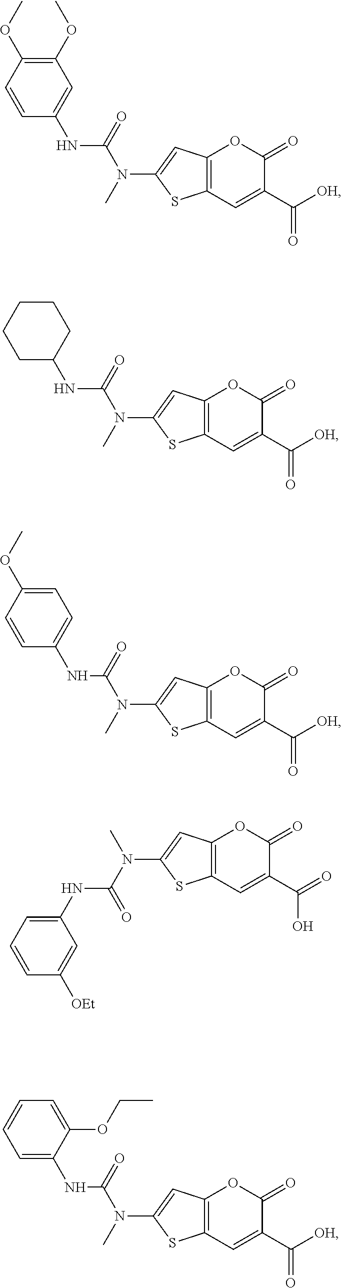 [SMILES: COc1ccc(NC(=O)N(C)c2cc3oc(=O)c(C(=O)O)cc3s2)cc1OC ; CN(C(=O)NC1CCCCC1)c1cc2oc(=O)c(C(=O)O)cc2s1 ; COc1ccc(NC(=O)N(C)c2cc3oc(=O)c(C(=O)O)cc3s2)cc1 ; CCOc1cccc(NC(=O)N(C)c2cc3oc(=O)c(C(=O)O)cc3s2)c1 ; CCOc1ccccc1NC(=O)N(C)c1cc2oc(=O)c(C(=O)O)cc2s1]

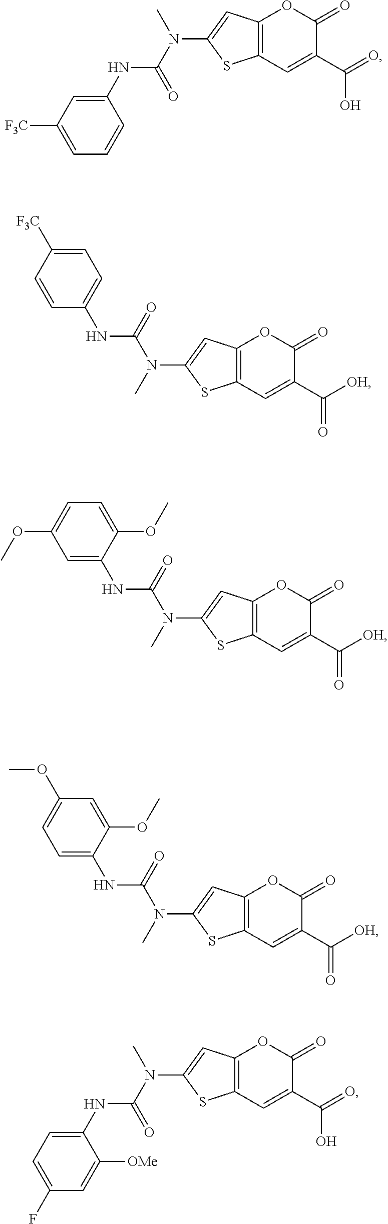 [SMILES: CN(C(=O)Nc1cccc(C(F)(F)F)c1)c1cc2oc(=O)c(C(=O)O)cc2s1 ; CN(C(=O)Nc1ccc(C(F)(F)F)cc1)c1cc2oc(=O)c(C(=O)O)cc2s1 ; COc1ccc(OC)c(NC(=O)N(C)c2cc3oc(=O)c(C(=O)O)cc3s2)c1 ; COc1ccc(NC(=O)N(C)c2cc3oc(=O)c(C(=O)O)cc3s2)c(OC)c1 ; COc1cc(F)ccc1NC(=O)N(C)c1cc2oc(=O)c(C(=O)O)cc2s1]

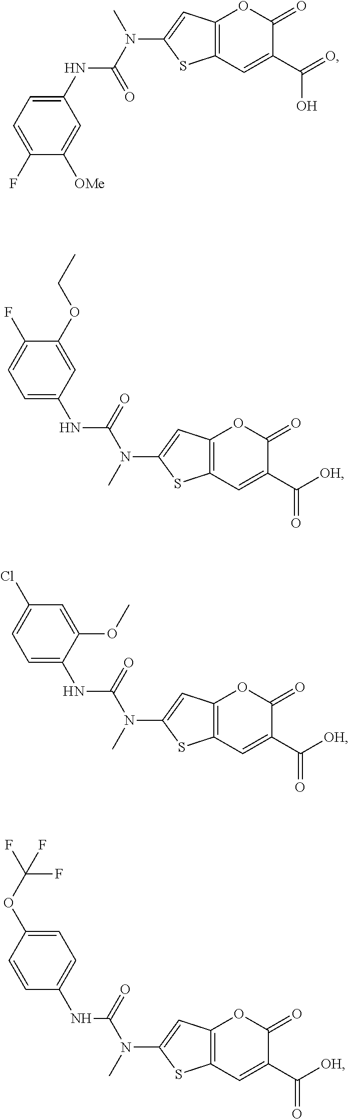 [SMILES: COc1cc(NC(=O)N(C)c2cc3oc(=O)c(C(=O)O)cc3s2)ccc1F ; CCOc1cc(NC(=O)N(C)c2cc3oc(=O)c(C(=O)O)cc3s2)ccc1F ; COc1cc(Cl)ccc1NC(=O)N(C)c1cc2oc(=O)c(C(=O)O)cc2s1 ; CN(C(=O)Nc1ccc(OC(F)(F)F)cc1)c1cc2oc(=O)c(C(=O)O)cc2s1]

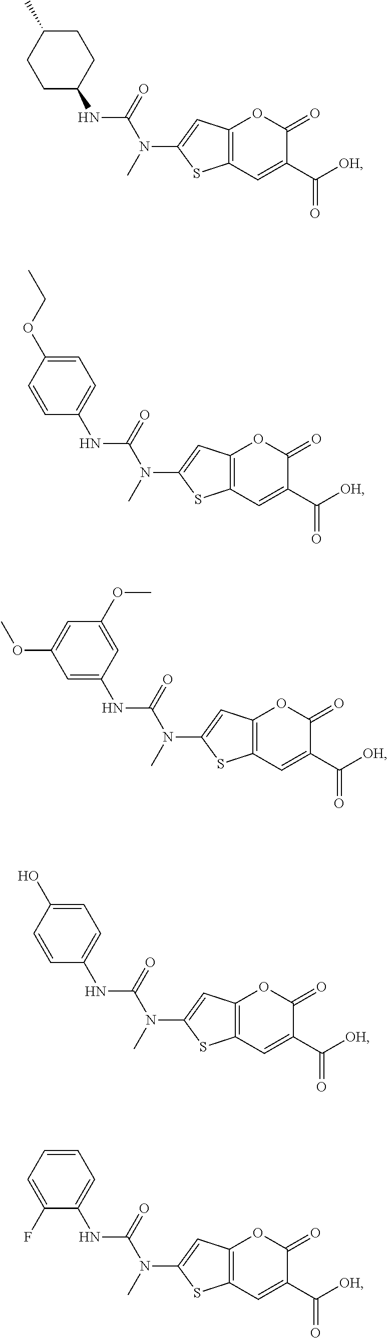 [SMILES: CN(C(=O)N[C@H]1CC[C@H](C)CC1)c1cc2oc(=O)c(C(=O)O)cc2s1 ; CCOc1ccc(NC(=O)N(C)c2cc3oc(=O)c(C(=O)O)cc3s2)cc1 ; COc1cc(NC(=O)N(C)c2cc3oc(=O)c(C(=O)O)cc3s2)cc(OC)c1 ; CN(C(=O)Nc1ccc(O)cc1)c1cc2oc(=O)c(C(=O)O)cc2s1 ; CN(C(=O)Nc1ccccc1F)c1cc2oc(=O)c(C(=O)O)cc2s1]

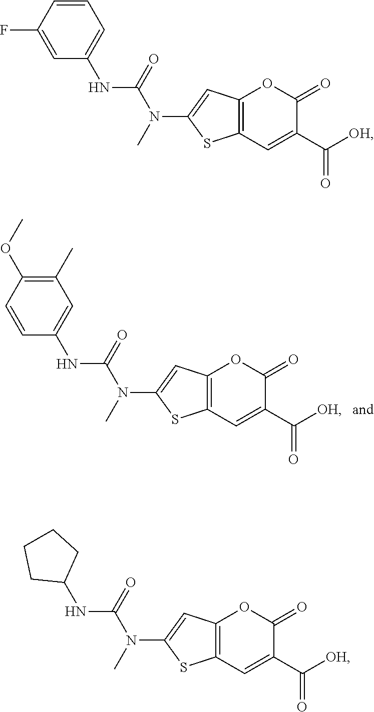 [SMILES: CN(C(=O)Nc1cccc(F)c1)c1cc2oc(=O)c(C(=O)O)cc2s1 ; COc1ccc(NC(=O)N(C)c2cc3oc(=O)c(C(=O)O)cc3s2)cc1C ; "and" ; CN(C(=O)NC1CCCC1)c1cc2oc(=O)c(C(=O)O)cc2s1]

or a pharmaceutically acceptable salt thereof:

In some embodiments, the compound is selected from the group consisting of:

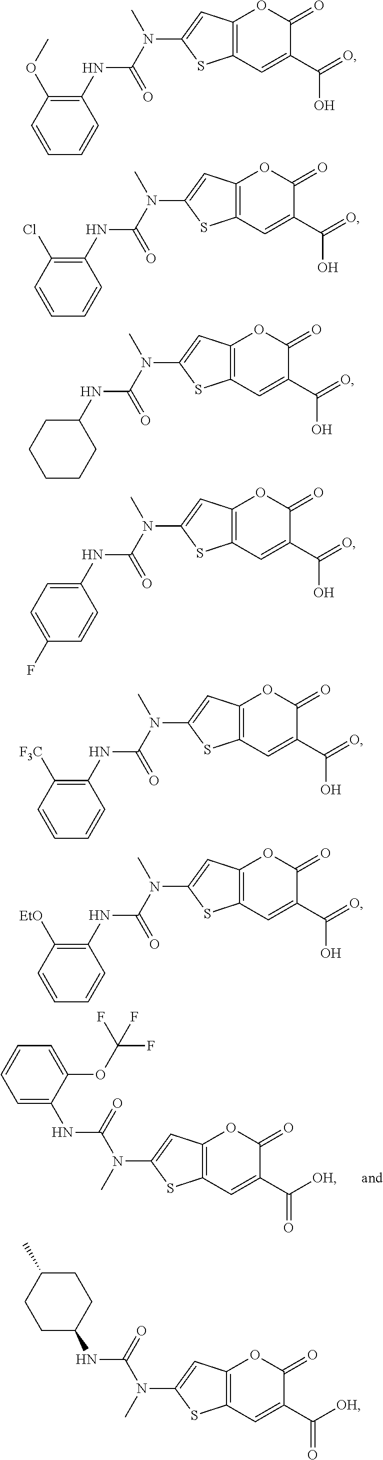

or a pharmaceutically acceptable salt thereof:

In some embodiments, the compound is selected from the group consisting of:

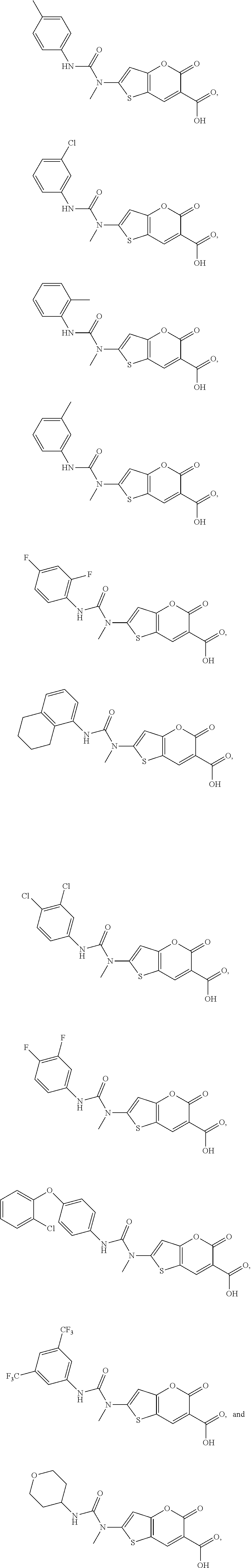

or a pharmaceutically acceptable salt thereof:

In some embodiments, the compound is selected from the group consisting of:

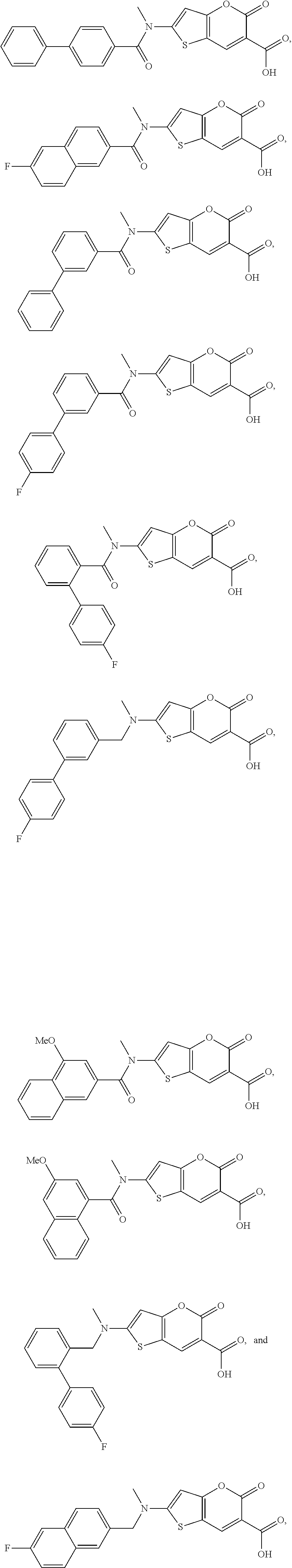

or a pharmaceutically acceptable salt thereof:

In some embodiments, the compound is selected from the group consisting of:

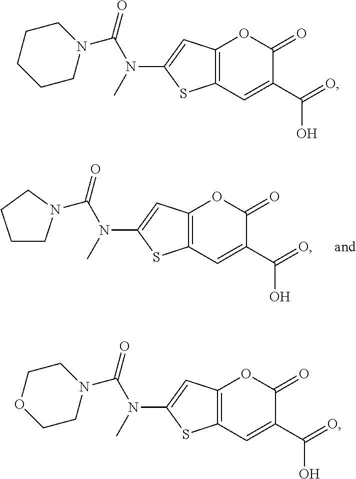 [SMILES: CN(C(=O)N1CCCCC1)c1cc2oc(=O)c(C(=O)O)cc2s1 ; CN(C(=O)N1CCCC1)c1cc2oc(=O)c(C(=O)O)cc2s1 ; CN(C(=O)N1CCOCC1)c1cc2oc(=O)c(C(=O)O)cc2s1]

or a pharmaceutically acceptable salt thereof.

As used herein, and as would be understood by the person of skill in the art, the recitation of “a compound”—unless expressly further limited—is intended to include salts of that compound. Thus, for example, the recitation “a compound of formula (I)” as depicted above, in which R² is H, would include salts in which the carboxylic acid is of the formula COO⁻ M⁺, wherein M is any counterion. In a particular embodiment, the term “compound of formula (I)” refers to the compound or a pharmaceutically acceptable salt thereof. Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the base addition salt is formed from sodium, potassium, magnesium, or calcium.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### IV. Composition

In a second aspect, the present invention provides a pharmaceutical composition including a compound of formula (I) or a compound described herein, or a pharmaceutically acceptable salt thereof, together with a pharmaceutically acceptable carrier.

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J Clin. Pharmacol. 35:1187-1193, 1995; Tjwa, Ann. Allergy Asthma Immunol. 75:107-111, 1995). Accordingly, the present invention also provides pharmaceutical compositions including one or more pharmaceutically acceptable carriers and/or excipients and either a compound of formula (I), or a pharmaceutically acceptable salt of a compound of formula (I).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of Formula (I) mixed with a filler or binders such as lactose or starches, lubricants such as talc or magnesium stearate, and, optionally, stabilizers. In soft capsules, the compound of Formula (I) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of Formula (I) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of Formula (I) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of Formula (I) in a vegetable oil, such as Arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

The pharmaceutical formulations of the compounds of formula (I) of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The pharmaceutical formulations of the compounds of formula (I) of the invention can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

### V. Method

In a third aspect, the present invention provides a method for modulating monocarboxylate transport. The method includes contacting a monocarboxylate transport protein with a therapeutically effective amount of a compound of formula (I), a compound, or a composition thereof described herein.

In a fourth aspect, the present invention provides a method for treating a disorder associated with monocarboxylate transport. The method includes administering a therapeutically effective amount of a compound a compound of formula (I) a compound of formula (I), a compound, or a composition thereof described herein.

In some embodiments, the disorder is selected from the group consisting of cancer, neoplastic disorders, disorders of abnormal tissue growth, disorders of immune system, and tissue and organ rejection.

In some embodiments, the present invention provides a method for treating a neoplastic or metabolic disorder in a subject. The method includes administering a pharmaceutically effective amount of a compound, prodrug thereof, or composition described herein. In some embodiments, the method includes administering a pharmaceutically effective amount of a compound of formula (I), a compound, or a composition thereof described herein.

Also provided herein are methods of treating a disease associated with expression or activity of MCT1, MCT2, MCT3, MCT4, CD147, NFkB, p53 in a subject comprising administering to the patient a therapeutically effective amount of a compound described herein. For example, provided herein are methods of treating various cancers in mammals specifically including humans, dogs, cats, and farm animals, including hematologic malignancies (leukemias, lymphomas, myelomas, myelodysplastic and myeloproliferative syndromes) and solid tumors (carcinomas such as prostate, breast, lung, colon, pancreatic, renal, brain, CNS, skin, cervical, ovarian as well as soft tissue and osteo-sarcomas, and stromal tumors), inflammatory disorders such as rheumatoid arthritis, osteoarthritis, psoriatic arthritis, multiple sclerosis, systemic lupus, systemic sclerosis, vasculitis syndromes (small, medium and large vessel), atherosclerosis, psoriasis and other dermatological inflammatory disorders (such as pemphigus, pemphigoid, allergic dermatitis), and urticarial syndromes comprising administering a compound represented by formula (I).

Also provided are compounds represented by formula I for use in therapy and/or for the manufacture of a medicament for the treatment of a disease associated with expression or activity of MCT1, MCT2, MCT3, MCT4, CD147, NFkB, p53 in a subject.

In some embodiments, the compound or composition is administrable intravenously and/or intraperitoneally and/or orally.

In some embodiments, the invention relates to a composition comprising a compound of this invention or a pharmaceutically acceptable derivative thereof and a pharmaceutically acceptable carrier, adjuvant, or vehicle. The amount of compound in compositions of this invention is such that is effective to measurably inhibit monocarboxylate transport, in a biological sample or in a patient. In certain embodiments, the amount of compound in compositions of this invention is such that is effective to measurably inhibit monocarboxylate transport in a biological sample or in a patient. In certain embodiments, a composition of this invention is formulated for administration to a patient in need of such composition. In some embodiments, a composition of this invention is formulated for oral administration, intravenous, subcutaneous, intraperitoneal or dermatological application to a patient.

The term “patient”, as used herein, means an animal. In some embodiments, the animal is a mammal. In certain embodiments, the patient is a veterinary patient (i.e., a non-human mammal patient). In some embodiments, the patient is a dog. In other embodiments, the patient is a human.

Compounds and compositions described herein are generally useful for the inhibition of monocarboxylate transport. The activity of a compound utilized in this invention as an inhibitor of monocarboxylate transport may be assayed in vitro, in vivo or in a cell line. Detailed conditions for assaying a compound utilized in this invention as an inhibitor of monocarboxylate transport are set forth in the Examples below.

The compounds and compositions described herein can be administered to cells in culture, e.g. in vitro or ex vivo, or to a subject, e.g., in vivo, to treat, prevent, and/or diagnose a variety of disorders.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc, and companion animals (dog, cat, horse etc).

Provided compounds are inhibitors of monocarboxylate transport and are therefore useful for treating one or more disorders associated with activity of monocarboxylate transport. Thus, in certain embodiments, the present invention provides a method for treating a monocarboxylate transport-mediated disorder comprising the step of administering to a patient in need thereof a compound of the present invention, or pharmaceutically acceptable composition thereof:

As used herein, the term “monocarboxylate transport-mediated” disorder or condition, as used herein, means any disease or other deleterious condition in which monocarboxylate transport is known to play a role. Accordingly, another embodiment of the present invention relates to treating or lessening the severity of one or more diseases in which monocarboxylate transport is known to play a role. Specifically, the present invention relates to a method of treating or lessening the severity of a disease or condition selected from a proliferative disorder, wherein said method comprises administering to a patient in need thereof a compound or composition according to the present invention. Such disorders are set forth in detail below.

### Neoplastic Disorders

A compound or composition described herein can be used to treat a neoplastic disorder. A “neoplastic disorder” is a disease or disorder characterized by cells that have the capacity for autonomous growth or replication, e.g., an abnormal state or condition characterized by proliferative cell growth. Exemplary neoplastic disorders include: carcinoma, sarcoma, metastatic disorders (e.g., tumors arising from prostate, colon, lung, breast, cervical, ovarian, liver, melanoma, brain, CNS, head and neck, osteosarcoma, gastrointestinal, pancreatic, hematopoietic neoplastic disorders, e.g., leukemias, lymphomas, myeloma and other malignant plasma cell disorders, and metastatic tumors. Prevalent cancers include: breast, prostate, colon, lung, liver, and pancreatic cancers. Treatment with the compound may be in an amount effective to ameliorate at least one symptom of the neoplastic disorder, e.g., reduced cell proliferation, reduced tumor mass, etc.

The disclosed methods are useful in the prevention and treatment of cancer, including for example, solid tumors, soft tissue tumors, and metastases thereof, as well as in familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the National Cancer Institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

### Cancer Combination Therapies

In some embodiments, a compound described herein is administered together with an additional cancer treatment. Exemplary cancer treatments include, for example: chemotherapy, targeted therapies such as antibody therapies, kinase inhibitors, immunotherapy, immune checkpoint inhibitors, cancer metabolism therapies, hormonal therapy, and anti-angiogenic therapies.

### Immune Activation in the Tumor Microenvironment

In some embodiments, a compound described herein may be used to activate immune cells in the tumor leading to cancer cell killing. Lactate is a metabolite produced from cancer cell metabolism, which suppress the immune system in the local tumor microenvironment. A compound described herein may decrease the lactate content in the tumor microenvironment thus preventing and immune suppression.

### Anti-Angiogenic Therapy

Compounds and methods described herein may be used to prevent or treat a disease or disorder associated with angiogenesis. Diseases associated with angiogenesis include cancer, cardiovascular diseases and macular degeneration. Angiogenesis is the physiological processes involving the growth of new vessels from pre-existing blood vessels. Angiogenesis is the normal and vital process in growth and development, as well as in wound healing and in granular tissue. However, it is also a fundamental step in the transition of tumors from a dormant state to a malignant one. Angiogenesis may be a target for combating diseases characterized by either poor vascularization or abnormal vasculature.

Application of specific compounds that may inhibit the creation of new blood vessels in the body may help combat such diseases. The presence of blood vessels, where there should be none, may affect the normal properties of a tissue, increasing the likelihood of failure. The absence of blood vessels in a repairing or otherwise metabolically active tissue may inhibit repair or other essential functions. Several diseases such as ischemic chronic wounds are the results of failure or insufficient blood vessel formation and may be treated by a local expansion of blood vessels, thus bringing new nutrients to the site, facilitating repair. Other diseases such as age-related macular degeneration may be created by a local expansion of blood vessels, interfering with normal physiological processes.

Vascular endothelial growth factor (VEGF) has been demonstrated to be a major contributor to angiogenesis, increasing the number of capillaries in a given network. Upregulation of VEGF is a major component of the physiological response to exercise and its role in angiogenesis is suspected to be a possible treatment for vascular injuries. In vitro studies clearly demonstrated that VEGF is a potent stimulator of angiogenesis because, in the presence of this growth factor, plated endothelial cells will proliferate and migrate, eventually forming tube structures resembling capillaries.

Tumors induce blood vessel growth by secreting various growth factors (e.g. VEGF). Growth factors such as bFGF and VEGF can induce capillary growth into the tumor, which some researchers suspect supply required nutrients allowing for tumor expansion.

Angiogenesis represents an excellent target for the treatment of cancer and cardiovascular diseases. It is a potent physiological process that underlies the natural manner in which our bodies responds to a diminution of blood supply to vital organs, namely the production of new collateral vessels to overcome the ischemic insult.

Overexpression of VEGF causes increased permeability in blood vessels in addition to stimulating angiogenesis. In wet macular degeneration, VEGF causes proliferation of capillaries into the retina. Since the increase in angiogenesis also causes edema, blood and other retinal fluids leak into the retina causing loss of vision.

Antiangiogenic therapy can include kinase inhibitors targeting vascular endothelial growth factor (VEGF) such as sutinib, sorafenib, monoclonal antibodies, receptor “decoys” to VEGF, VEGF-Trap, thalidomide, its analogs (lenalidomide, pomalidomide), agents targeting non-VEGF angiogenic targets such as fibroblast growth factor (FGF), angiopoietins, angiostatin, or endostatin.

### Immunosuppression

The body's immune system detects foreign objects and organisms such as bacteria, virus, and other pathogens, and protects the body by eliminating those harmful matters. Sometimes, those immune system responses against foreign pathogens or tissues become more harmful to the host, for example, allergies to food and extrinsic antigens such as pollen and respiratory diseases such as asthma. In addition, strong responses against transplant tissues or organs occur leading to the rejection of them. In such cases, immunosuppressive drugs are needed to avoid those complications.

Additionally, the body's immune system does not exert responses against self-tissues or self-antigens under normal circumstances. However, in some cases, body exerts a strong immune response against self-tissues aggressively leading to a variety of autoimmune diseases such as rheumatoid arthritis, multiple sclerosis, type I diabetes, etc. Most immune responses are initiated and controlled by T helper lymphocytes, which respond to antigens.

A number of immunosuppressive therapies have been developed over the last decades. These include rapamycin, which disrupts the cytokine such as IL-2-driven T-cell proliferation by interfering with TOR (Target of Rapamycin) function. However, rapamycin has been shown to cause significant side effects including hyperlipidemia (Hong et al, Semin. Nephrol., 10(2); 108-125, 2000).

### MCTs as Biomarkers and Selection of Patient Sub-Population for the Treatment

Compounds and compositions described herein may also be used to treat selectively sub-population of patients who express either MCT1 or MCT4 or both. It is known that a patient's response to a drug may be dependent upon patient's genetic profile and/or the type of the disease. It has been demonstrated that MCT4 is a biomarker that predicts poor overall survival of aggressive triple negative breast cancer patients.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### VI. EXAMPLES

### Abbreviations

atm Atmosphere
aq. Aqueous
BINAP 2,2′-bis(diphenylphosphino)-1,1′-binaphthyl
Boc tert-butoxycarbonyl
CH₃CN Acetonitrile
CDI N,N′-Carbonyldiimidazole
DCC N,N-Dicyclohexylcarbodiimide
DCM dichloromethane
DBU Diaza(1,3)bicyclo[5.4.0]undecane
DEA Diethylamine
DIEA N,N-Diisopropylethylamine
DIBAL-H Diisobutylaluminium hydride
DIC N,N′-Diisopropylcarbodiimide
DMAP N,N-Dimethyl-4-aminopyridine
DMF Dimethylformamide
DMSO Dimethylsulfoxide
DPPF Diphenylphosphinoferrocene
EA Ethyl acetate
EDCI N-[3-(dimethylamino)propyl]-N′-ethylcarbodiimide hydrochloride
EDC 1-Ethyl-3-(3-dimethylaminopropyl)carbodiimide
Et₂O Diethylether
EtOAc Ethyl acetate
EtOH Ethanol
EtI Iodoethane
Et Ethyl
FCC Flash Column chromatography
Fmoc 9-fluorenylmethyloxycarbonyl
h hour(s)
HetAr Heteroaryl
HOBt N-Hydroxybenzotriazole
HATU 1-[Bis(dimethylamino)methylene]-1H-1,2,3-triazolo[4,5-b]pyridinium 3-oxid hexafluorophosphate
HBTU O-(Benzotriazol-1-yl)-N,N,N′,N′-tetramethyluronium hexafluorophosphate
HPLC High performance liquid chromatography
K₂CO₃ Potassium carbonate
L Leaving group
LAH Lithium aluminium hydride
LCMS HPLC mass spec
MCPBA m-Chlorobenzoic acid
MeCN Acetonitrile
MeOH Methanol
min Minutes
Mel Iodomethane
MeMgCl Methyl magnesium chloride
Me Methyl
n-BuLi 1-Butyllithium
NaOAc Sodium acetate
Na₂SO₄ Sodium sulfate
NMR Monocarboxylate magnetic resonance
NMP N-Methyl pyrrolidinone
nBuLi 1-Butyl lithium
o.n. Over night
RT, rt, r.t. Room temperature
RBF Round-bottomed flask
TEA Triethylamine
THE Tetrahydrofurane
nBu normal Butyl
nM nanomolar
OMs Mesylate or methane sulfonate ester
OTs Tosylate, toluene sulfonate or 4-methylbenzene sulfonate ester
PCC Pyridinium chlorochromate
PPTS Pyridinium p-toluenesulfonate
TBAF Tetrabutylammonium fluoride
TLC Thin Layer Chromatography
TMSI Trimethylsilyliodide
pTsOH p-Toluenesulfonic acid
SPE Solid phase extraction (usually containing silica gel for mini-chromatography)
sat. Saturated
uM micromolar
PG Protecting group
mins minutes

Throughout the following description of such processes it is to be understood that, where appropriate, suitable protecting groups will be added to, and subsequently removed from, the various reactants and Intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any Intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of Intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C. The term “reflux” shall mean, unless otherwise stated, in reference to an employed solvent a temperature at or above the boiling point of named solvent.

### General Synthetic Methods

Several general methods for preparing compounds of Formula I are illustrated in the following Schemes and Examples. Starting materials and the requisite Intermediates are in some cases commercially available or can be prepared according to literature procedures (Bioorg. Med. Chem. 16, 2008, 9487-9497; Med. Chem. Res. 2012; Asian J. Chem. 16, 2004, 1374-1380), or as illustrated herein. In the steps where product was obtained as a mixture of isomers, pure isomers can be easily separated using chromatographic methods in the literature.

It is understood that the functional groups present in compounds described in the Schemes below can be further manipulated, when appropriate, using the standard functional group transformation techniques available to those skilled in the art, to provide desired compounds described in this invention. Other variations or modifications, which will be obvious to those skilled in the art, are within the scope and teachings of this invention.

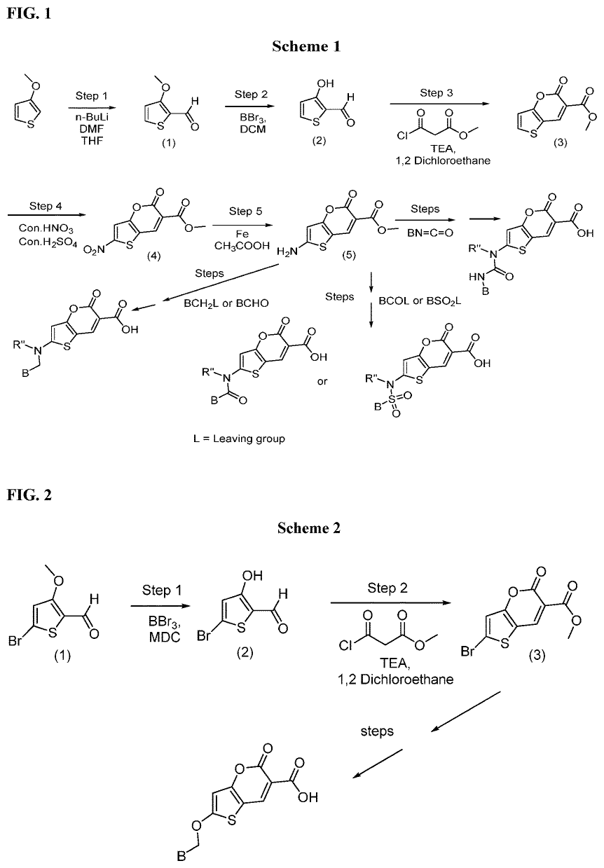

Certain bicyclic enone carboxylic acid compounds of formula (I), wherein the group B is selected from aryl and heteroaryl optionally substituted with one or more substituents and, the X is a nitrogen, n is 0, and the R″ group is an alkyl group can be prepared in accordance with an exemplary Scheme 1 of FIG. 1.

In Scheme 2 of FIG. 2, an exemplary general method is described for the preparation of certain bicyclic enone carboxylic acid compounds.

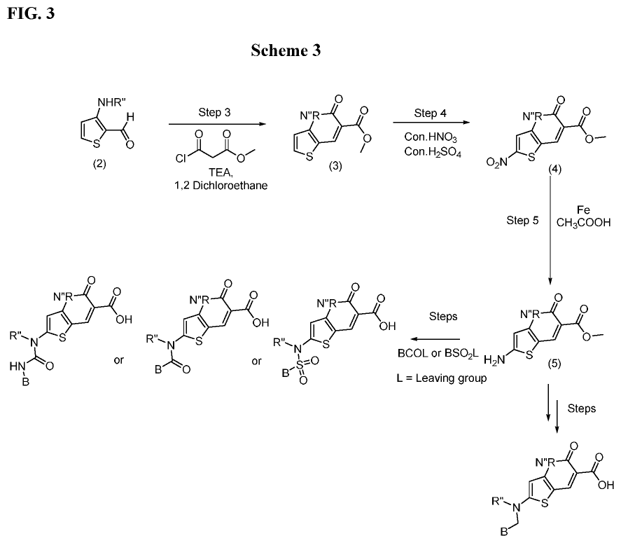

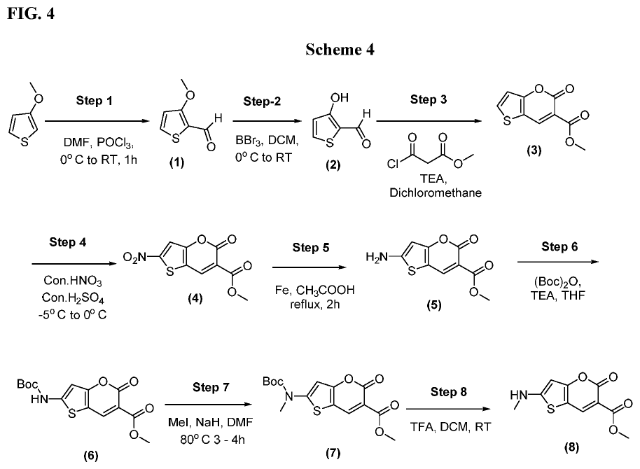

In Scheme 3 of FIG. 3, an exemplary general method is described for the preparation of certain bicyclicenone carboxylic acid compounds of formula (I), wherein the Y is nitrogen providing an amide moiety.

In Scheme 4 of FIG. 4, a method of preparing core structures in formula (I) is also provided. The intermediate (5) was prepared according to Steps 1-5 as detailed below. The intermediate (8) was prepared according to Steps 1-8 as detailed below.

### Step 1

A three-necked RBF was charged with 3-methoxythiophene (100 g, 877.19 mmol, 1.0 eq), and was added N, N-dimethyl formamide (200 mL) followed by dropwise addition of phosphorus (V) oxychloride (98.4 mL, 1052 mmol, 1.2 eq) at 0° C. The reaction mixture was stirred at 0° C. for 1 h. After completion, the reaction mixture was poured into ice-cold water and basified with aqueous sodium hydroxide solution to adjust the pH to ˜9-10. Precipitated solid was filtered and washed with water to obtain 101 g of 3-methoxythiophene-2-carbaldehyde (1); Yield: 81.10%; MS (ES): m/z 143.01 [M+H]⁺; LCMS: 100%; ¹H NMR (400 MHz, DMSO-d6): δ 8.66 (s, 1H), 8.21 (d, J=6, 1H), 8.04 (d, J=9.2, 1H), 4.21-4.27 (m, 4H), 2.10-2.13 (m, 1H), 1.27-1.31 (m, 3H), 0.88-0.90 (m, 6H).

### Step 2

A 3 L, four-necked RBF was charged with boron tribromide (1.0 M in DCM, 1500 mL) followed by dropwise addition of Intermediate (1) (101 g, 711.26 mmol, 1.0 eq) in dichloromethane at 0° C. The reaction mixture stirred at room temperature for 1 h. After completion of reaction, the reaction mixture was transferred into ice-cold water and extracted with dichloromethane. Organic layers were combined, washed with brine solution, dried over anhydrous sodium sulphate and concentrated under reduced pressure to obtain the crude material. The crude was purified by silica gel column chromatography (10% ethyl acetate in hexanes) to obtain 78.0 g of 3-hydroxythiophene-2-carbaldehyde (2); Yield: 85.68%; MS (ES): m/z 129.0 [M+H]; LCMS purity: 100%; ¹H NMR (400 MHz, DMSO-d6): δ 8.66 (s, 1H), 8.21 (d, J=6, 1H), 8.04 (d, J=9.2, 1H), 4.21-4.27 (m, 4H), 2.10-2.13 (m, 1H), 1.27-1.31 (m, 3H), 0.88-0.90 (m, 6H).

### Step 3

A 5 L, four-necked RBF was charged Intermediate (2) (78.0 g, 609.37 mmol, 1.0 eq) in dichloromethane (3900 mL) followed by a dropwise addition of methyl malonyl chloride (77.6 mL, 731.24 mmol, 1.2 eq) at room temperature. The reaction mixture was refluxed for 1.5 h, and then gradually cooled to room temperature. Triethylamine (128 mL, 914.05 mmol, 1.5 eq) was added dropwise to the reaction mixture and stirred at room temperature for 16 h. After completion of reaction, the mixture was concentrated under reduced pressure to obtain the crude material. To this crude product was added ethyl acetate (80 mL) and the mixture was stirred for 20 min to obtain a precipitate which was collected by filtration. The solid was suspended in water and stirred for 10 min followed by second filtration to obtain 42.6 g of methyl 5-oxo-5H-thieno[3,2-b]pyran-6-carboxylate (3); Yield, 33.29%; MS (ES): m/z 211.09 [M+H]; LCMS: 100%; ¹H NMR (400 MHz, DMSO-d6): δ 8.98 (s, 1H), 8.31-8.30 (d, J=5.2, 1H), 7.31-7.30 (d, J=5.2, 1H), 3.80 (s, 3H).

### Step 4

A three-necked RBF was charged with Intermediate (3) (31.0 g, 147.61 mmol, 1.0 eq) in sulfuric acid (186 mL, 6 T) followed by dropwise addition of nitric acid (15.3 mL, 369.02 mmol, 2.5 eq) at 0° C. and the resultant mixture stirred for 30 min. After completion of reaction, the mixture was transferred into ice-cold water and the mixture was extracted with dichloromethane several times. Organic layers were combined, washed with brine solution, dried over anhydrous sodium sulphate, and concentrated under reduced pressure to obtain the crude material. This was purified by silica gel column chromatography (100% dichloromethane) to obtain 16.1 g of methyl 2-nitro-5-oxo-5H-thieno[3,2-b]pyran-6-carboxylate (4); Yield: 53.14%; MS (ES): m/z 255.20 [M+H]; LCMS Purity: 100%; ¹H NMR (400 MHz, DMSO-d6): δ 9.01 (s, 1H), 8.38 (s, 1H), 3.84 (s, 3H).

### Step 5

A three-necked RBF was charged with Intermediate (4) (20.0 g, 78.43 mmol, 1.0 eq) in acetic acid (300 mL) followed by portion wise addition of iron powder (Fe) (30.6 g, 549.01 mmol, 7.0 eq) at room temperature. The reaction mixture stirred 50° C. to 55° C. for 30-40 min. After completion of the reaction, the mixture was filtered and wash with hexanes to obtain a wet cake. The wet cake was further stirred in hexane to remove Fe metal and to obtain 13 g of methyl 2-amino-5-oxo-5H-thieno[3,2-b]pyran-6-carboxylate (5); Yield, 73.65%; MS (ES): m/z 225.22 [M+H]; LCMS purity: 100%; ¹H NMR (400 MHz, DMSO-d6): δ 9.08 (s, 1H), 8.42 (s, 1H), 7.35 (s, 2H), 3.85 (s, 3H).

### Step 6

A three-necked RBF was charged with Intermediate (5) (18 g 80 mmol, 1.0 eq) in DMF (360 mL) followed by addition of 4-dimethylaminopyridine (DMAP) (4.8 g, 40 mmol, 0.5 eq) and di-tert-butyl dicarbonate (20.928 g, 96 mmol, 1.2 eq) at 0° C. Reaction mixture was stirred at room temperature for 5 h. After completion, the reaction mixture was transferred into ice-cold water and the resulting precipitate was collected by filtration and washed with water and hexanes to obtain 14.4 g of methyl 2-((tert-butoxycarbonyl)amino)-5-oxo-5H-thieno[3,2-b]pyran-6-carboxylate (6); Yield, 55.38%); MS (ES): m/z 325.34 [M+H]⁺; LCMS purity: 98.87%; ¹H NMR (400 MHz, DMSO-d6): δ 9.92 (s, 1H), 8.39 (s, 1H), 7.89 (s, 1H), 3.86 (s, 3H), 1.51 (s, 9H).

### Step 7

A three-necked RBF was charged with Intermediate (6) (14.4 g, 44.26 mmol, 1.0 eq) in DMF (290 mL) followed by addition of potassium carbonate (12.2 g, 88.52 mmol, 2.0 eq) and methyl iodide was added dropwise (31.42 g, 221.3 mmol, 5.0 eq) at 0° C. The reaction mixture stirred at 80° C. for 5 h. After completion, the reaction mixture was transferred into ice-cold water and the precipitate was collected by filtration and washed with water and hexanes to obtain 8.4 g of methyl 2-((tert-butoxycarbonyl)(methyl)amino)-5-oxo-5H-thieno[3,2-b]pyran-6-carboxylate (7); Yield, 55.92%; MS (ES): m/z 339.36 [M+H]⁺; LCMS Purity: 99.95%; ¹H NMR (400 MHz, DMSO-d6): δ 8.39 (s, 1H), 7.93 (s, 1H), 3.88 (s, 3H), 3.32 (s, 3H), 1.49 (s, 9H).

### Step 8

A three-necked RBF was charged with Intermediate (7) (8.4 g, 24.75 mmol, 1.0 eq) in dichloromethane (80 mL), and was cooled to 0° C. and trifluoroacetic acid (8 mL) was added. The reaction mixture was stirred at that temperature for 2-3 h. After completion, the reaction mixture was concentrated under reduced pressure to obtain the crude product, which was triturated with ethyl acetate and ether to a obtain 4.2 g of methyl-2-(methylamino)-5-oxo-5H-thieno[3,2-b]pyran-6-carboxylate (8); Yield, 70.92%; MS (ES): m/z 239.25 [M+H]⁺; LCMS: 100%; HPLC: 99.72%; ¹H NMR (DMSO-d6, 400 MHZ): 8.72 (s, 1H), 8.46 (s, 1H), 6.10 (s, 1H), 3.81 (s, 3H), 2.90 (s, 3H).

### Example 1

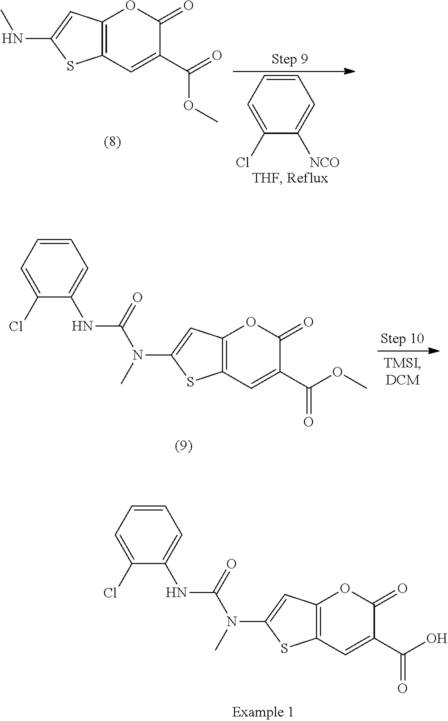

### Step 9

To a three-necked RBF was charged Intermediate (8) from Scheme 4 (0.300 g, 1.25 mmol, 1.0 eq) in tetrahydrofuran (25 mL) was added 1-chloro-2-isocyanatobenzene (0.289 g, 1.88 mmol, 1.5 eq). The reaction mixture was refluxed for 16 h. After completion of reaction, the mixture was transferred into water and extracted several times with dichloromethane. The organic layers were combined, washed with brine, dried over anhydrous sodium sulphate and concentrated under reduced pressure to obtain the crude material. The crude was purified by silica gel column chromatography (2.2% methanol in dichloromethane) to obtain 0.189 g of methyl 2-(3-(2-chlorophenyl)-1-methylureido)-5-oxo-5H-thieno[3,2-b]pyran-6-carboxylate (9); Yield, 38.37%; MS (ES): m/z 392.81 [M+H]⁺; LCMS purity: 97.85%; ¹H NMR (DMSO-d6, 400 MHZ): 8.90 (s, 1H), 8.10-8.08 (d, J=8 Hz, 2H), 7.85 (s, 1H), 7.57-7.7.55 (d, J=8 Hz, 1H), 7.41-7.40 (d, J=4 Hz, 1H), 7.24-7.20 (m, 1H), 3.71 (s, 3H), 3.32 (s, 3H).

### Step 10

To a three-necked RBF charged with Intermediate (9) (0.189 g, 0.482 mmol, 1.0 eq) in dichloromethane (20 mL) and was added trimethylsilyl iodide (0.5 mL, 2.41 mmol, 5.0 eq) at room temperature. The reaction mixture was stirred for 16 h at room temperature. After completion of reaction, the mixture was concentrated under reduced pressure to obtain the crude product which was triturated with methanol to obtain 0.118 g of 2-(3-(2-chlorophenyl)-1-methylureido)-5-oxo-5H-thieno[3,2-b]pyran-6-carboxylic acid (Example 1); Yield, 64.75%; MS (ES): m/z 378.78 [M+H]⁺; LCMS purity: 96.86%; HPLC purity: 95.04%; ¹H NMR (DMSO-d6, 400 MHZ): 12.52 (s, 1H), 9.45 (s, 1H), 8.79 (s, 1H), 7.58-7.48 (m, 2H), 7.41-7.32 (m, 2H), 6.95 (s, 1H), 3.74 (s, 3H).

### Example 2

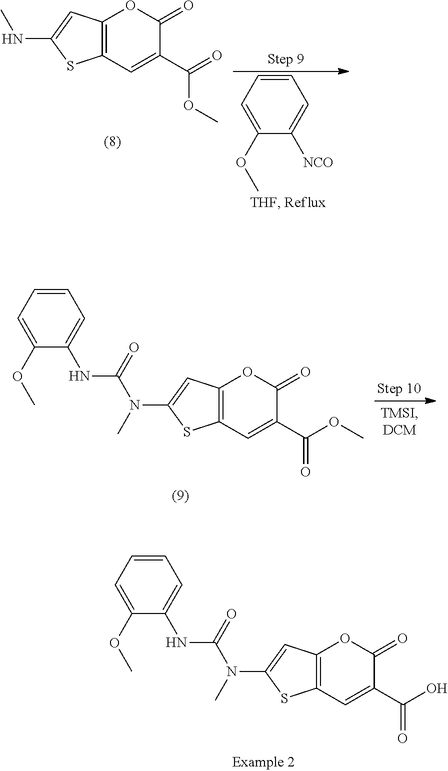

### Step 9

To a three-necked RBF was charged with Intermediate (8) from Scheme 4 (0.300 g, 1.25 mmol, 1.0 eq) in tetrahydrofuran (25 mL) and 1-isocyanato-2-methoxybenzene (0.224 g, 1.50 mmol, 1.2 eq). The reaction mixture was refluxed for 16 h. After completion of reaction, the mixture was transferred into water and extracted with dichloromethane. The organic layers were combined, washed with brine, dried over sodium sulphate and concentrated under reduced pressure to obtain the crude material. The crude was purified by silica gel column chromatography (2.7% methanol in dichloromethane) to obtain 0.150 g of methyl 2-(3-(2-methoxyphenyl)-1-methylureido)-5-oxo-5H-thieno[3,2-b]pyran-6-carboxylate (9); Yield, 30.80%; MS (ES): m/z 388.39 [M+H]⁺; LCMS purity: 97.35%; ¹H NMR (DMSO-d6, 400 MHZ): 8.85 (s, 1H), 8.08 (s, 1H), 7.83-7.81 (d, J=8 Hz, 2H), 7.13-7.07 (m, 3H), 3.80 (s, 3H), 3.68 (s, 3H), 3.30 (s, 3H).

### Step 10

To a three-necked RBF charged with Intermediate (9) (0.150 g, 0.386 mmol, 1.0 eq) in dichloromethane (20 mL) and trimethylsilyl iodide (0.38 mL, 1.93 mmol, 5.0 eq) at room temperature. The reaction mixture was stirred for 16 h at room temperature. After completion of reaction, the mixture was concentrated under reduced pressure to obtain the crude material which was triturated with methanol to obtain 0.101 g of 2-(3-(2-methoxyphenyl)-1-methylureido)-5-oxo-5H-thieno[3,2-b]pyran-6-carboxylic acid (Example 2); Yield: 69.86%; MS (ES): m/z 374.37 [M+H]⁺; LCMS purity: 98.37%; HPLC purity: 96.97%; ¹H NMR (DMSO-d6, 400 MHZ): 12.51 (s, 1H), 8.90 (s, 1H), 8.78 (s, 1H), 7.45-7.43 (d, J=7.2 Hz, 1H), 7.24-7.20 (t, J=7.6 Hz, 1H), 7.11-7.09 (d, J=8 Hz, 1H), 6.98-6.92 (m, 2H), 3.82 (s, 3H), 3.59 (s, 3H).

### Example 3

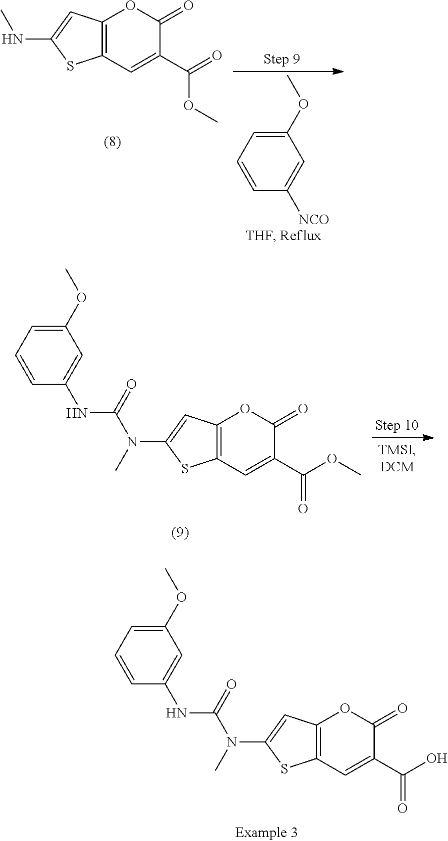

### Step 9

A three-necked RBF charged with Intermediate (8) from Scheme 4 (0.300 g, 1.25 mmol, 1.0 eq) in tetrahydrofuran (25 mL) and 1-isocyanato-3-methoxybenzene (0.224 g, 1.50 mmol, 1.2 eq). The reaction mixture refluxed for 16 h. After completion of reaction, the mixture was transferred into water and extracted with dichloromethane. Organic layers were combined, washed with brine solution, dried over sodium sulphate and concentrated under reduced pressure to obtain the crude material. This was purified by silica gel column chromatography (2.4% methanol in dichloromethane) to obtain 0.190 g of methyl 2-(3-(3-methoxyphenyl)-1-methylureido)-5-oxo-5H-thieno[3,2-b]pyran-6-carboxylate (9); Yield: 39.01%; MS (ES): m/z 388.39 [M+H]⁺; LCMS purity: 97.68%; ¹H NMR (DMSO-d6, 400 MHZ): 8.82 (s, 1H), 8.05 (s, 1H), 7.78 (s, 1H), 7.30-7.25 (m, 3H), 6.62-6.60 (d, J=8.4 Hz, 1H), 3.70 (s, 3H), 3.65 (s, 3H), 3.30 (s, 3H).

### Step 10

A three-necked RBF was charged with Intermediate (9) (0.190 g, 0.489 mmol, 1.0 eq) in dichloromethane (20 mL) and trimethylsilyl iodide (0.35 mL, 2.44 mmol, 5.0 eq) at room temperature. The reaction mixture was stirred for 16 h at room temperature. After completion of reaction, the mixture was concentrated under reduced pressure to obtain the crude material which was triturated with methanol to obtain 0.122 g of 2-(3-(3-methoxyphenyl)-1-methylureido)-5-oxo-5H-thieno[3,2-b]pyran-6-carboxylic acid (Example 3); Yield: 66.62%; MS (ES): m/z 375.25 [M+H]⁺; LCMS purity: 99.31%; HPLC purity: 96.39%; ¹H NMR (DMSO-d6, 400 MHZ): 12.52 (s, 1H), 9.42 (s, 1H), 8.80 (s, 1H), 7.27-7.23 (d, J=8 Hz, 1H), 7.17-7.125 (m, 2H), 6.93 (s, 1H), 6.70-6.68 (d, J=8 Hz, 1H), 3.75 (s, 3H), 3.67 (s, 3H).

### Example 4

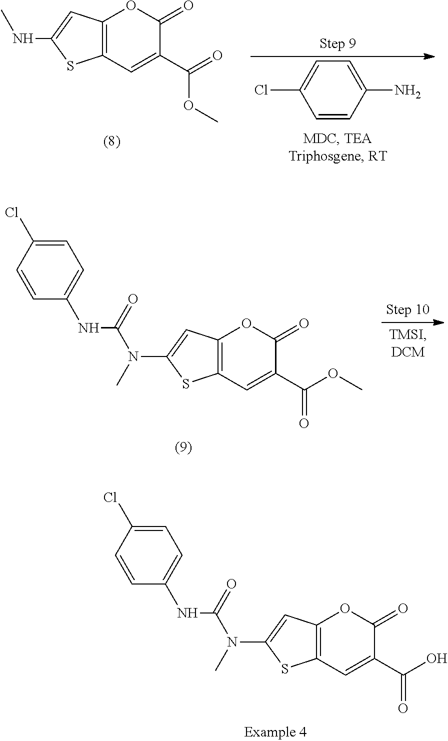 [SMILES: CNc1cc2oc(=O)c(C(=O)OC)cc2s1 ; Nc1ccc(Cl)cc1 ; COC(=O)c1cc2sc(N(C)C(=O)Nc3ccc(Cl)cc3)cc2oc1=O ; CN(C(=O)Nc1ccc(Cl)cc1)c1cc2oc(=O)c(C(=O)O)cc2s1]

### Step 9

A three-necked RBF was charged with 4-chloroaniline (0.5 g, 3.90 mmol, 1.0 eq) in dichloromethane (30 mL) followed by triphosgene (0.273 g, 1.36 mmol, 0.35 eq) at 0° C. After 15 min, Intermediate (8) from Scheme 4 (0.286 g, 1.2 mmol, 0.3 eq) was added followed by triethylamine (1.64 mL, 1.17 mmol, 3.0 eq) dropwise, and the reaction mixture was stirred at room temperature for 3 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.6% methanol in dichloromethane) to obtain 0.140 g of methyl 2-(3-(4-chlorophenyl)-1-methylureido)-5-oxo-5H-thieno[3,2-b]pyran-6-carboxylate (9); Yield: 30.45%; MS (ES): m/z 392.81 [M+H]; LCMS purity: 95.89%; ¹H NMR (DMSO-d6, 400 MHZ): 8.78 (s, 1H), 8.04 (s, 1H), 7.80 (s, 1H), 7.68-7.67 (d, J=6 Hz, 2H), 7.38-7.37 (d, J=6.4 Hz, 2H), 3.65 (s, 3H), 3.25 (s, 3H).

### Step 10

A three-necked RBF was charged with Intermediate (9) (0.140 g, 0.356 mmol, 1.0 eq) in dichloromethane (20 mL), and trimethylsilyl iodide (0.29 mL, 1.786 mmol, 5.0 eq) at room temperature. The reaction mixture was stirred for 16 h at that temperature. After completion of reaction, the mixture was concentrated under reduced pressure to obtain the crude material which was triturated with methanol to obtain 0.085 g of 2-(3-(4-chlorophenyl)-1-methylureido)-5-oxo-5H-thieno[3,2-b]pyran-6-carboxylic acid (Example 4); Yield: 62.96%; MS (ES): m/z 379.05 [M+H]⁺; LCMS purity: 96.24%; HPLC purity: 95.10%; ¹H NMR (DMSO-d6, 400 MHZ): 12.53 (s, 1H), 9.56 (s, 1H), 8.807 (s, 1H), 7.58-7.56 (d, J=8 Hz, 2H), 6.957-6.934 (d, J=8 Hz, 2H), 6.95 (s, 1H), 3.602 (s, 3H).

### Example 5

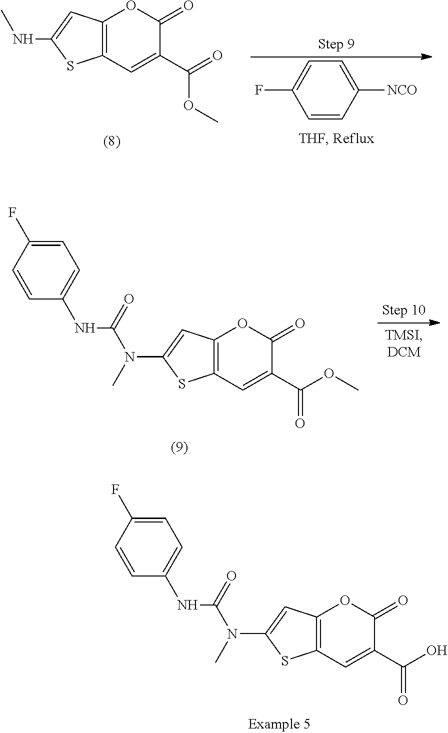

### Step 9

A three-necked RBF was charged with Intermediate (8) from Scheme 4 (0.200 g, 1.25 mmol, 1.0 eq) in tetrahydrofuran (25 mL), and 1-fluoro-4-isocyanatobenzene (0.23 g, 1.69 mmol, 2.0 eq). The reaction mixture was refluxed for 16 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.110 g of methyl 2-(3-(4-fluorophenyl)-1-methylureido)-5-oxo-5H-thieno[3,2-b]pyran-6-carboxylate (9); Yield: 34.96%; MS (ES): m/z 376.36 [M+H]⁺; LCMS purity: 96.91%; ¹H NMR (DMSO-d6, 400 MHZ): 8.70 (s, 1H), 8.04 (s, 1H), 7.78 (s, 1H), 7.10-7.08 (d, J=8 Hz, 2H), 6.84-6.82 (d, J=7.6 Hz, 2H), 3.72 (s, 3H), 3.67 (s, 3H).

### Step 10

A three-necked RBF was charged with Intermediate (9) (0.110 g, 0.292 mmol, 1.0 eq) in dichloromethane (20 mL), and trimethylsilyl iodide (0.3 mL, 1.46 mmol, 5.0 eq) at room temperature. The reaction mixture was stirred for 16 h at that temperature. After completion of reaction, the mixture was concentrated under reduced pressure to obtain the crude material which was triturated with methanol to obtain 0.080 g of 2-(3-(4-fluorophenyl)-1-methylureido)-5-oxo-5H-thieno[3,2-b]pyran-6-carboxylic acid (Example 5); Yield: 75.54%; MS (ES): m/z 362.33 [M+H]⁺; LCMS purity: 95.21%; HPLC purity: 95.95%; ¹H NMR (DMSO-d6, 400 MHZ): 12.52 (s, 1H), 9.49 (s, 1H), 8.79 (s, 1H), 7.55-7.51 (m, 2H), 7.22-7.17 (m, 2H), 6.94 (s, 1H), 3.75 (s, 3H).

### Example 6

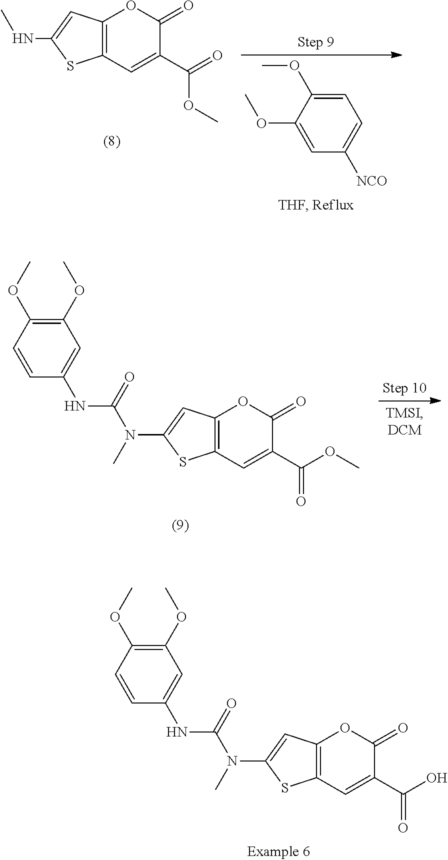

### Step 9

A three-necked RBF was charged with Intermediate (8) from Scheme 4 (0.300 g, 1.25 mmol, 1.0 eq) in tetrahydrofuran (25 mL), and 4-isocyanato-1,2-dimethoxybenzene (0.288 g, 1.80 mmol, 1.5 eq). The reaction mixture was refluxed for 16 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.7% methanol in dichloromethane) to obtain 0.140 g of methyl 2-(3-(3,4-dimethoxyphenyl)-1-methylureido)-5-oxo-5H-thieno[3,2-b]pyran-6-carboxylate (9); Yield: 26.18%; MS (ES): m/z 418.42 [M+H]⁺; LCMS purity: 97.69%; ¹H NMR (DMSO-d6, 400 MHZ): 8.72 (s, 1H), 8.02 (s, 1H), 7.77 (s, 1H), 7.15-7.13 (d, J=8 Hz, 2H), 6.87-6.85 (d, J=6.8 Hz, 2H), 3.81 (s, 3H), 3.68 (s, 3H).

### Step 10

A three-necked RBF was charged with Intermediate (9) (0.140 g, 0.3345 mmol, 1.0 eq) in dichloromethane (20 mL), and trimethylsilyl iodide (0.22 mL, 1.67 mmol, 5.0 eq) at room temperature and reaction mixture was stirred for 16 h at that temperature. After completion of reaction, the mixture was concentrated under reduced pressure to obtain the crude material which was triturated with methanol to obtain 0.098 g of 2-(3-(3,4-dimethoxyphenyl)-1-methylureido)-5-oxo-5H-thieno[3,2-b]pyran-6-carboxylic acid (Example 6); Yield: 72.43%; MS (ES): m/z 404.39 [M+H]⁺; ¹H NMR (DMSO-d6, 400 MHZ): 12.51 (s, 1H), 9.32 (s, 1H), 8.79 (s, 1H), 7.17 (s, 1H), 7.07-7.05 (d, J=8.8 Hz, 1H), 6.94-6.92 (d, J=9.2 Hz, 2H), 3.75 (s, 3H), 3.74 (s, 3H), 3.59 (s, 3H).

### Example 7

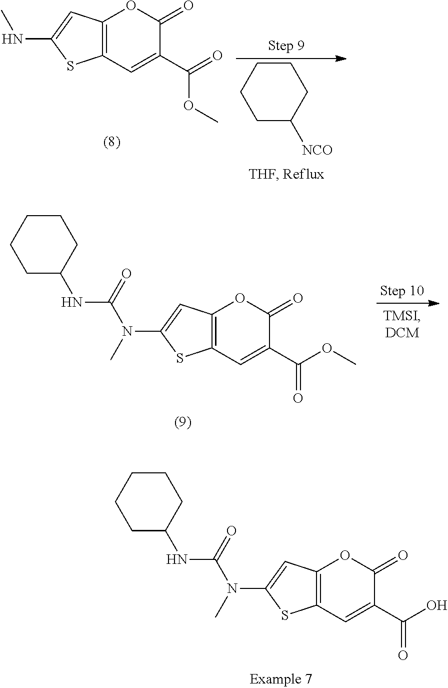

### Step 9

A three-necked RBF was charged with Intermediate (8) from Scheme 4 (0.200 g, 0.8368 mmol, 1.0 eq) in tetrahydrofuran (25 mL), and isocyanatocyclohexane (0.523 g, 4.184 mmol, 5 eq) and the reaction mixture was refluxed for 16 h. After completion of reaction, the mixture was transferred into water and extracted with dichloromethane. Organic layers were combined, washed with brine solution, dried over anhydrous sodium sulphate and concentrated under reduced pressure to obtain the crude material. This was purified by silica gel column chromatography (2.6% methanol in dichloromethane) to obtain 0.120 g of methyl 2-(3-cyclohexyl-1-methylureido)-5-oxo-5H-thieno[3,2-b]pyran-6-carboxylate (9); Yield: 39.39%; MS (ES): m/z 364.42 [M+H]⁺; ¹H NMR (DMSO-d6, 400 MHZ): 8.10 (s, 1H), 7.83 (s, 1H), 6.43 (s, 1H), 3.70 (s, 3H), 3.54-3.53 (m, 1H), 3.29 (s, 3H), 1.74-1.69 (m, 4H), 1.46-1.42 (m, 1H), 1.21-1.11 (m, 4H).

### Step 10

A three-necked RBF was charged with Intermediate (9) (0.189 g, 0.482 mmol, 1.0 eq) in dichloromethane (20 mL), and trimethylsilyl iodide (0.5 mL, 2.41 mmol, 5.0 eq) at room temperature. The reaction mixture was stirred for 16 h at that temperature. After completion of reaction, the mixture was concentrated under reduced pressure to obtain the crude material which was triturated with methanol to obtain 0.118 g of 2-(3-cyclohexyl-1-methylureido)-5-oxo-5H-thieno[3,2-b]pyran-6-carboxylic acid (Example 7); Yield: 78.87%; MS (ES): m/z 350.39 [M+H]⁺; ¹H NMR (DMSO-d6, 400 MHZ): 12.52 (s, 1H), 8.74 (s, 1H), 7.37-7.35 (d, j=7.2 Hz 1H), 6.78 (s, 1H), 3.40 (s, 1H), 1.80-1.73 (q, 2H), 1.61-1.58 (q, 3H), 1.51-1.49 (m, 2H), 1.29-1.22 (m, 4H), 1.11-1.02 (m, 2H).

### Example 8

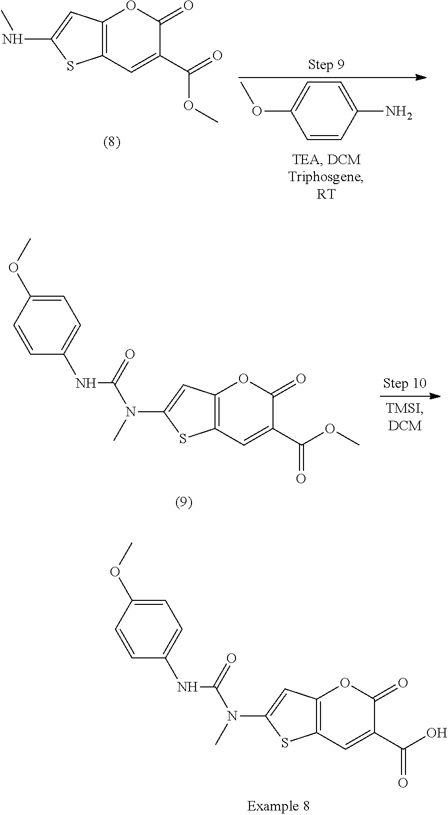

### Step 9

A three-necked RBF was charged 4-methoxyaniline (0.5 g, 4.065 mmol, 1.0 eq) in dichloromethane (30 mL) and triphosgene (0.42 g, 1.42 mmol, 0.3 eq) at 0° C. After stirring 15 min, Intermediate (8) from Scheme 4 (0.19 g, 0.813 mmol, 0.2 eq) was added followed by triethylamine (1.6 mL, 12.19 mmol, 3.0 eq). The reaction mixture stirred at room temperature for 2 h. After completion of reaction, the mixture was transferred into water and extracted with dichloromethane. Organic layers were combined, washed with brine, dried over anhydrous sodium sulphate and concentrated under reduced pressure to obtain the crude material. The crude product was purified by silica gel column chromatography (2.2% methanol in dichloromethane) to obtain 0.120 g of methyl 2-(3-(4-methoxyphenyl)-1-methylureido)-5-oxo-5H-thieno[3,2-b]pyran-6-carboxylate (9); Yield: 23.36%; MS (ES): m/z 388.39 [M+H]⁺; ¹H NMR (DMSO-d6, 400 MHZ): 8.78 (s, 1H), 8.65 (s, 1H), 7.69 (s, 1H), 7.22-7.20 (d, J=8 Hz, 2H), 6.91-6.89 (d, J=7.6 Hz, 2H), 3.71 (s, 3H), 3.62 (s, 3H), 3.25 (s, 3H).

### Step 10

A three-necked RBF was charged with Intermediate (9) (0.120 g, 0.309 mmol, 1.0 eq) in dichloromethane (20 mL) and trimethylsilyl iodide (0.3 mL, 1.546 mmol, 5.0 eq) at room temperature. The reaction mixture was stirred for 16 h at that temperature. After completion of reaction, the mixture was concentrated under reduced pressure to obtain the crude material which was triturated with methanol to obtain 0.105 g of 2-(3-(4-methoxyphenyl)-1-methylureido)-5-oxo-5H-thieno[3,2-b]pyran-6-carboxylic acid (Example 8); Yield: 77.90%; MS (ES): m/z 363.00 [M+H]⁺; LCMS purity: 95.04%; ¹H NMR (DMSO-d6, 400 MHZ): 12.47 (s, 1H), 9.35 (s, 1H), 8.80 (s, 1H), 7.43-7.41 (d, J=8.8 Hz, 2H), 6.95-6.93 (d, J=9.2 Hz, 3H), 3.76 (s, 3H), 3.60 (s, 3H).

### Example 9

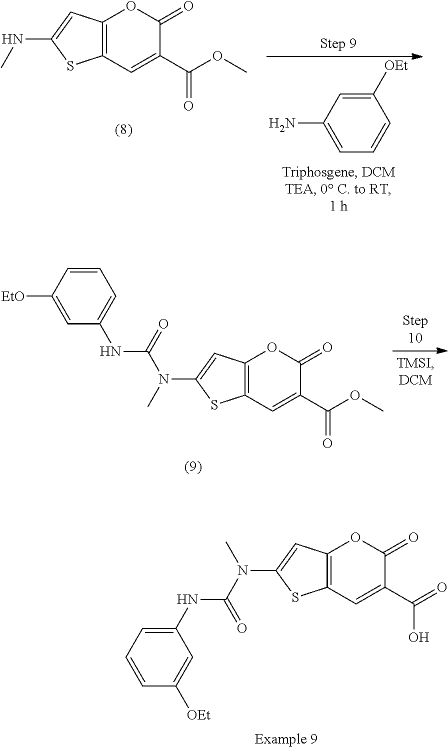

### Step 9

A three-necked RBF was charged with a solution of 3-ethoxyaniline (0.5 g, 3.65 mmol, 1.0 eq) in dichloromethane (30 mL), and triphosgene (0.379 g, 1.28 mmol, 0.35 eq) at 0° C. After 15 min, Intermediate (8) from Scheme 4 (0.261 g, 1.1 mmol, 0.3 eq) was added followed by addition of triethylamine (1.5 mL, 10.93 mmol, 3.0 eq) dropwise into the reaction mixture, and the mixture was stirred at room temperature for 3 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.6% methanol in dichloromethane) to obtain 0.130 g of methyl 2-(3-(3-ethoxyphenyl)-1-methylureido)-5-oxo-5H-thieno[3,2-b]pyran-6-carboxylate (9); Yield: 17.04%); MS (ES): m/z 403.42 [M+H]⁺; ¹H NMR (DMSO-d6, 400 MHZ): 8.70 (s, 1H), 8.62 (s, 1H), 7.77 (s, 1H), 7.30-7.20 (m, 3H), 6.66-6.64 (d, J=8 Hz, 1H), 4.07-4.03 (m, 2H), 3.62 (s, 3H), 3.22 (s, 3H), 1.36-1.30 (m, 3H).

### Step 10

A three-necked RBF was charged with Intermediate (9) (0.130 g, 0.323 mmol, 1.0 eq) in dichloromethane (20 mL) and trimethylsilyl iodide (0.29 mL, 1.6165 mmol, 5.0 eq) at room temperature. The reaction mixture was stirred for 16 h at that temperature. After completion of reaction, the mixture was concentrated under reduced pressure to obtain the crude material which was triturated with methanol to obtain 0.07 g of 2-(3-(3-ethoxyphenyl)-1-methylureido)-5-oxo-5H-thieno[3,2-b]pyran-6-carboxylic acid (Example 9); Yield: 55.79%; MS (ES): m/z 389.39 [M+H]⁺; ¹H NMR (DMSO-d6, 400 MHZ): 12.55 (s, 1H), 9.406 (s, 1H), 8.810 (s, 1H), 7.26-7.24 (t, J=8 Hz, 1H), 7.17 (s, 1H), 7.12-7.10 (d, J=8 Hz, 1H), 6.94 (s, 1H), 6.69-6.67 (d, J=8 Hz, 1H), 4.04-4.02 (q, J=8 Hz, 2H), 3.602 (s, 3H), 1.34-1.32 (d, J=8 Hz, 3H).

### Example 10

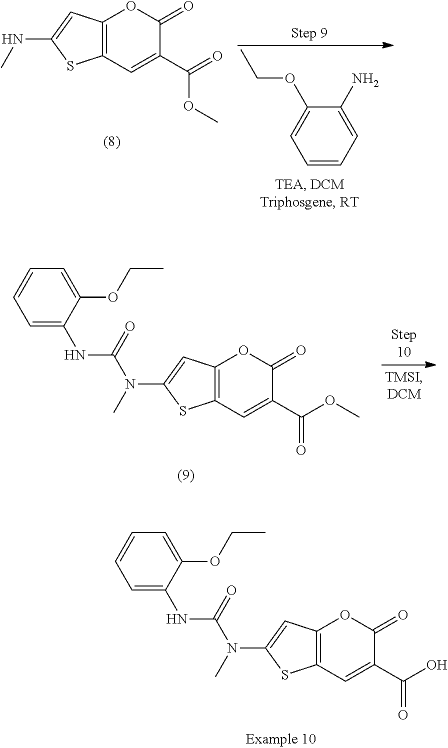

### Step 9

A three-necked RBF was charged with a solution of 2-ethoxyaniline (0.17 g, 0.6276 mmol, 1.5 eq) in dichloromethane (30 mL) and triphosgene (0.065 g, 0.2197 mmol, 0.35 eq) at 0° C. After 15 min, Intermediate (8) from Scheme 4 (0.2 g, 0.4184 mmol, 1.0 eq) was added followed by triethylamine (0.253 g, 1.2552 mmol, 3.0 eq) dropwise into reaction mixture and stirred at room temperature for 2 h. After completion of reaction, the mixture was transferred into water and extracted with dichloromethane. Organic layers were combined, washed with brine solution, dried over anhydrous sodium sulphate and concentrated under reduced pressure to obtain crude material. This was purified by column chromatography (2.2% methanol in dichloromethane) to obtain 0.112 g of methyl 2-(3-(2-ethoxyphenyl)-1-methylureido)-5-oxo-5H-thieno[3,2-b]pyran-6-carboxylate (9); Yield: 33.29%); MS (ES): m/z 402.42 [M+H]⁺; ¹H NMR (DMSO-d6, 400 MHZ): 8.85 (s, 1H), 8.15 (s, 1H), 7.80 (s, 1H), 7.101-7.08 (m, 4H), 4.97-4.96 (m, 2H), 3.71 (s, 3H), 3.32 (s, 3H), 1.34-1.32 (t, J=8 MHz, 3H).

### Step 10

A three-necked RBF was charged with Intermediate (9) (0.140 g, 0.3482 mmol, 1.0 eq) in dichloromethane (20 mL) and trimethylsilyl iodide (0.22 mL, 1.67 mmol, 5.0 eq) at room temperature. The reaction mixture was stirred for 16 h at that temperature. After completion of reaction the mixture was concentrated under reduced pressure to obtain the crude material which was triturated with methanol to obtain 0.112 g of 2-(3-(2-ethoxyphenyl)-1-methylureido)-5-oxo-5H-thieno[3,2-b]pyran-6-carboxylic acid (Example 10); Yield: 82.89%); MS (ES): m/z 388.39 [M+H]⁺; ¹H NMR (DMSO-d6, 400 MHZ): 12.506 (s, 1H), 8.7969 (s, 2H), 7.521-7.502 (d, J=7.6 MHz 1H), 7.210-7.171 (t, J=7.6 MHz, 1H), 7.101-7.081 (d, J=8 MHz, 1H), 6.979-6.944 (t, J=7.6 MHz, 2H), 4.116-4.065 (m, 2H), 3.612 (s, 3H), 1.354-1.319 (t, J=6.8 MHz, 3H).

### Example 11

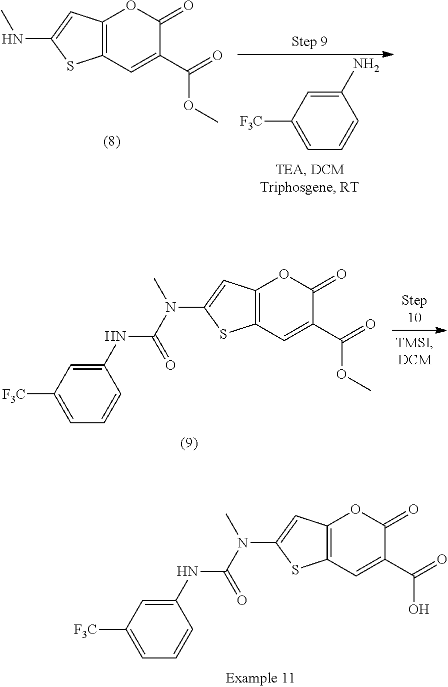

### Step 9

A three-necked RBF was charged with a solution of 3-(trifluoromethyl) aniline (0.222 g, 1.3807 mmol, 1.5 eq) in dichloromethane (30 mL). Triphosgene (0.150 g, 1.378 mmol, 0.35 eq) at 0° C. After 15 min, Intermediate (8) from Scheme 4 (0.220 g, 0.9205 mmol, 1.0 eq) was added followed by triethylamine (0.6 mL, 4.81 mmol, 5.0 eq) dropwise into the reaction mixture and stirred at room temperature for 2 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.132 g of methyl 2-(1-methyl-3-(3-(trifluoromethyl)phenyl)ureido)-5-oxo-5H-thieno[3,2-b]pyran-6-carboxylate (9); Yield: 83.52%; MS (ES): 427.27 m/z [M+H]⁺; ¹H NMR (DMSO-d6, 400 MHZ): 8.90 (s, 1H), 8.09 (s, 1H), 7.97 (s, 1H), 7.85 (m, 1H), 7.56-7.49 (m, 2H), 7.40 (s, 1H), 3.69 (s, 3H), 3.29 (s, 3H).

### Step 10

A three-necked RBF was charged with Intermediate (9) (0.130 g, 0.305 mmol, 1.0 eq) in dichloromethane (20 mL), and trimethylsilyl iodide (0.3 mL, 1.5255 mmol, 5.0 eq) at room temperature. The reaction mixture was stirred for 16 h at that temperature. After completion of reaction, the mixture was concentrated under reduced pressure to obtain the crude material which was triturated with methanol to obtain 0.105 g of 2-(1-methyl-3-(3-(trifluoromethyl)phenyl)ureido)-5-oxo-5H-thieno[3,2-b]pyran-6-carboxylic acid (Example 11); Yield: 83.36%; MS (ES): m/z 413.34 [M+H]⁺; ¹H NMR (DMSO-d6, 400 MHZ): 12.56 (s, 1H), 9.78 (s, 1H), 8.83 (s, 1H), 7.80-7.72 (m, 4H), 6.99 (s, 1H), 3.64 (s, 3H).

### Example 12

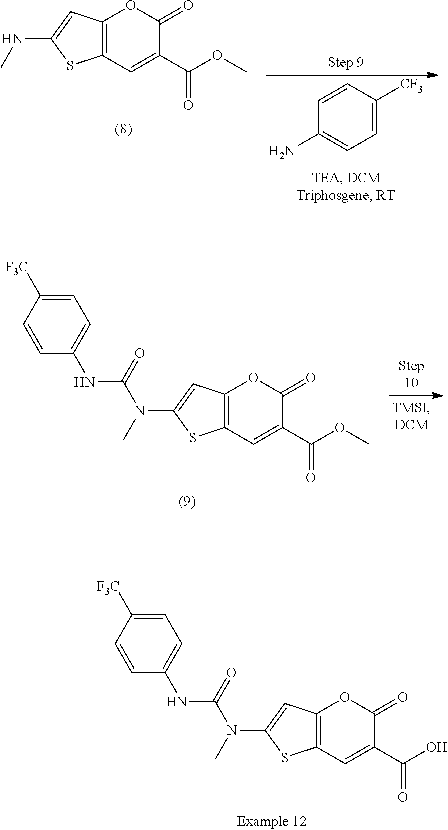

### Step 9

A three-necked RBF was charged with a solution of 4-(trifluoromethyl)aniline (0.250 g, 1.5687 mmol, 1.50 eq) in dichloromethane (30 mL). Triphosgene was added (0.160 g, 0.549 mmol, 0.35 eq) at 0° C. After 15 min Intermediate (8) from Scheme 4 (0.250 g, 1.0458 mmol, 1.0 eq) was added followed by triethylamine (0.7 mL, 5.229 mmol, 5.0 eq) dropwise into reaction mixture and the mixture was stirred at room temperature for 2 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.140 g of methyl 2-(1-methyl-3-(4-(trifluoromethyl)phenyl)ureido)-5-oxo-5H-thieno[3,2-b]pyran-6-carboxylate (9); Yield: 31.42%; MS (ES): m/z 427.37 [M+H]⁺; LCMS purity: 97.55%; ¹H NMR (DMSO-d6, 400 MHZ): 8.73 (s, 1H), 8.02 (s, 1H), 7.78 (s, 1H), 7.552-7.532 (d, J=8 Hz, 2H), 7.431-7.418 (d, J=5.2 Hz, 2H), 3.65 (s, 3H), 3.27 (s, 3H).

### Step 10

A three-necked RBF was charged with Intermediate (9) (0.140 g, 0.3283 mmol, 1.0 eq) in dichloromethane (20 mL). Trimethylsilyl iodide (0.23 mL, 1.6415 mmol, 5.0 eq) was added at room temperature and the reaction mixture was stirred for 16 h at that temperature. After completion of reaction, the mixture was concentrated under reduced pressure to obtain the crude material which was triturated with methanol to obtain 0.102 g of 2-(1-methyl-3-(4-(trifluoromethyl)phenyl)ureido)-5-oxo-5H-thieno[3,2-b]pyran-6-carboxylic acid (Example 12); Yield: 75.34%; MS (ES): m/z 413.34 [M+H]⁺; ¹H NMR (DMSO-d6, 400 MHZ): 12.562 (s, 1H), 9.771 (s, 1H), 8.8.17 (s, 1H), 7.792-7.712 (m, 4H), 6.982 (s, 1H), 3.631 (s, 3H).

### Example 13

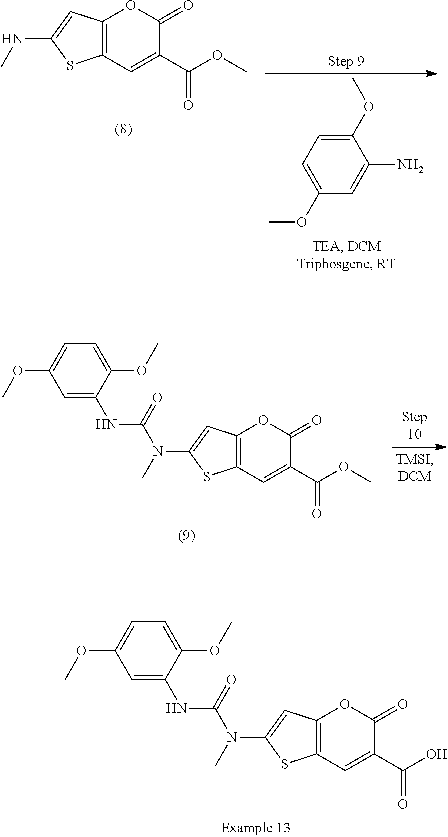

### Step 9

A three-necked RBF was charged with a solution of 2,5-dimethoxyaniline (0.220 g, 1.44 mmol, 1.50 eq) in dichloromethane (30 mL). Triphosgene (0.150 g, 0.505 mmol, 0.35 eq) was added at 0° C. After 15 min Intermediate (8) from Scheme 4 (0.23 g, 0.962 mmol, 1.0 eq) was added followed by triethylamine (0.6 mL, 4.81 mmol, 5.0 eq) dropwise into reaction mixture. The mixture was stirred at room temperature for 2 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.140 g of methyl 2-(3-(2,5-dimethoxyphenyl)-1-methylureido)-5-oxo-5H-thieno[3,2-b]pyran-6-carboxylate (9); Yield: 34.80%; MS (ES): m/z 418.42 [M+H]⁺; ¹H NMR (DMSO-d6, 400 MHZ): 8.90 (s, 1H), 8.10 (s, 1H), 7.85 (s, 1H), 7.51-7.50 (d, J=8.8 Hz, 1H), 7.03-7.01 (d, J=8 Hz 1H), 6.791-6.761 (m, 1H), 3.85 (s, 3H), 3.74 (s, 3H), 3.71 (S, 3H), 3.32 (s, 3H).

### Step 10

A three-necked RBF was charged with Intermediate (9) (0.140 g, 0.334 mmol, 1.0 eq) in dichloromethane (20 mL), and trimethylsilyl iodide (0.22 mL, 1.67 mmol, 5.0 eq) at room temperature. The reaction mixture was stirred for 16 h at that temperature. After completion of reaction the mixture was concentrated under reduced pressure to obtain the crude material which was triturated with methanol to obtain 0.107 g of 2-(3-(2,5-dimethoxyphenyl)-1-methylureido)-5-oxo-5H-thieno[3,2-b]pyran-6-carboxylic acid (Example 13); Yield: 79.08%; MS (ES): m/z 404.39 [M+H]⁺; ¹H NMR (DMSO-d6, 400 MHZ): 12.516 (s, 1H), 8.860-8.777 (m, 2H), 7.165-7.158 (d, J=2.8 Hz 1H), 7.036-7.014 (d, J=8.8 Hz, 1H), 6.939 (s, 1H), 6.791-6.761 (m, 1H), 3.825 (s, 3H), 3.775 (s, 3H), 3.600 (S, 3H).

### Example 14

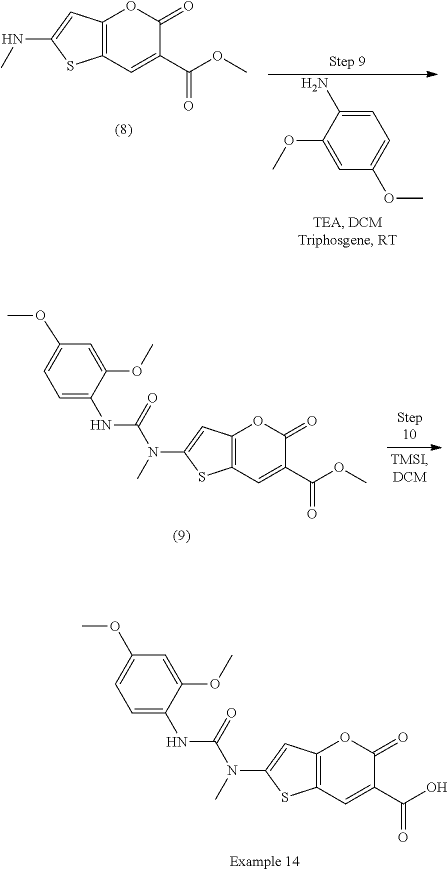

### Step 9

A three-necked RBF was charged with a solution of 2,4-dimethoxyaniline (0.192 g, 1.25 mmol, 1.5 eq) in dichloromethane (30 mL). Triphosgene (0.130 g, 0.439 mmol, 0.35 eq) was added at 0° C. After 15 min, Intermediate (8) from Scheme 4 (0.200 g, 0.836 mmol, 1.0 eq) was added followed by triethylamine (0.6 mL, 4.184 mmol, 5.0 eq) dropwise into the reaction mixture and the resulting mixture was stirred at room temperature for 2 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.4% methanol in dichloromethane) to obtain 0.121 g of methyl 2-(3-(2,4-dimethoxyphenyl)-1-methylureido)-5-oxo-5H-thieno[3,2-b]pyran-6-carboxylate (9); Yield: 34.59%; MS (ES): m/z 418.42 [M+H]⁺; ¹H NMR (DMSO-d6, 400 MHZ): 8.85 (s, 1H), 8.10-8.09 (d, J=4 Hz, 2H), 7.82 (s, 1H), 6.62 (s, 1H), 6.49-6.46 (m, 1H), 3.90 (s, 3H), 3.80 (s, 3H), 3.65 (s, 3H), 3.35 (s, 3H).

### Step 10

A three-necked RBF was charged with Intermediate (9) (0.121 g, 0.289 mmol, 1.0 eq) in dichloromethane (20 mL), and trimethylsilyl iodide (0.2 mL, 1.44 mmol, 5.0 eq) at room temperature. The reaction mixture was stirred for 16 h at that temperature. After completion of reaction the mixture was concentrated under reduced pressure to obtain the crude material which was triturated with methanol to obtain 0.103 g of 2-(3-(2,4-dimethoxyphenyl)-1-methylureido)-5-oxo-5H-thieno[3,2-b]pyran-6-carboxylic acid (Example 14); Yield: 88.08%); MS (ES): m/z 404.39 [M+H]⁺; ¹H NMR (DMSO-d6, 400 MHZ): 12.484 (s, 1H), 8.844 (s, 1H), 8.8775 (s, 1H), 7.252-7.231 (d, J=8.4 Hz, 1H), 6.898 (s, 1H), 6.665-6.659 (d, J=2.4, 1H), 6.552-6.525 (m, 1H), 3.796 (s, 3H), 3.787 (s, 3H), 3.579 (S, 3H).

### Example 15

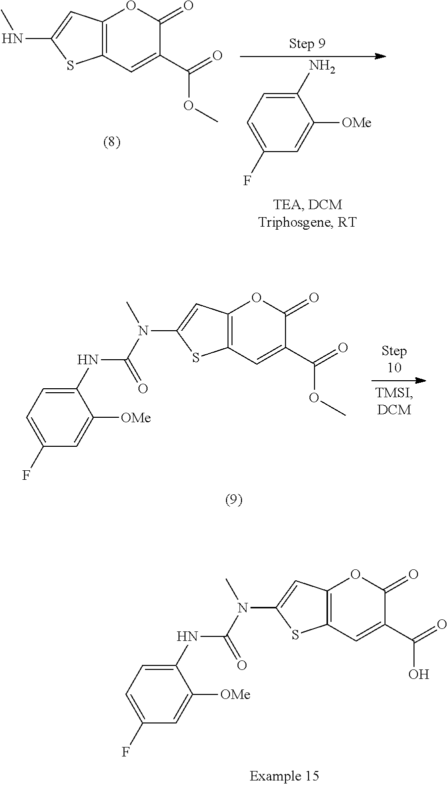

### Step 9

A three-necked RBF was charged with a solution of 4-fluoro-2-methoxyaniline (0.170 g, 0.627 mmol, 1.5 eq) in dichloromethane (30 mL). Triphosgene (0.065 g, 0.219 mmol, 0.35 eq) was added at 0° C. After 15 min, Intermediate (8) from Scheme 4 (0.255 g, 1.0626 mmol, 0.3 eq) was added followed by triethylamine (0.253 g, 1.2552 mmol, 3.0 eq) dropwise into the reaction mixture and the mixture was stirred at room temperature for 2 h. After completion of reaction the mixture was transferred into water and extracted with dichloromethane. Organic layers were combined, washed with brine solution, dried over anhydrous sodium sulphate and concentrated under reduced to obtain the crude material. The crude was purified by silica gel column chromatography (2.3% methanol in dichloromethane) to obtain 0.142 g of methyl 2-(3-(4-fluoro-2-methoxyphenyl)-1-methylureido)-5-oxo-5H-thieno[3,2-b]pyran-6-carboxylate (9); Yield: 32.78%; MS (ES):407.38 m/z [M+H]⁺; LCMS purity: 96.07%; ¹H NMR (DMSO-d6, 400 MHZ): 8.82 (s, 1H), 8.10 (s, 1H), 7.85 (s, 1H), 7.63-7.61 (t, J=8.8 Hz, 1H), 7.23-7.20 (m, 1H), 6.89-6.85 (m, 1H), 3.84 (s, 3H), 3.75 (s, 3H), 3.29 (s, 3H).

### Step 10

A three-necked RBF was charged with Intermediate (9) (0.107 g, 0.359 mmol, 1.0 eq) in dichloromethane (20 mL), and trimethylsilyl iodide (0.3 mL, 1.293 mmol, 5.0 eq) at room temperature. The reaction mixture was stirred for 16 h at that temperature. After completion of reaction, the mixture was concentrated under reduced pressure to obtain the crude material which was triturated with methanol to obtain 0.107 g of 2-(3-(4-fluoro-2-methoxyphenyl)-1-methylureido)-5-oxo-5H-thieno[3,2-b]pyran-6-carboxylic acid (Example 15); Yield: 78.05%; MS (ES): 393.2 m/z [M+H]⁺; ¹H NMR (DMSO-d6, 400 MHZ): 12.50 (s, 1H), 9.95 (s, 1H), 8.78 (s, 1H), 7.40-7.438 (t, J=8.8 Hz, 1H), 7.05-7.03 (d, J=8 Hz, 1H), 6.92 (s, 1H), 6.80 (s, 1H), 3.78 (s, 3H), 3.59 (s, 3H).

### Example 16

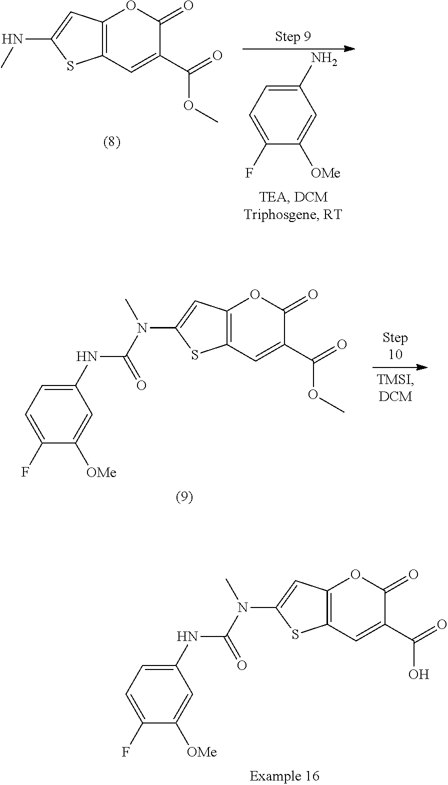

### Step 9

A three-necked RBF was charged with a solution of 4-fluoro-3-methoxyaniline (0.5 g, 3.54 mmol, 1.0 eq) in dichloromethane (30 mL). Triphosgene was added (0.370 g, 1.24 mmol, 0.35 eq) at 0° C. After 15 min Intermediate (8) from Scheme 4 (0.254 g, 1.06 mmol, 0.3 eq) was added followed by triethylamine (1.6 mL, 10.63 mmol, 3.0 eq) dropwise into the reaction mixture and stirred at room temperature for 3 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.8% methanol in dichloromethane) to obtain 0.121 g of methyl 2-(3-(4-fluoro-3-methoxyphenyl)-1-methylureido)-5-oxo-5H-thieno[3,2-b]pyran-6-carboxylate (9); Yield: 28.05%; MS (ES): m/z 407.38 [M+H]⁺; ¹H NMR (DMSO-d6, 400 MHZ): 8.85 (s, 1H), 8.10-8.09 (d, J=4 MHz, 1H), 7.83 (s, 1H), 7.30-7.28 (t, J=8 MHz, 3H), 3.81 (s, 3H), 3.71 (s, 3H), 3.29 (s, 3H).

### Step 10

A three-necked RBF was charged with Intermediate (9) (0.120 g, 0.295 mmol, 1.0 eq) in dichloromethane (25 mL), and trimethylsilyl iodide (0.26 mL, 1.47 mmol, 5.0 eq) at room temperature. The reaction mixture was stirred for 16 h at that temperature. After completion of reaction the mixture was concentrated under reduced pressure to obtain the crude material which was triturated with methanol to obtain 0.079 g of 2-(3-(4-fluoro-3-methoxyphenyl)-1-methylureido)-5-oxo-5H-thieno[3,2-b]pyran-6-carboxylic acid (Example 16); Yield: 68.19%; MS (ES): m/z 392.15 [M+H]⁺, ¹H NMR (DMSO-d6, 400 MHZ): 12.55 (s, 1H), 9.44 (s, 1H), 8.79-8.77 (d, J=8 MHz, 1H), 7.38-7.36 (d, J=8 MHz, 1H), 7.21-7.19 (t, J=8 MHz, 1H), 7.11 (s, 1H), 6.90 (s, 1H), 3.83 (s, 3H), 3.59 (s, 3H).

### Example 17

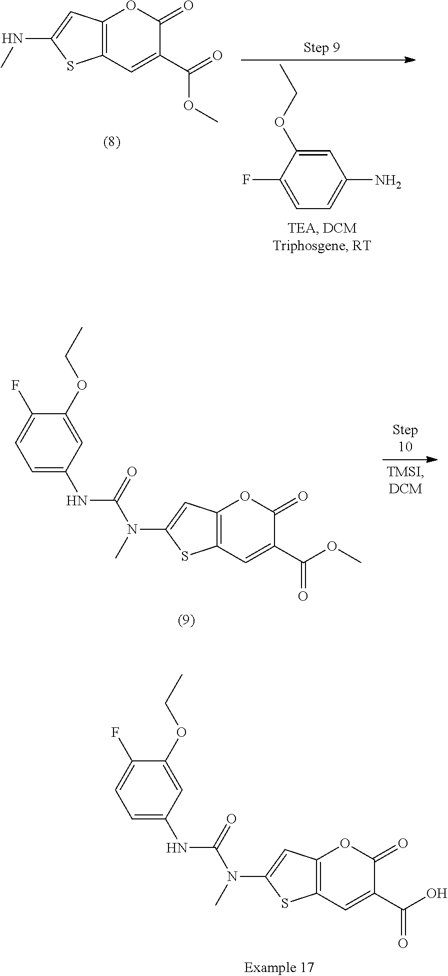

### Step 9

A three-necked RBF was charged with a solution of 3-ethoxy-4-fluoroaniline (0.3 g, 1.93 mmol, 2.1 eq) in dichloromethane (30 mL). Triphosgene (0.2 g, 0.6755 mmol, 0.35 eq) was added at 0° C. After 15 min, Intermediate (8) from Scheme 4 (0.22 g, 0.9205 mmol, 1.0 eq) was added followed by triethylamine (0.975, 9.65 mmol, 5.0 eq) dropwise into reaction mixture and stirred at room temperature for 2 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.5% methanol in dichloromethane) to obtain 0.12 g of methyl 2-(3-(3-ethoxy-4-fluorophenyl)-1-methylureido)-5-oxo-5H-thieno[3,2-b]pyran-6-carboxylate (9); Yield: 31.04%; MS (ES): m/z 420.41[M+H]⁺; ¹H NMR (DMSO-d6, 400 MHZ): 8.886 (s, 1H), 8.196 (s, 1H), 7.889 (s, 1H), 7.775-7.432 (m, 3H), 4.156-4.010 (m, 2H), 3.796 (s, 3H), 3.321 (s, 3H), 1.396-1.214 (m, 3H).

### Step 10

A three-necked RBF was charged with Intermediate (9) (0.12 g, 0.2857 mmol, 1.0 eq) in dichloromethane (80 mL), and trimethylsilyl iodide (0.2 mL, 1.428 mmol, 5 eq) at room temperature. The reaction mixture was stirred for 16 h at that temperature. After completion of reaction, the mixture was concentrated under reduced pressure to obtain the crude material which was triturated with methanol to obtain 0.078 g of 2-(3-(3-ethoxy-4-fluorophenyl)-1-methylureido)-5-oxo-5H-thieno[3,2-b]pyran-6-carboxylic acid (Example 17); Yield: 81.17%; MS (ES): m/z 406.38 [M+H]⁺; ¹H NMR (DMSO-d6, 400 MHZ): 9.441 (s, 1H), 8.802 (s, 1H), 7.367-7.351 (d, J=6.4 MHz, 1H), 7.321-7.162 (t, J=8.8 MHz, 1H), 7.096 (s, 1H), 6.994 (s, 1H), 4.115-4.080 (t, J=6.8 MHz, 2H), 3.599 (s, 3H), 1.393-1.358 (t, J=6.8 MHz, 3H).

### Example 18

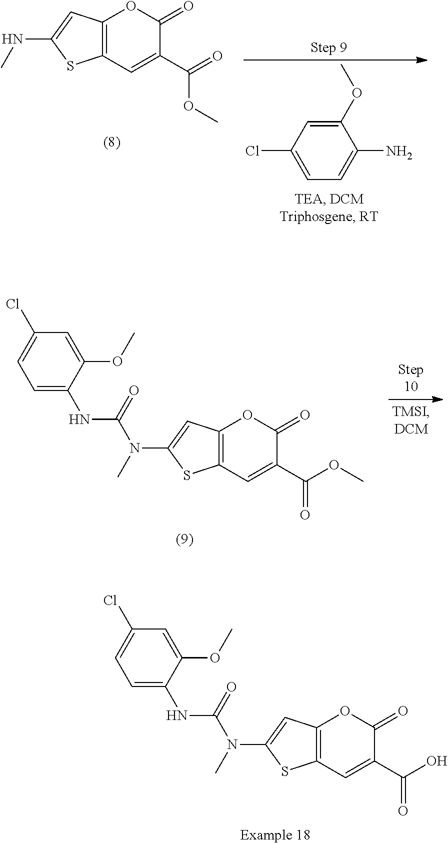

### Step 9

A three-necked RBF was charged with a solution of 4-chloro-2-methoxyaniline (0.197 g, 1.2552 mmol, 1.5 eq) in dichloromethane (30 mL). Triphosgene (0.130 g, 0.4391 mmol, 0.35 eq) was added at 0° C. After 15 min, Intermediate (8) from Scheme 4 (0.2 g, 0.836 mmol, 1.0 eq) was added followed by triethylamine (0.633, 6.276 mmol, 5.0 eq) dropwise into reaction mixture and stirred at room temperature for 2 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (1.9% methanol in dichloromethane) to obtain 0.140 g of methyl 2-(3-(4-chloro-2-methoxyphenyl)-1-methylureido)-5-oxo-5H-thieno[3,2-b]pyran-6-carboxylate (9); Yield: 39.61%; MS (ES): m/z 422.84 [M+H]⁺; ¹H NMR (DMSO-d6, 400 MHZ): 8.889 (s, 1H), 8.196 (s, 1H), 7.889 (s, 1H), 7.765-7.745 (d, J=8 Hz, 1H), 7.389 (s, 1H), 7.156-7.148 (d, J=5.2 Hz, 1H), 3.996 (s, 3H), 3.881 (s, 3H), 3.196 (s, 3H).

### Step 10

A three-necked RBF was charged with Intermediate (9) (0.14 g, 0.331 mmol, 1.0 eq) in dichloromethane (20 mL) and trimethylsilyl iodide (0.23 mL, 1.67 mmol, 5.0 eq) at room temperature. The reaction mixture was stirred for 16 h at that temperature. After completion of reaction the mixture was concentrated under reduced pressure to obtain the crude material which was triturated with methanol to obtain 0.096 g of 2-(3-(4-chloro-2-methoxyphenyl)-1-methylureido)-5-oxo-5H-thieno[3,2-b]pyran-6-carboxylic acid (Example 18); Yield: 70.92%; MS (ES): m/z 408.81[M+H]⁺; ¹H NMR (DMSO-d6, 400 MHZ): 12.504 (s, 1H), 8.491 (s, 1H), 8.787 (s, 1H), 7.469-7.448 (d, J=8.4 MHz, 1H), 7.197 (s, 1H), 7.048-7.027 (d, J=8.4 MHz, 1H), 6.933 (s, 1H), 3.853 (s, 3H), 3.594 (s, 3H).

### Example 19

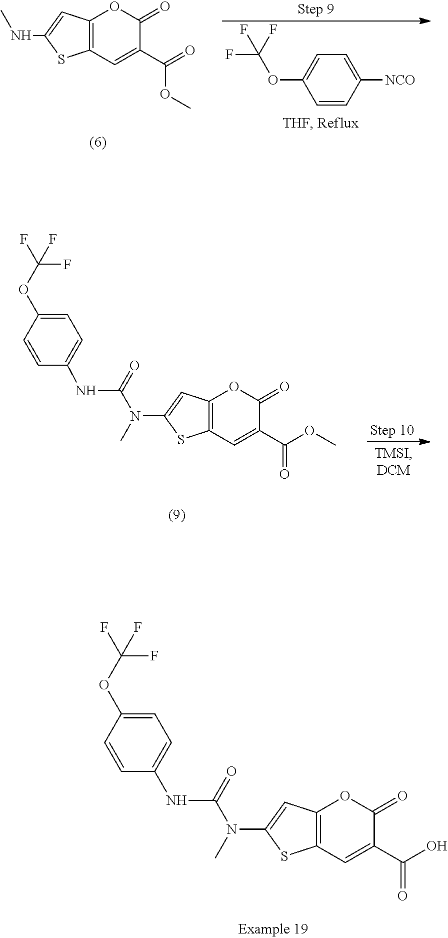

### Step 9

A three-necked RBF was charged with a solution of Intermediate (8) (0.300 g, 1.25 mmol, 1.0 eq) in tetrahydrofuran (25 mL), and added 1-isocyanato-4-(trifluoromethoxy)benzene (0.331 g, 1.88 mmol, 1.5 eq). The reaction mixture was stirred it at 90-100° C. for 16 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.170 g of methyl 2-(1-methyl-3-(4-(trifluoromethoxy)phenyl)ureido)-5-oxo-5H-thieno[3,2-b]pyran-6-carboxylate (9); Yield: 30.65%; MS (ES): m/z 442.37 [M+H]; ¹H NMR (DMSO-d6, 400 MHZ): 8.970 (s, 1H), 8.210 (s, 1H), 7.986 (s, 1H), 7.328-7.221 (m, 2H), 6.996-6.834 (m, 2H), 3.802 (s, 3H) 3.389 (s, 3H).

### Step 10

A three-necked RBF was charged with Intermediate (9) (0.170 g, 0.384 mmol, 1.0 eq) in dichloromethane (20 mL), and trimethylsilyl iodide (0.27 ml, 1.92 mmol, 5.0 eq) at room temperature. The reaction mixture was stirred for 16 h at that temperature. After completion of reaction, the mixture was concentrated under reduced pressure to obtain the crude material which was triturated with methanol to obtain 0.105 g of 2-(1-methyl-3-(4-(trifluoromethoxy)phenyl)ureido)-5-oxo-5H-thieno[3,2-b]pyran-6-carboxylic acid (Example 19); Yield: 63.79%; MS (ES): m/z 428.34 [M+H]⁺; ¹H NMR (DMSO-d6, 400 MHZ): 12.491 (s, 1H), 9.642 (s, 1H), 8.826 (s, 1H), 7.671-7.648 (d, J=8.8 Hz, 2H), 7.396-7.374 (d, J=8.8 Hz, 2H), 6.976 (s, 1H) 3.624 (s, 3H).

### Example 20

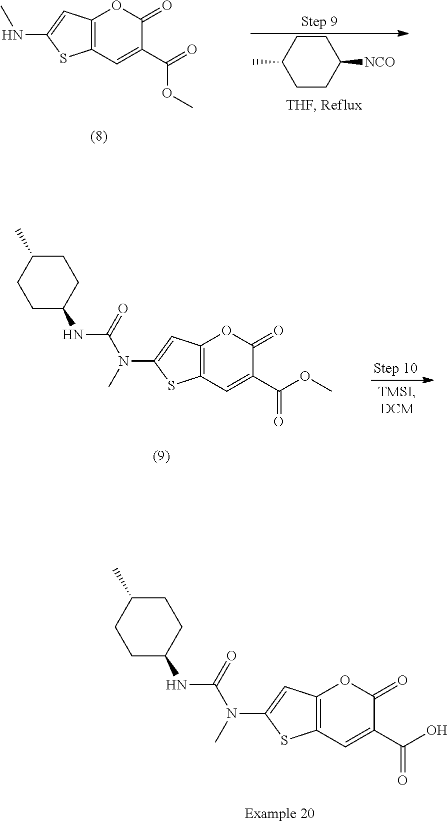

### Step 9

A three-necked RBF was charged with a solution of Intermediate (8) (0.250 g, 1.04 mmol, 1.0 eq) in tetrahydrofuran (25 mL), and (1R, 4R)-1-isocyanato-4-methylcyclohexane (0.727 g, 5.22 mmol, 5 eq). The reaction mixture was stirred at 90-100° C. for 16 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.6% methanol in dichloromethane) to obtain 0.125 g of methyl 2-(1-methyl-3-((1r,4r)-4-methylcyclohexyl)ureido)-5-oxo-5H-thieno[3,2-b]pyran-6-carboxylate (9); Yield: 32.37%; MS (ES): m/z 379.44 [M+H]; H NMR (DMSO-d6, 400 MHZ): 7.996 (s, 1H), 7.775 (s, 1H), 7.102 (s, 1H), 3.696 (s, 3H), 3.449 (m, 1H), 3.201 (s, 3H), 1.881-1.502 (m, 4H), 1.496-1.392 (m, 5H), 0.912-0.882 (d, J=8 Hz, 3H).

### Step 10

A three-necked RBF was charged with Intermediate (9) (0.125 g, 0.330 mmol, 1.0 eq) in dichloromethane (20 mL), and trimethylsilyl iodide (0.24 ml, 1.653 mmol, 5.0 eq) at room temperature. The reaction mixture was stirred for 16 h at that temperature. After completion of reaction, the mixture was concentrated under reduced pressure to obtain the crude material which was triturated with methanol to obtain 0.098 g of 2-(1-methyl-3-((1r,4r)-4-methylcyclohexyl)ureido)-5-oxo-5H-thieno[3,2-b]pyran-6-carboxylic acid (Example 20); Yield: 81.43%; MS (ES): m/z 364.42 [M+H]⁺; ¹H NMR (DMSO-d6, 400 MHZ): 12.49 (s, 1H), 8.76 (s, 1H), 7.38-7.36 (d, J=8 Hz, 1H), 6.80 (s, 1H), 3.56 (s, 3H), 1.84-1.81 (m, 1H), 1.76-1.72 (m, 2H), 1.37-1.32 (m, 3H), 1.05-0.99 (m, 3H), 0.90-0.87 (m, 4H).

### Example 21

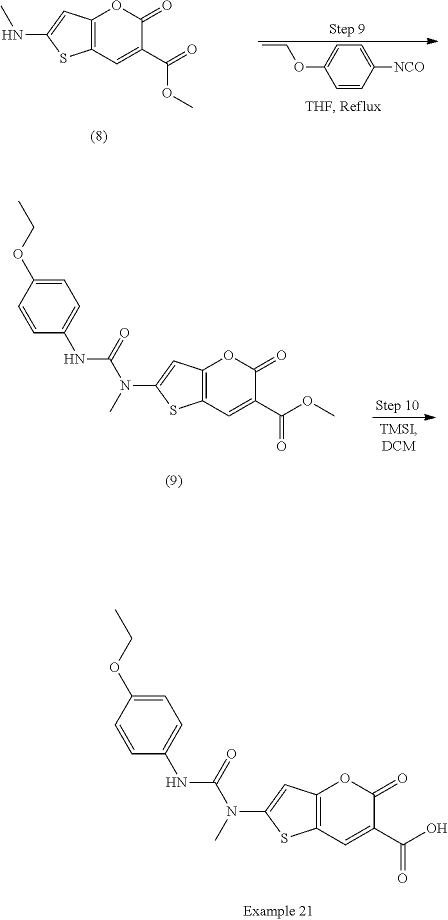

### Step 9

A three-necked RBF was charged with a solution of Intermediate (8) (0.250 g, 1.046 mmol, 1.0 eq) in tetrahydrofuran (25 mL) and was added 1-ethoxy-4-isocyanatobenzene (0.852 g, 5.23 mmol, 5 eq). The reaction mixture stirred it at 90-100° C. for 16 h. After completion of reaction, the mixture was transferred into water and extracted with dichloromethane. Organic layers were combined, washed with brine solution, dried over anhydrous sodium sulphate and concentrated under reduced pressure to obtain the crude material. The crude was further purified by silica gel column chromatography (2.1% methanol in dichloromethane) to obtain 0.095 g of methyl 2-(3-(4-ethoxyphenyl)-1-methylureido)-5-oxo-5H-thieno[3,2-b]pyran-6-carboxylate (9); Yield: 22.59%; MS (ES): m/z 402.42 [M+H]⁺; ¹H NMR (DMSO-d6, 400 MHZ): 8.970 (s, 1H), 8.210 (s, 1H), 7.986 (s, 1H), 7.328-7.221 (m, 2H), 6.996-6.834 (m, 2H), 4.118-4.015 (m, 2H), 3.802 (s, 3H) 3.389 (s, 3H), 1.412-1.318 (m, 3H).

### Step 10

A three-necked RBF was charged with Intermediate (9) (0.095 g, 0.236 mmol, 1.0 eq) in dichloromethane (20 mL), and trimethylsilyl iodide (0.17 ml, 1.18 mmol, 5.0 eq) at room temperature. The reaction mixture was stirred for 16 h at that temperature. After completion of reaction the mixture was concentrated under reduced pressure to obtain the crude material which was triturated with methanol to obtain 0.032 g of 2-(3-(4-ethoxyphenyl)-1-methylureido)-5-oxo-5H-thieno[3,2-b]pyran-6-carboxylic acid (Example 21); Yield: 34.90%; MS (ES): m/z 388.39 [M+H]⁺; ¹H NMR (DMSO-d6, 400 MHZ): 12.490 (s, 1H), 8.334 (s, 1H), 7.291-7.270 (d, J=8.4 Hz, 1H), 6.810-6.789 (d, J=8.4 Hz, 1H), 6.098 (s, 1H), 5.876 (s, 1H), 5.725 (s, 1H), 3.981-3.931 (m, 2H), 2.948 (s, 3H), 1.323-1.288 (t, J=6.8 Hz, 3H).

### Example 22

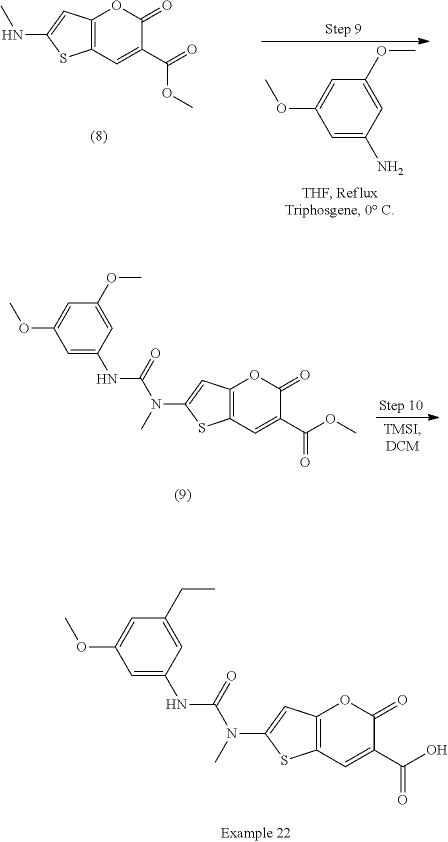

### Step 9

A three-necked RBF was charged with a solution of 3,5-dimethoxyaniline (0.240 g, 1.5673 mmol, 1.50 eq) in dichloromethane (30 mL). Triphosgene (0.150 g, 0.5485 mmol, 0.35 eq) was added at 0° C. After 15 min, Intermediate (8) from Scheme 4 (0.220 g, 0.9205 mmol, 1.0 eq) was added followed by triethylamine (0.7 mL, 5.2245 mmol, 5.0 eq) dropwise into reaction mixture and stirred at room temperature for 2 h. After completion of reaction the mixture was transferred into water and extracted with dichloromethane. Organic layers were combined, washed with brine solution, dried over anhydrous sodium sulphate and concentrated under reduced pressure to obtain the crude material. The crude was purified by silica gel column chromatography (2.2% methanol in dichloromethane) to obtain 0.123 g of methyl 2-(3-(3,5-dimethoxyphenyl)-1-methylureido)-5-oxo-5H-thieno[3,2-b]pyran-6-carboxylate (9); Yield:28.59%; MS (ES): m/z 419.42 [M+H]⁺; ¹H NMR (DMSO-d6, 400 MHZ):8.969 (s, 1H), 8.156 (s, 1H), 7.969 (s, 1H), 6.969 (s, 2H), 6.126 (s, 1H), 3.969 (s, 6H), 3.812 (s, 3H), 3.126 (s, 3H).

### Step 10

A three-necked RBF was charged with Intermediate (9) (0.120 g, 0.2867 mmol, 1.0 eq) in dichloromethane (20 mL) and trimethylsilyl iodide (0.22 mL, 1.4335 mmol, 5.0 eq) at room temperatures. The reaction mixture was stirred for 16 h at that temperature. After completion of reaction the mixture was concentrated under reduced pressure to obtain the crude material which was triturated with methanol to obtain 0.101 g of 2-(3-(3,5-dimethoxyphenyl)-1-methylureido)-5-oxo-5H-thieno[3,2-b]pyran-6-carboxylic acid (Example 22); Yield: 87.90%); MS (ES): m/z 405.39[M+H]⁺; ¹H NMR (DMSO-d6, 400 MHZ):12.541 (s, 1H), 9.369 (s, 1H), 8.807 (s, 1H), 6.942 (s, 1H), 6.815 (s, 1H), 6.276 (s, 1H), 3.741 (s, 5H), 3.595 (s, 1H).

### Example 23

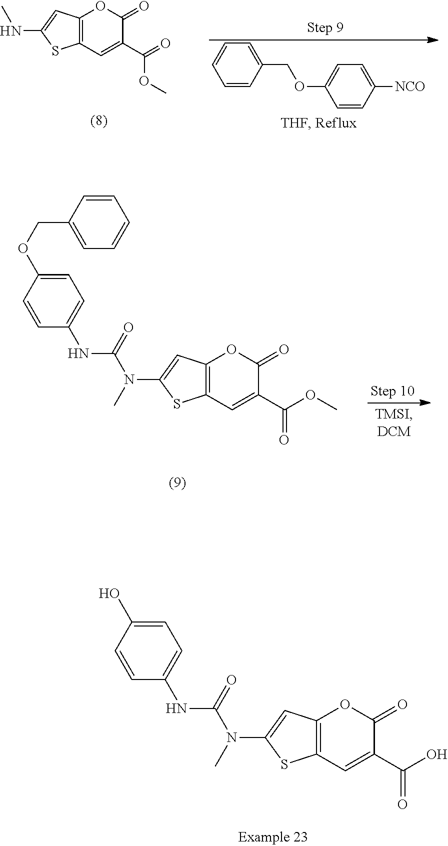

### Step 9

A three-necked RBF was charged with Intermediate (8) from Scheme 4 (0.250 g, 1.046 mmol, 1.0 eq) in tetrahydrofuran (25 mL), and 1-(benzyloxy)-4-isocyanatobenzene (1.176 g, 5.23 mmol, 5 eq). The reaction mixture was stirred it at 90-100° C. for 16 h. After completion of reaction, the mixture was transferred into water and extracted with dichloromethane. Organic layers were combined, washed with brine, dried over anhydrous sodium sulphate and concentrated under reduced pressure to obtain the crude material. The crude was purified by silica gel column chromatography (2.4% methanol in dichloromethane) to obtain 0.143 g of methyl 2-(3-(4-(benzyloxy)phenyl)-1-methylureido)-5-oxo-5H-thieno[3,2-b]pyran-6-carboxylate (9); Yield:29.46%; MS (ES): m/z 464.49[M+H]⁺; ¹H NMR (DMSO-d6, 400 MHZ): 9.316 (s, 1H), 9.250 (s, 1H), 8.785 (s, 1H), 7.512-7.428 (m, 3H), 7.401-7.321 (m, 2H), 7.156-7.6996 (m, 2H), 6.881-6.712 (m, 2H), 5.181 (s, 2H), 3.591 (s, 3H), 3.339 (s, 3H).

### Step 10

A three-necked RBF was charged with Intermediate (9) (0.143 g, 0.3078 mmol, 1.0 eq) in dichloromethane (20 mL), and trimethylsilyl iodide (0.22 ml, 1.539 mmol, 5.0 eq) at room temperature. The reaction mixture was stirred for 16 h at that temperature. After completion of reaction, the mixture was concentrated under reduced pressure to obtain the crude material which was triturated with methanol to obtain 0.031 g of 2-(3-(4-hydroxyphenyl)-1-methylureido)-5-oxo-5H-thieno[3,2-b]pyran-6-carboxylic acid (Example 23); Yield: 29.94%; MS (ES): m/z 360.34 [M+H]⁺; ¹H NMR (DMSO-d6, 400 MHZ): 12.490 (s, 1H), 9.316 (s, 1H), 9.250 (s, 1H), 8.785 (s, 1H), 7.260-7.250 (d, J=7.2 Hz, 2H), 6.909 (s, 1H), 6.755-6.737 (d, J=7.2 Hz, 2H), 3.574 (s, 3H).

### Example 24

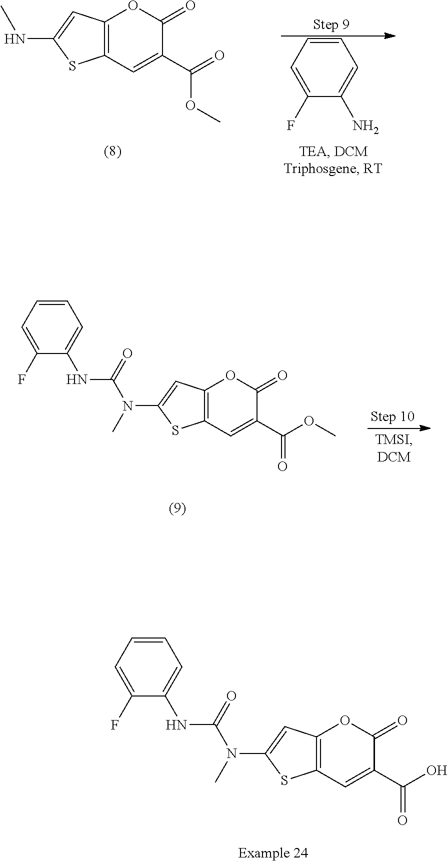

### Step 9

A three-necked RBF was charged with a solution of 2-fluoroaniline (0.25 g, 1.046 mmol, 1.0 eq) in dichloromethane (15 mL). Triphosgene (0.130 g, 0.418 mmol, 0.4 eq) was added at 0° C. After 15 min, Intermediate (8) (0.175 g, 1.569 mmol, 1.5 eq) was added followed by triethylamine (0.5 mL, 3.138 mmol, 3.0 eq) dropwise into the reaction mixture and stirred at room temperature for 2 h. After completion of reaction, the mixture was transferred into water and extracted with dichloromethane. Organic layers were combined, washed with brine, dried over anhydrous sodium sulphate and concentrated under reduced pressure to obtain the crude material. The crude was purified by silica gel column chromatography (2.2% methanol in dichloromethane) to obtain 0.140 g of methyl 2-(3-(2-fluorophenyl)-1-methylureido)-5-oxo-5H-thieno[3,2-b]pyran-6-carboxylate (9); Yield: 35.60%; MS (ES): m/z 377.06 [M+H]⁺; ¹H NMR (DMSO-d₆, 400 MHZ): 8.81 (s, 1H), 8.09 (s, 1H), 7.96-7.89 (m, 1H), 7.83-7.82 (t, J=4 Hz, 1H), 7.19-7.11 (m, 3H), 3.72 (s, 3H), 3.22 (s, 3H).

### Step 10

A three-necked RBF was charged with Intermediate (9) (0.140 g, 0.372 mmol, 1.0 eq) in dichloromethane (10 mL), and trimethylsilyl iodide (0.5 mL, 3.72 mmol, 10.0 eq) at room temperature. The reaction mixture was stirred for 16 h at that temperature. After completion of reaction the mixture was concentrated under reduced pressure to obtain the crude material which was triturated with methanol to obtain 0.105 g of 2-(3-(2-fluorophenyl)-1-methylureido)-5-oxo-5H-thieno[3,2-b]pyran-6-carboxylic acid (Example 24); Yield: 77.90%; MS (ES): m/z 363.00 [M+H]⁺; ¹H NMR (DMSO-d₆, 400 MHZ): 12.53 (s, 1H), 9.44 (s, 1H), 8.81 (s, 1H), 7.47-7.43 (t, J=8 Hz, 1H), 7.33-7.30 (m, 2H), 7.26-7.22 (m, 1H), 6.96 (s, 1H), 3.62 (s, 3H).

### Example 25

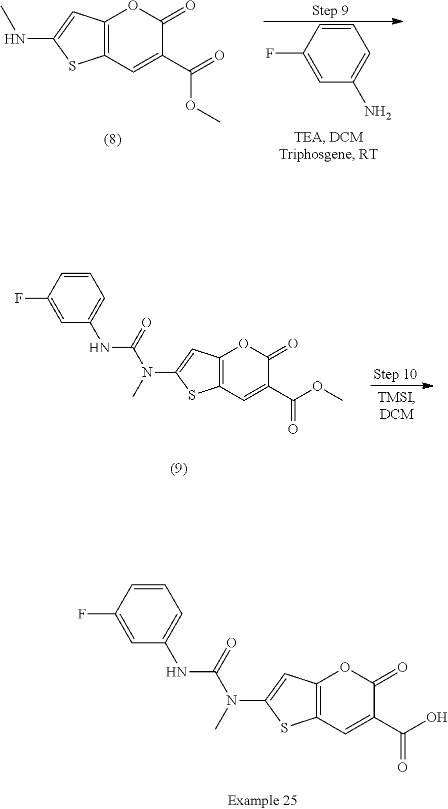

### Step 9

A three-necked RBF was charged with a solution of 3-fluoroaniline (0.175 g, 1.567 mmol, 1.50 eq) in dichloromethane (30 mL). Triphosgene (0.165 g, 0.5484 mmol, 0.35 eq) was added at 0° C. After 15 min, Intermediate (8) (0.220 g, 0.9205 mmol, 1.0 eq) was added followed by triethylamine (0.6 mL, 4.81 mmol, 5.0 eq) dropwise into the reaction mixture and stirred at room temperature for 2 h. After completion of reaction, the mixture was transferred into water and extracted with dichloromethane. Organic layers were combined, washed with brine, dried over anhydrous sodium sulphate and concentrated under reduced pressure to obtain the crude material. The crude was purified by silica gel column chromatography (2.2% methanol in dichloromethane) to obtain 0.155 g of methyl 2-(3-(3-fluorophenyl)-1-methylureido)-5-oxo-5H-thieno[3,2-b]pyran-6-carboxylate (9); Yield: 39.41%; MS (ES): m/z 377.36 [M+H]⁺; ¹H NMR (DMSO-d6, 400 MHZ): 8.80 (s, 1H), 8.09 (s, 1H), 7.83 (s, 1H), 7.74-7.71 (m, 1H), 7.40-7.35 (m, J=8 Hz, 2H), 6.90 (s, 1H), 3.69 (s, 3H), 3.29 (s, 3H).

### Step 10

A three-necked RBF was charged with Intermediate (9) (0.130 g, 0.305 mmol, 1.0 eq) in dichloromethane (20 mL), and trimethylsilyl iodide (0.3 mL, 1.5255 mmol, 5.0 eq) at room temperature. The reaction mixture was stirred for 16 h at that temperature. After completion of reaction the mixture was concentrated under reduced pressure to obtain the crude material which was triturated with methanol to obtain 0.105 g of 2-(3-(3-fluorophenyl)-1-methylureido)-5-oxo-5H-thieno[3,2-b]pyran-6-carboxylic acid (Example 25); Yield: 70.36%; MS (ES): m/z 363.33 [M+H]⁺; ¹H NMR (DMSO-d6, 400 MHZ): 12.546 (s, 1H), 9.619 (s, 1H), 8.820 (s, 1H), 7.476-7.384 (m, 3H), 6.968-6.948 (d, J=8 Hz, 2H), 3.611 (s, 3H).

### Example 26

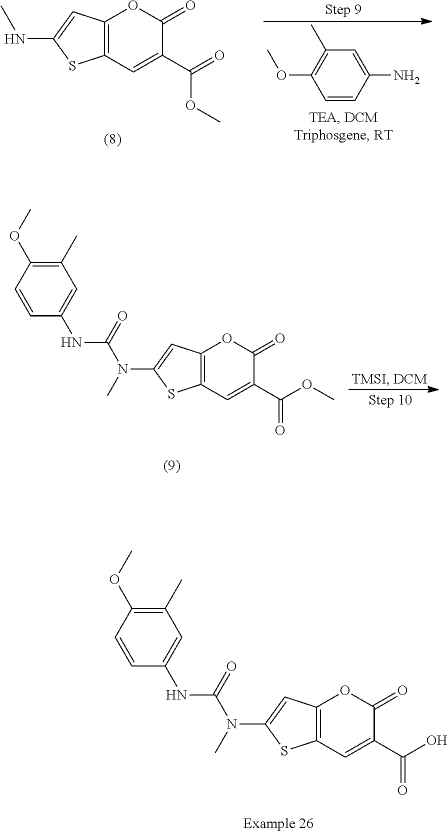

### Step 9

A three-necked RBF was charged with a solution of 4-methoxy-3-methylaniline (0.5 g, 3.64 mmol, 1.0 eq) in dichloromethane (30 mL). Triphosgene (0.37 g, 1.27 mmol, 0.35 eq) was added at 0° C. After 15 min, Intermediate (8) (0.17 g, 0.728 mmol, 0.2 eq) was added followed by triethylamine (2.5 mL, 18.2 mmol, 5.0 eq) dropwise into the reaction mixture and stirred at room temperature for 2 h. After completion of reaction, the mixture was transferred into water and extracted with dichloromethane. Organic layers were combined, washed with brine, dried over anhydrous sodium sulphate and concentrated under reduced pressure to obtain the crude material. The crude was purified by silica gel column chromatography (2.2% methanol in dichloromethane) to obtain 0.120 g of methyl 2-(3-(4-methoxy-3-methylphenyl)-1-methylureido)-5-oxo-5H-thieno[3,2-b]pyran-6-carboxylate (9); Yield: 29.36%; MS (ES): m/z 402.42[M+H]⁺; ¹H NMR (DMSO-d6, 400 MHZ): 8.991 (s, 1H), 8.210 (s, 1H), 7.996 (s, 1H), 7.892 (s, 1H), 7.556-7.534 (d, J=8.8 Hz, 1H), 7.213-6.884 (m, 1H), 3.889 (s, 6H), 3.412 (s, 3H), 2.098 (s, 3H).

### Step 10

A three-necked RBF was charged with Intermediate (9) (0.120 g, 0.298 mmol, 1.0 eq) in dichloromethane (20 mL) and trimethylsilyl iodide (0.29 g, 1.49 mmol, 5.0 eq) at room temperature. The reaction mixture was stirred for 16 h at that temperature. After completion of reaction, the mixture was concentrated under reduced pressure to obtain the crude material which was triturated with methanol and purified by preparative HPLC (0.1% formic acid water in 100% acetonitrile) to obtain a fraction which was lyophilized to afford 0.015 g of 2-(3-(4-methoxy-3-methylphenyl)-1-methylureido)-5-oxo-5H-thieno[3,2-b]pyran-6-carboxylic acid (Example 26); Yield: 12.95%; MS (ES): m/z 388.39 [M+H]⁺, ¹H NMR (DMSO-d6, 400 MHZ): 12.497 (s, 1H), 9.205-9.191 (d, J=5.6, 1H), 8.795-8.770 (d, J=10, 1H), 7.215-7.181 (m, 1H), 7.111-7.088 (m, 1H), 6.960-6.869 (m, 1H), 6.764-6.742 (m, 1H), 3.766 (s, 3H), 3.579 (s, 3H), 2.136 (s, 3H).

### Example 27

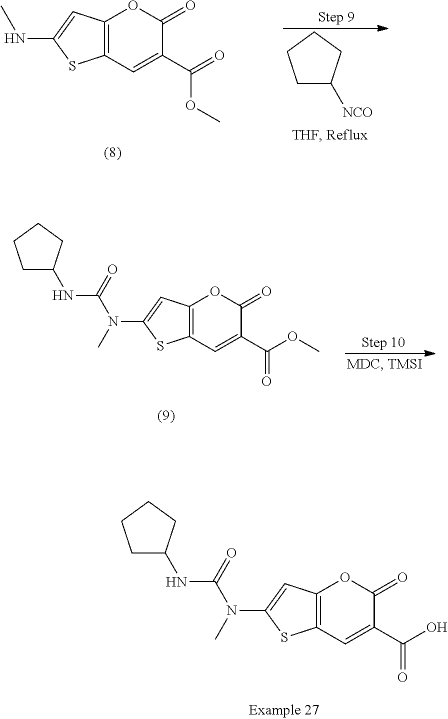

### Step 9

A three-necked RBF was charged with Intermediate (8) from Scheme 4 (0.250 g, 1.046 mmol, 1.0 eq) in tetrahydrofuran (25 mL), and isocyanatocyclopentane (1.176 g, 5.23 mmol, 5 eq). The reaction mixture stirred at 90-100° C. for 16 h. After completion of reaction the mixture was transferred into water and extracted with dichloromethane. Organic layers were combined, washed with brine, dried over anhydrous sodium sulphate and concentrated under reduced pressure to obtain the crude material. The crude was purified by silica gel column chromatography (2.1% methanol in dichloromethane) to obtain 0.18 g of methyl 2-(3-cyclopentyl-1-methylureido)-5-oxo-5H-thieno[3,2-b]pyran-6-carboxylate (9); Yield: 49.16%; MS (ES): m/z 350.36 [M+H]⁺; ¹H NMR (DMSO-d6, 400 MHZ): 8.757 (s, 1H), 7.775 (s, 1H), 6.796 (s, 1H), 3.696 (s, 3H), 3.591 (m, 1H), 3.339 (s, 3H), 1.887 (s, 2H), 1.692 (s, 2H), 1.528 (s, 4H).

### Step 10

A three-necked RBF was charged with Intermediate (9) (0.18 g, 0.5137 mmol, 1.0 eq) in dichloromethane (20 mL) and trimethylsilyl iodide (0.5137 g, 2.5684 mmol, 5.0 eq) at room temperature. The reaction mixture was stirred for 16 h at that temperature. After completion of reaction the mixture was concentrated under reduced pressure to obtain the crude material which was triturated with methanol to obtain 0.095 g of 2-(3-cyclopentyl-1-methylureido)-5-oxo-5H-thieno[3,2-b]pyran-6-carboxylic acid (Example 27); Yield: 54.98%; MS (ES): m/z 336.36 [M+H]⁺; ¹H NMR (DMSO-d6, 400 MHZ): 12.495 (s, 1H), 8.757 (s, 1H), 7.437-7.423 (d, J=5.6 Hz, 1H), 6.800 (s, 1H), 4.015-4.001 (d, J=5.6 Hz, 1H), 1.887 (s, 2H), 1.692 (s, 2H), 1.528 (s, 4H).

### Example 28

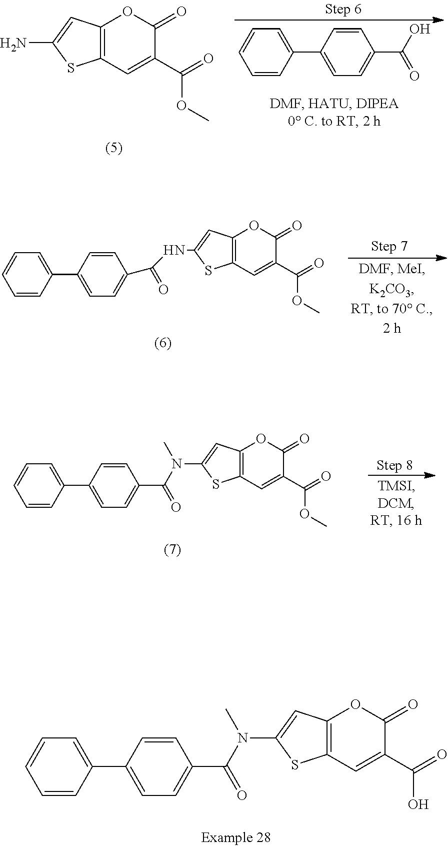

### Step 6

A three-necked RBF was charged with [1,1′-biphenyl]-4-carboxylic acid (0.275 g, 1.59 mmol, 1.0 eq) in N,N-dimethylformamide (12 mL), and was added 1-[bis(dimethylamino)methylene]-1H-1,2,3-triazolo[4,5-b]pyridinium 3-oxid hexafluorophosphate (0.911 g, 2.39 mmol, 1.5 eq) at 0° C. After 15 min, Intermediate (5) (0.287 g, 1.27 mmol, 0.8 eq) and N, N-diisopropylethylamine (0.8 mL, 4.79 mmol, 3.0 eq) were added into the reaction mixture and stirred at room temperature for 0.5 h. After completion of reaction, the mixture was transferred into water and extracted with ethyl acetate. Organic layers were combined, washed with brine solution, dried over anhydrous sodium sulphate and concentrated under reduced pressure to obtain the crude material. The crude was further purified by silica gel column chromatography (1.8% methanol in dichloromethane) to obtain 0.2 g of methyl 2-([1,1′-biphenyl]-4-carboxamido)-5-oxo-5H-thieno[3,2-b]pyran-6-carboxylate (6); Yield: 38.71%; MS (ES): m/z 405.42 [M+H]⁺; ¹H NMR (DMSO-d6, 400 MHZ): 10.2 (s, 1H), 7.68 (s, 1H), 7.62-7.60 (d, J=8 MHz, 2H), 7.31-7.24 (m, 5H), 7.20-7.12 (m, 3H), 3.62 (s, 3H).

### Step 7

A three-necked RBF was charged with Intermediate (6) (0.2 g, 0.493 mmol, 1.0 eq) in N,N-dimethylformamide (12 mL). To this solution was added potassium carbonate (K₂CO₃) (0.136 g, 0.986 mmol, 2.0 eq) followed by methyl iodide (0.70 g, 4.93 mmol, 10 eq) dropwise at 0° C. The reaction mixture was stirred at 70° C. for 2 h. After completion of reaction, the mixture was transferred into ice-cold water and the precipitate was filtered, washed with water and hexanes to obtain 0.205 g of 2-([1,1′-biphenyl]-4-carboxamido)-5-oxo-5H-thieno[3,2-b]pyran-6-carboxylate (7); Yield: 99.07%; MS (ES): m/z 419.45 [M+H]⁺; ¹H NMR (DMSO-d6, 400 MHZ): 7.89 (s, 1H), 7.65 (s, 2H), 7.43-7.32 (m, 5H), 7.21-7.12 (m, 3H), 3.65 (s, 3H), 3.26 (s, 3H).

### Step 8

A three-necked RBF was charged with Intermediate (7) (0.205 g, 0.488 mmol, 1.0 eq) in dichloromethane (12 mL). To this solution was added trimethylsilyl iodide (0.244 g, 1.22 mmol, 2.5 eq) and the reaction mixture was stirred for 16 h at room temperature. After completion of reaction, the mixture was concentrated under reduced pressure to obtain the crude material which was triturated with methanol to obtain 0.118 g of 2-(N-methyl-[1,1′-biphenyl]-4-carboxamido)-5-oxo-5H-thieno[3,2-b]pyran-6-carboxylic acid (Example 28); Yield: 59.55%; MS (ES): m/z 405.42[M+H]⁺; LCMS purity: 98.48%; HPLC purity: 95.43%; ¹H NMR (DMSO-d6, 400 MHZ): 12.71 (s, 1H), 8.90 (s, 1H), 8.29 (s, 1H), 8.09-8.02 (m, 3H), 7.75-7.73 (d, J=8.4 MHz, 1H), 7.67-7.64 (t, J=6 MHz, 2H), 7.19 (s, 1H), 3.58 (s, 3H).

### Example 29

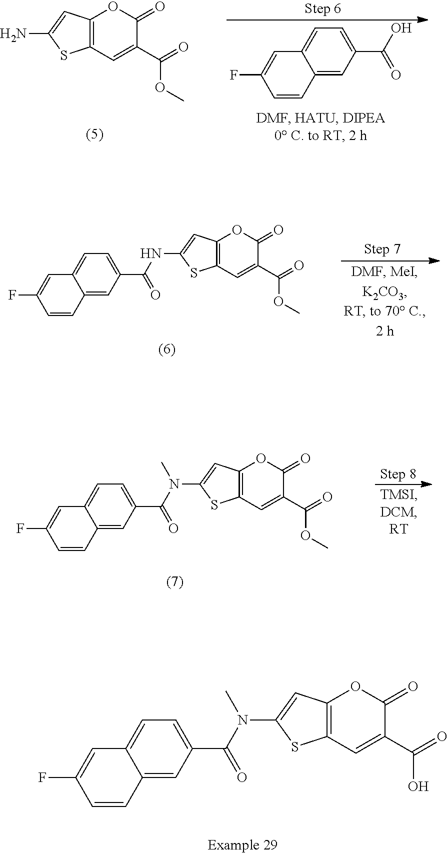

### Step 6

A three-necked RBF was charged with 6-fluoro-2-naphthoic acid (0.395 g, 2.08 mmol, 1.1 eq) in DMF (15 mL), and HATU (1.07 g, 2.83 mmol, 1.5 eq) at 0° C. After 15 min, Intermediate (5) (0.425 g, 1.89 mmol, 1.0 eq) was added into reaction mixture followed by N, N-diisopropylethylamine (0.73 g, 5.67 mmol, 3.0 eq) and the mixture was stirred at room temperature for 0.5 h. After completion of reaction the mixture was transferred into water and extracted with ethyl acetate. Organic layers were combined, washed with brine, dried over anhydrous sodium sulphate and concentrated under reduced pressure to obtain the crude material. The crude was purified by silica gel column chromatography (1.8% methanol in dichloromethane) to obtain 0.3 g of methyl 2-(6-fluoro-2-naphthamido)-5-oxo-5H-thieno[3,2-b]pyran-6-carboxylate (6); Yield: 40.01%; MS (ES): m/z 397.38 [M+H]⁺; ¹H NMR (DMSO-d6, 400 MHZ): 11.41 (s, 1H), 8.32 (s, 1H), 8.13-8.02 (m, 3H), 7.70 (s, 2H), 7.55 (s, 1H), 7.28-7.26 (d, J=8 MHz, 1H), 3.61 (s, 3H).

### Step 7

A mixture of Intermediate (6) (0.3 g, 0.755 mmol, 1.0 eq) in DMF (15 mL), and potassium carbonate (0.26 g, 1.88 mmol, 2.5 eq) was cooled to) ° C. and treated with methyl iodide (1.072 g, 7.55 mmol, 10 eq). After the addition was complete the mixture was heated to 70° C. and stirred at that temperature for 2 h. The mixture was transferred into ice-cold water, and the precipitate was filtered and washed with water and hexanes to obtain 0.310 g of methyl 2-(6-fluoro-N-methyl-2-naphthamido)-5-oxo-5H-thieno[3,2-b]pyran-6-carboxylate (7); Yield: 99.81%; MS (ES): m/z 411.40 [M+H]⁺; ¹H NMR (DMSO-d6, 400 MHZ): 8.33 (s, 1H), 8.11-8.02 (m, 3H), 7.79 (s, 2H), 7.55 (s, 1H), 7.315-7.302 (d, J=5.2 Hz, 1H), 3.58 (s, 3H), 3.20 (s, 3H).

### Step 8

A three-necked RBF was charged with Intermediate (7) (0.31 g, 0.753 mmol, 1.0 eq) in dichloromethane (15 mL) and trimethylsilyl iodide (0.376 g, 1.88 mmol, 2.5 eq) at room temperature. The reaction mixture was stirred for 16 h at that temperature. After completion of reaction the mixture was concentrated under reduced pressure to obtain the crude material which was triturated with methanol to obtain 0.125 g of 2-(6-fluoro-N-methyl-2-naphthamido)-5-oxo-5H-thieno[3,2-b]pyran-6-carboxylic acid (Example 29); Yield: 41.75%; MS (ES): m/z 405.42 [M+H]⁺; ¹H NMR (DMSO-d6, 400 MHZ): 12.69 (s, 1H), 8.92 (s, 1H), 8.36 (s, 1H), 8.19-8.16 (t, J=6 MHz, 1H), 8.09-8.07 (d, J=8.4 MHz, 1H), 7.87-7.79 (m, 2H), 7.59-7.55 (t, J=6.8 MHz, 1H), 7.21 (s, 1H), 3.58 (s, 3H).

### Example 30

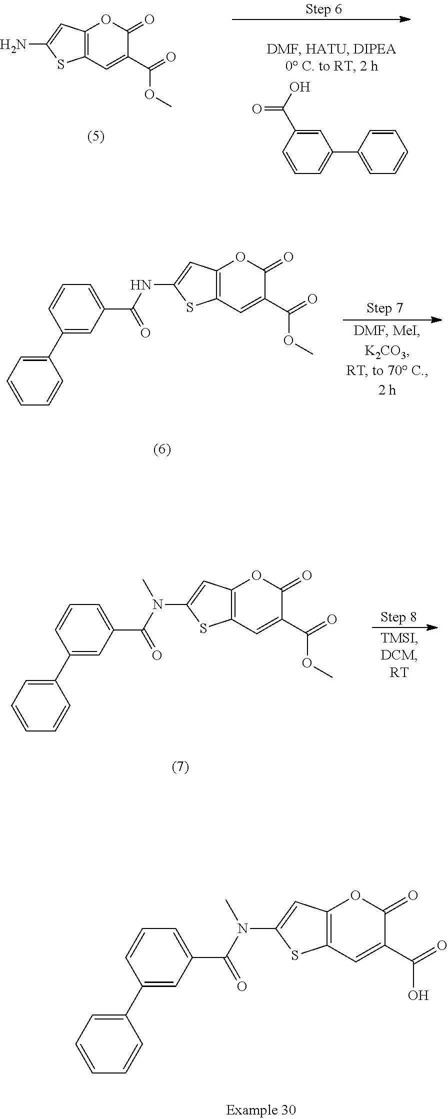

### Step 6

A three-necked RBF was charged with [1,1′-biphenyl]-3-carboxylic acid (0.2 g, 1.010 mmol, 1.0 eq) in DMF (10 mL) and cooled to 0° C. HATU (0.575 g, 1.5151 mmol, 1.5 eq) was added. After 15 min, Intermediate (5) (0.18 g, 0.808 mmol, 0.8 eq) was added followed by N, N-diisopropylethylamine (0.8 mL, 4.04 mmol, 4.0 eq) and the mixture was stirred at room temperature for 0.5 h. After completion of reaction, the mixture was transferred into water and extracted with ethylacetate. Organic layers were combined, washed with brine, dried over anhydrous sodium sulphate and concentrated under reduced pressure to obtain the crude material. The crude was further purified by silica gel column chromatography (1.8% methanol in dichloromethane) to obtain 0.150 g of methyl 2-([1,1′-biphenyl]-3-carboxamido)-5-oxo-SH-thieno[3,2-b]pyran-6-carboxylate (6); Yield: 46.29%; MS (ES): m/z 405.42 [M+H]⁺; ¹H NMR (DMSO-d6, 400 MHZ): 11.85 (s, 1H), 8.55 (s, 1H), 8.06 (s, 2H), 7.71-7.65 (m, 5H), 7.46-7.32 (m, 3H), 3.62 (s, 3H).

### Step 7

A three-necked RBF was charged with Intermediate (6) (0.150, 0.3699 mmol, 1.0 eq) in DMF (10 mL) and potassium carbonate (0.127 g, 0.9247 mmol, 2.5 eq) and cooled to 0° C. Methyl iodide (0.525 g, 3.703 mmol, 10.0 eq) was added dropwise. After the addition was complete the mixture was heated to 70° C. and stirred at that temperature for 2 h. After completion of reaction the mixture was poured into ice-cold water and the precipitate was filtered and washed with water and hexanes to obtain 0.120 g of methyl 2-(N-methyl-[1,1′-biphenyl]-3-carboxamido)-5-oxo-5H-thieno[3,2-b]pyran-6-carboxylate (7); Yield: 77.32%; MS (ES): m/z 419.45[M+H]⁺; ¹H NMR (DMSO-d6, 400 MHZ): 8.55 (s, 1H), 8.04 (s, 1H), 7.81 (s, 1H), 7.70-7.59 (m, 4H), 7.42-7.31 (m, 3H), 3.65 (s, 3H), 3.28 (s, 3H).

### Step 8

A three-necked RBF was charged with Intermediate (7) (0.120 g, 0.286 mmol, 1.0 eq) in dichloromethane (20 mL), and trimethylsilyl iodide (0.244 g, 1.22 mmol, 2.5 eq) at room temperature. The reaction mixture was stirred for 16 h at that temperature. After completion of reaction the mixture was concentrated under reduced pressure to obtain the crude material which was triturated with methanol to obtain 0.075 g of 2-(N-methyl-[1,1′-biphenyl]-3-carboxamido)-5-oxo-5H-thieno[3,2-b]pyran-6-carboxylic acid (Example 30); Yield: 64.66%; MS (ES): m/z 405.42[M+H]⁺; LCMS purity: 97.13%; HPLC purity: 96.37%; ¹H NMR (DMSO-d6, 400 MHZ): 12.644 (s, 1H), 8.90 (s, 1H), 7.952 (s, 1H) 7.898 (s, 1H), 7.761-7.743 (d, J=7.2 MHz, 2H), 7.649 (s, 2H), 7.517-7.482 (t, J=6.8 MHz, 2H), 7.430-7.413 (d, J=6.8 MHz, 1H), 7.163 (s, 1H), 3.555 (s, 3H).

### Example 31

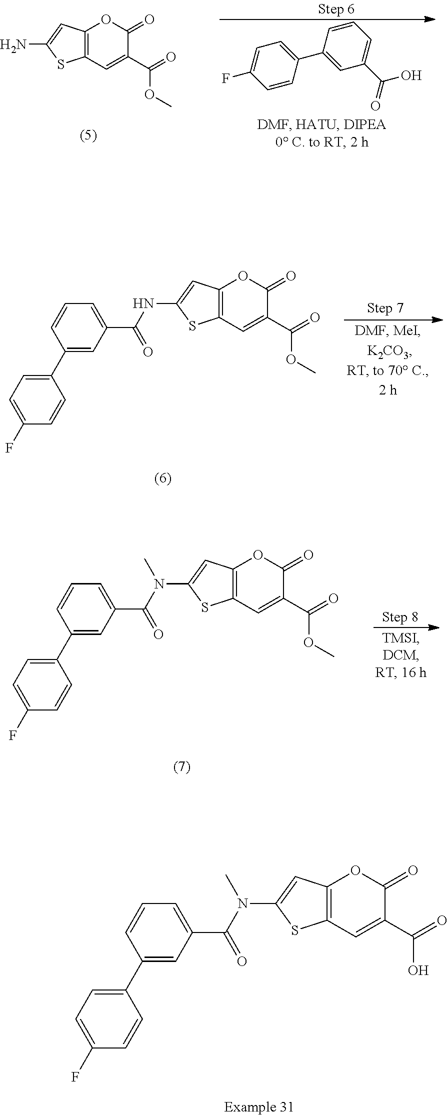

### Step 6

A three-necked RBF was charged with 4′-fluoro-[1,1′-biphenyl]-3-carboxylic acid (0.3 g, 1.3875 mmol, 1.0 eq) in DMF (10 mL) and cooled to 0° C. HATU was added and the mixture was stirred 15 min. Intermediate (5) (0.250 g, 1.11 mmol, 0.8 eq) and DIPEA (1.0 mL, 5.55 mmol, 4.0 eq) were added and the mixture was allowed to warm to room temperature and stirred for 0.5 h. After completion of reaction the mixture was transferred into water and extracted with ethyl acetate. Organic layers were combined, washed with brine, dried over anhydrous sodium sulphate and concentrated under reduced pressure to obtain the crude material. The crude was purified by silica gel column chromatography (2.8% methanol in dichloromethane) to obtain 0.220 g of methyl 2-(4′-fluoro-[1,1′-biphenyl]-3-carboxamido)-5-oxo-5H-thieno[3,2-b]pyran-6-carboxylate (6); Yield: 39.01%; MS (ES): m/z 424.41 [M+H]⁺; ¹H NMR (DMSO-d6, 400 MHZ): 11.21 (s, 1H), 8.54 (s, 1H), 8.03 (s, 2H), 7.81 (s, 1H), 7.68-7.60 (m, 2H), 7.47-7.46 (d, J=8 Hz 2H), 7.11-7.09 (d, J=8 Hz, 2H), 3.62 (s, 3H).

### Step 7

A three-necked RBF was charged with Intermediate (6) (0.220 g, 0.5195 mmol, 1.0 eq) in DMF (10 mL) and potassium carbonate (0.180 g, 1.3 mmol, 2.5 eq). The mixture was cooled to 0° C. and treated the dropwise addition of methyl iodide (0.370 g, 2.5975 mmol, 5.0 eq). The reaction mixture heated to 70° C. and stirred at that temperature for 2 h. After completion of reaction the mixture was poured into ice-cold water and the precipitate was filtered, washed with water and hexanes to obtain 0.165 g of methyl 2-(4′-fluoro-N-methyl-[1,1′-biphenyl]-3-carboxamido)-5-oxo-5H-thieno[3,2-b]pyran-6-carboxylate (7); Yield: 72.60%; MS (ES): m/z 438.44 [M+H]⁺; ¹H NMR (DMSO-d6, 400 MHZ): 8.80 (s, 1H), 7.99 (s, 2H), 7.78 (s, 1H), 7.74-7.70 (m, 2H), 7.48-7.46 (d, J=8 Hz 2H), 7.09-6.98 (m, 2H), 3.69 (s, 3H), 3.25 (s, 3H).

### Step 8

A three-necked RBF was charged with Intermediate (7) (0.165 g, 0.3771 mmol, 1.0 eq) in dichloromethane (20 mL) and trimethylsilyl iodide (0.3 mL, 1.8855 mmol, 5 eq) at room temperature. The reaction mixture was stirred for 16 h at that temperature. After completion of reaction the mixture was concentrated under reduced pressure to obtain the crude material which was triturated with methanol to obtain 0.120 g of 2-(4′-fluoro-N-methyl-[1,1′-biphenyl]-3-carboxamido)-5-oxo-5H-thieno[3,2-b]pyran-6-carboxylic acid (Example 31); Yield: 75.14%; MS (ES): m/z 424.41[M+H]⁺; ¹H NMR (DMSO-d6, 400 MHZ): 12.68 (s, 1H), 8.91 (s, 1H), 7.95 (s, 1H), 7.89 (s, 1H), 7.810-7.796 (m, 2H), 7.65-7.64 (d, J=5 Hz 2H), 7.33-7.31, (t, J=16 Hz, 2H), 7.17 (s, 1H), 3.55 (s, 3H).

### Example 32

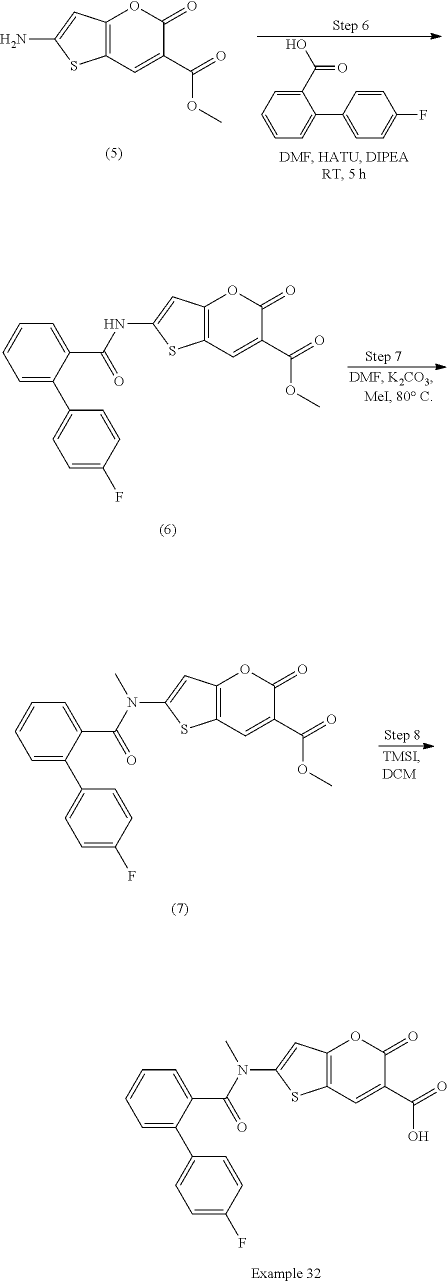

### Step 6

A three-necked RBF was charged with 4′-fluoro-[1,1′-biphenyl]-2-carboxylic acid (0.3 g, 1.3875 mmol, 1.0 eq) in DMF (10 mL) and cooled to 0° C. HATU (0.790 g, 2.0812 mmol, 1.5 eq) was added and the mixture was stirred 15 min. Intermediate (5) (0.250 g, 1.11 mmol, 0.8 eq) and DIPEA (1.0 mL, 5.55 mmol, 4.0 eq) were added and the mixture stirred at room temperature for 0.5 h. After completion of reaction the mixture was transferred into water and extracted with ethyl acetate. Organic layers were combined, washed with brine, dried over anhydrous sodium sulphate and concentrated under reduced pressure to obtain the crude material. The crude was purified by silica gel column chromatography (2.8% methanol in dichloromethane) to obtain 0.165 g of methyl 2-(4′-fluoro-[1,1′-biphenyl]-2-carboxamido)-5-oxo-5H-thieno[3,2-b]pyran-6-carboxylate (6); Yield: 35.11%; MS (ES): m/z 424.21[M+H]⁺; ¹H NMR (DMSO-d6, 400 MHZ): 11.19 (s, 1H), 8.51 (s, 1H), 8.00 (s, 2H), 7.75 (s, 1H), 7.62-7.52 (m, 2H), 7.44-7.42 (d, J=8 Hz 2H), 7.10-7.08 (d, J=8 Hz, 2H), 3.61 (s, 3H)

### Step 7

A three-necked RBF was charged with Intermediate (6) (0.165, 0.3898 mmol, 1.0 eq) in N,N-dimethylformamide (10 mL) and potassium carbonate (0.135 g, 0.9745 mmol, 2.5 eq) and cooled to ° C. Methyl iodide (0.275 g, 1.949 mmol, 5.0 eq) was added dropwise. After the addition was complete the mixture was heated to 70° C. for 2 h. The mixture was poured into ice-cold water and the precipitate was filtered and washed with water and hexanes to obtain 0.105 g of methyl 2-(4′-fluoro-N-methyl-[1,1′-biphenyl]-2-carboxamido)-5-oxo-5H-thieno[3,2-b]pyran-6-carboxylate (7); Yield: 61.60%; MS (ES): m/z 438.44[M+H]⁺; ¹H NMR (DMSO-d6, 400 MHZ): 8.80 (s, 1H), 8.05 (s, 2H), 7.82 (s, 1H), 7.79-7.72 (m, 2H), 7.42-7.40 (d, J=8 Hz 2H), 7.06-6.95 (m, 2H), 3.63 (s, 3H), 3.20 (s, 3H).

### Step 8

A three-necked RBF was charged with Intermediate (7) (0.105 g, 0.24 mmol, 1.0 eq) in dichloromethane (20 mL) and trimethylsilyl iodide (0.1 mL, 0.6 mmol, 2.5 eq) at room temperature. The reaction mixture was stirred for 16 h at that temperature. After completion of reaction the mixture was concentrated under reduced pressure to obtain the crude material which was triturated with methanol to obtain 0.82 g of 2-(4′-fluoro-N-methyl-[1,1′-biphenyl]-2-carboxamido)-5-oxo-5H-thieno[3,2-b]pyran-6-carboxylic acid (Example 32); Yield: 80.68%; MS (ES): m/z 424.41 [M+H]⁺; ¹H NMR (DMSO-d6, 400 MHZ): 12.679 (s, 1H), 8.870 (s, 1H), 7.675-7.56 (m, 4H), 7.381 (s, 2H), 7.252, 7.231, 7.211 (t, J=16 Hz, 2H), 6.964 (s, 1H), 3.105 (s, 3H).

### Example 33

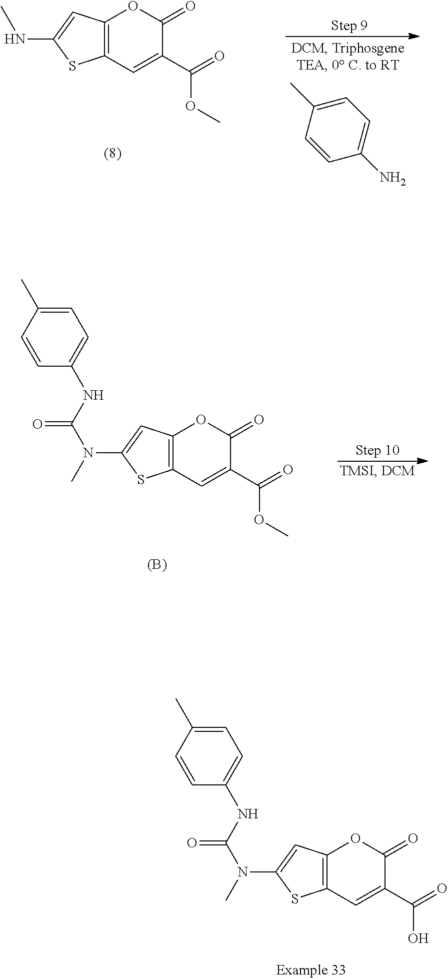

### Step 9

To a stirred solution of p-toluidine (0.380 g, 3.546 mmol, 1.0 eq) in dichloromethane (15 mL) was added triphosgene (0.368 g, 1.241 mmol, 0.35 eq) at 0° C. After 15 min Intermediate 8 (0.254 g, 1.063 mmol, 0.30 eq) was added followed by triethylamine (1.5 mL, 10.63 mmol, 3.0 eq) and the mixture was stirred it at room temperature for 2 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(1-methyl-3-(p-tolyl)ureido)-5-oxo-5H-thieno[3,2-b]pyran-6-carboxylate (B). (0.190 g Yield: 48.06%). MS (ES):373.20 m/z [M+H]⁺.

### Step 10

To a solution of Intermediate-(B) (0.190 g, 0.510 mmol, 1.0 eq) in dichloromethane (20 mL) was added trimethylsilyl iodide (0.18 mL, 1.275 mmol, 2.5 eq) at room temperature and reaction mixture was stirred for 16 h at room temperature. After completion of reaction, the mixture was concentrated under reduced pressure to obtain crude material which was triturated with methanol to obtain 2-(1-methyl-3-(p-tolyl)ureido)-5-oxo-5H-thieno[3,2-b]pyran-6-carboxylic acid (Example 33). (0.116 g, Yield: 63.44%), MS (ES): 359.0 m/z [M+H]⁺, LCMS purity: 97.67%, HPLC purity: 97.62%, 1H NMR (DMSO-d6, 400 MHZ): 12.51 (s, 1H), 9.38 (s, 1H), 8.80 (s, 1H), 7.42-7.40 (d, J=8 Hz, 2H), 7.18-7.16 (d, J=8 Hz, 2H), 6.94 (s, 1H), 3.61 (s, 3H), 2.30 (s, 3H).

### Example 34

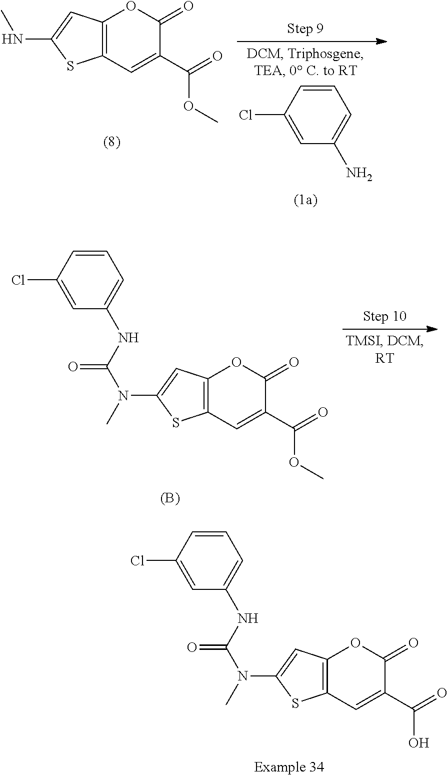

### Step 9

To a stirred solution of 3-chloroaniline (1a) (0.430 g, 3.385 mmol, 1.0 eq) in dichloromethane (15 mL) was added triphosgene (0.350 g, 1.179 mmol, 0.35 eq) at 0° C. After 15 min Intermediate 8 (0.242 g, 1.011 mmol, 0.30 eq) was added followed by triethylamine (1.42 mL, 10.11 mmol, 3.0 eq) and the mixture was stirred it at room temperature for 2 h. After completion of reaction, reaction mixture was poured in to water (50 mL) and extracted with dichloromethane (2×50 mL). Organic layers were combined, washed with brine (2×25 mL), dried over sodium sulphate and concentrated under reduced pressure to obtain crude material. The crude material was purified by column chromatography using silica gel and the desired product was eluted at 1.8% MeOH in DCM afford methyl 2-(3-(3-chlorophenyl)-1-methylureido)-5-oxo-5H-thieno[3,2-b]pyran-6-carboxylate (B) (0.170 g Yield: 42.79%). MS (ES):393.0 m/z [M+H]⁺.

### Step 10

In 50 mL, 2-necked RBF, solution of Intermediate-(B) (0.170 g, 0.432 mmol, 1.0 eq) in dichloromethane (20 mL) was added trimethylsilyl iodide (0.21 mL, 1.081 mmol, 2.5 eq) at room temperature and reaction mixture was stirred for 16 h at room temperature. After completion of reaction the mixture was concentrated under reduced pressure to obtain crude material which was triturated with methanol to obtain pure 2-(3-(3-chlorophenyl)-1-methylureido)-5-oxo-5H-thieno[3,2-b]pyran-6-carboxylic acid (Example 34). (0.103 g, Yield: 62.83%), MS (ES): 379.4 m/z [M+H]⁺, ¹H NMR (DMSO-d6, 400 MHZ): 12.55 (s, 1H), 9.59 (s, 1H), 8.81 (s, 1H), 7.68 (s, 1H), 7.52-7.50 (d, J=8 Hz, 1H), 7.40-6.96 (m, 3H), 3.60 (s, 3H).

### Example 35

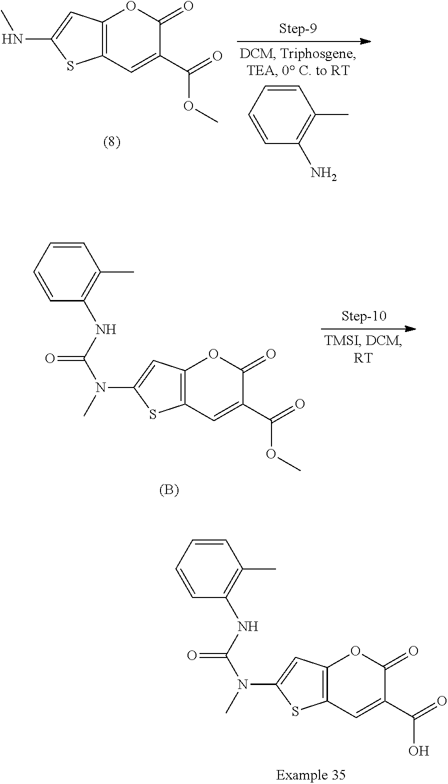

### Step 9

To a stirred solution of o-toluidine (0.190 g, 1.77 mmol, 1.0 eq) in dichloromethane (10 mL) was added triphosgene (0.184 g, 0.620 mmol, 0.35 eq) at 0° C. After 15 min Intermediate 8 (0.127 g, 0.531 mmol, 0.3 eq) was added followed by triethylamine (0.7 mL, 5.31 mmol, 3.0 eq) and the mixture was stirred it at room temperature for 2 h. After completion of reaction the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford methyl 2-(1-methyl-3-(o-tolyl)ureido)-5-oxo-5H-thieno[3,2-b]pyran-6-carboxylate (B) (0.080 g Yield: 40.47%). MS (ES):373.05 m/z [M+H]⁺.

### Step 10

To a solution of Intermediate (B) (0.080 g, 0.215 mmol, 1.0 eq) in dichloromethane (20 mL) was added trimethylsilyl iodide (0.08 mL, 0.537 mmol, 2.5 eq) at room temperature and reaction mixture was stirred for 16 h at room temperature. After completion of reaction the mixture was concentrated under reduced pressure to obtain crude material which was triturated with methanol to obtain 2-(1-methyl-3-(o-tolyl)ureido)-5-oxo-5H-thieno[3,2-b]pyran-6-carboxylic acid (Example 35). (0.035 g, Yield: 45.46%), MS (ES): 359.05 m/z [M+H]⁺, ¹H NMR (DMSO-d6, 400 MHZ): 12.50 (s, 1H), 9.20 (s, 1H), 8.79 (s, 1H), 7.28-7.22 (m, 4H), 6.94 (s, 1H), 3.63 (s, 3H), 2.23 (s, 3H).

### Example 36

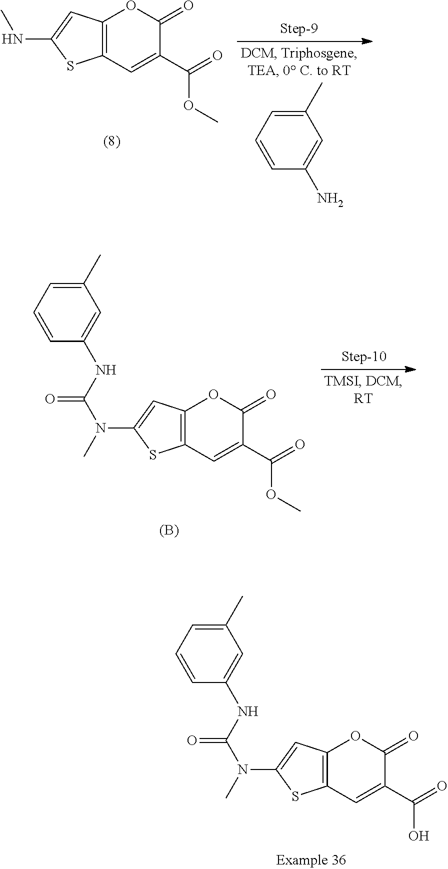

### Step 9

To a stirred solution of m-toluidine (0.190 g, 1.77 mmol, 1.0 eq) in dichloromethane (10 mL) was added triphosgene (0.184 g, 0.620 mmol, 0.35 eq) at 0° C. After 15 min Intermediate 8 (0.127 g, 0.531 mmol, 0.3 eq) was added followed by triethylamine (0.7 mL, 5.31 mmol, 3.0 eq) and the mixture was stirred it at room temperature for 2 h. After completion of reaction the mixture was poured in to water (50 mL) and extracted with dichloromethane (2×50 mL). Organic layers were combined, washed with brine (2×25) dried over sodium sulphate and concentrated under reduced pressure. The crude material was purified by column chromatography using silica gel and the desired product was eluted at 1.8% MeOH in DCM afford pure methyl 2-(1-methyl-3-(m-tolyl)ureido)-5-oxo-5H-thieno[3,2-b]pyran-6-carboxylate (B) (0.075 g Yield: 37.94%). MS (ES):373.04 m/z [M+H]⁺.

### Step 10

To a solution of Intermediate (B) (0.075 g, 0.201 mmol, 1.0 eq) in dichloromethane (20 mL) was added trimethylsilyl iodide (0.08 mL, 0.503 mmol, 2.5 eq) at room temperature and reaction mixture was stirred for 16 h at room temperature. After completion of reaction the mixture was concentrated under reduced pressure to obtain crude material which was triturated with methanol to obtain pure 2-(1-methyl-3-(m-tolyl)ureido)-5-oxo-5H-thieno[3,2-b]pyran-6-carboxylic acid (Example 36). (0.025 g, Yield: 34.64%), MS (ES): 359.2 m/z [M+H]⁺, ¹H NMR (DMSO-d6, 400 MHZ): 12.52 (s, 1H), 9.39 (s, 1H), 8.80 (s, 1H), 7.36-7.21 (m, 3H), 6.95-6.94 (d, 2H), 3.60 (s, 3H), 2.31 (s, 3H).

### Example 37

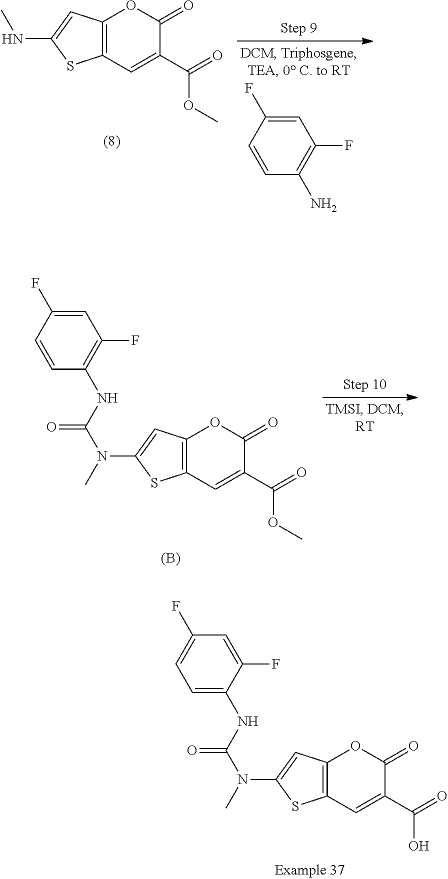

### Step 9

To a stirred solution of 2,4-difluoroaniline (1a) (0.220 g, 1.70 mmol, 1.0 eq) in dichloromethane (10 mL) was added triphosgene (0.177 g, 0.596 mmol, 0.35 eq) at 0° C. After 15 min Intermediate 8 (0.122 g, 0.511 mmol, 0.3 eq) was added followed by triethylamine (0.7 mL, 5.11 mmol, 3.0 eq). The mixture was stirred at room temperature for 2 h. After completion of reaction, reaction mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford methyl 2-(3-(2,4-difluorophenyl)-1-methylureido)-5-oxo-5H-thieno[3,2-b]pyran-6-carboxylate (B) (0.056 g Yield: 27.85%). MS (ES):395.05 m/z [M+H]⁺.

### Step 10

To a solution of Intermediate (B) (0.056 g, 0.142 mmol, 1.0 eq) in dichloromethane (20 mL) was added trimethylsilyl iodide (0.05 mL, 0.355 mmol, 2.5 eq) at room temperature and the mixture was stirred for 16 h at room temperature. After completion of reaction, the mixture was concentrated under reduced pressure to obtain crude material which was triturated with methanol to obtain pure 2-(3-(2,4-difluorophenyl)-1-methylureido)-5-oxo-5H-thieno[3,2-b]pyran-6-carboxylic acid (Example 37). (0.015 g, Yield: 27.77%), MS (ES): 381.0 m/z [M+H]⁺, H NMR (DMSO-d6, 400 MHZ): 12.52 (s, 1H), 9.43 (s, 1H), 8.79 (s, 1H), 7.49-7.35 (m, 2H), 7.16-7.11 (t, 1H), 6.95 (s, 1H), 3.60 (s, 3H).

### Example 38

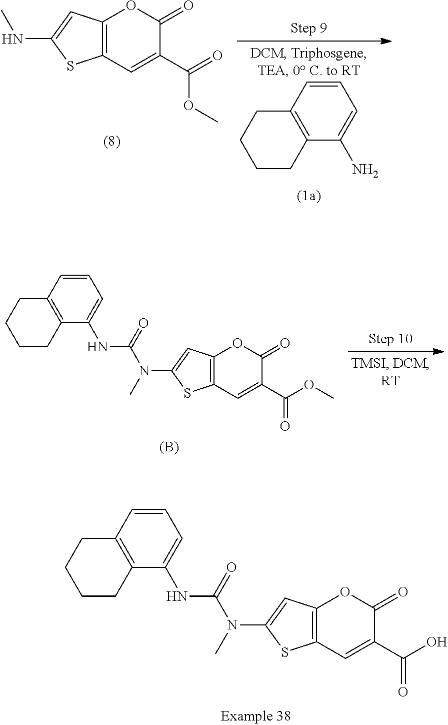

### Step 9

To a stirred solution of 5,6,7,8-tetrahydronaphthalen-1-amine (1a) (0.210 g, 1.427 mmol, 1.0 eq) in dichloromethane (10 mL) was added triphosgene (0.148 g, 0.499 mmol, 0.35 eq) at 0° C. After 15 Intermediate 8 (0.102 g, 0.428 mmol, 0.3 eq) was added followed by triethylamine (0.6 mL, 4.282 mmol, 3.0 eq). The mixture was stirred it at room temperature for 2 h. After completion of reaction the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford methyl 2-(1-methyl-3-(5,6,7,8-tetrahydronaphthalen-1-yl)ureido)-5-oxo-5H-thieno[3,2-b]pyran-6-carboxylate (B) (0.095 g Yield: 54.02%). MS (ES):413.1 m/z [M+H]⁺.

### Step 10

To a solution of Intermediate-B (0.095 g, 0.230 mmol, 1.0 eq) in dichloromethane (20 mL) was added trimethylsilyl iodide (0.08 mL, 0.576 mmol, 2.5 eq) at room temperature and reaction mixture was stirred for 16 h at room temperature. After completion of reaction the mixture was concentrated under reduced pressure to obtain crude material which was triturated with methanol to obtain 2-(1-methyl-3-(5,6,7,8-tetrahydronaphthalen-1-yl)ureido)-5-oxo-5H-thieno[3,2-b]pyran-6-carboxylic acid (Example 38). (0.030 g, Yield: 32.69%), MS (ES): 399.2 m/z [M+H]⁺, ¹H NMR (DMSO-d6, 400 MHZ): 12.50 (s, 1H), 9.08 (s, 1H), 8.78 (s, 1H), 7.14-7.02 (m, 3H), 6.93 (s, 1H), 3.62 (s, 3H), 2.78 (s, 2H), 2.62 (s, 2H), 1.73 (s, 4H).

### Example 39

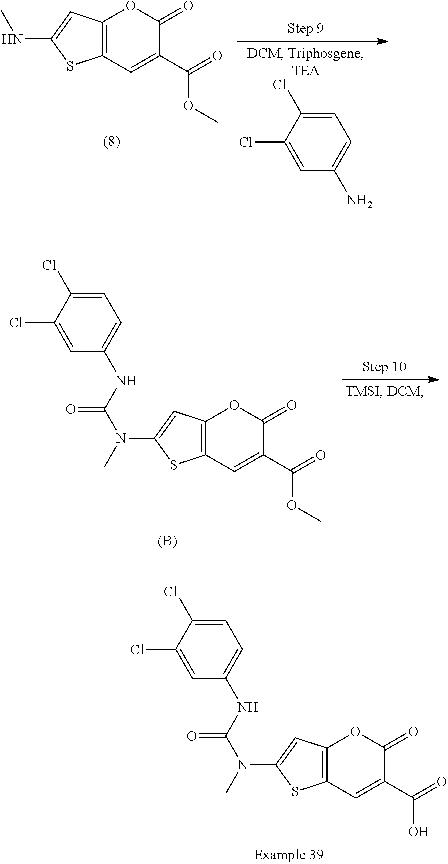

### Step 9

To a stirred solution of 3,4-dichloroaniline (0.270 g, 1.67 mmol, 1.0 eq) in dichloromethane (10 mL) was added triphosgene (0.174 g, 0.58 mmol, 0.35 eq) at 0° C. After 15 min Intermediate 8 (0.120 g, 0.503 mmol, 0.3 eq) was added followed by triethylamine (0.7 mL, 5.03 mmol, 3.0 eq) and the mixture was stirred at room temperature for 2 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(3-(3,4-dichlorophenyl)-1-methylureido)-5-oxo-5H-thieno[3,2-b]pyran-6-carboxylate (B) (0.074 g Yield: 34.53%). MS (ES):427.0 m/z [M+H]⁺.

### Step 10

To solution of Intermediate (B) (0.074 g, 0.173 mmol, 1.0 eq) in dichloromethane (20 mL) was added trimethylsilyl iodide (0.06 mL, 0.433 mmol, 2.5 eq) at room temperature. The mixture was stirred for 16 h. After completion of reaction the mixture was concentrated under reduced pressure to obtain crude material which was triturated with methanol to obtain pure 2-(3-(3,4-dichlorophenyl)-1-methylureido)-5-oxo-5H-thieno[3,2-b]pyran-6-carboxylic acid (Example 39). (0.024 g, Yield: 33.53%), MS (ES): 413.01 m/z [M+H]⁺, ¹H NMR (DMSO-d6, 400 MHZ): 12.55 (s, 1H), 9.67 (s, 1H), 8.81 (s, 1H), 7.87 (s, 1H), 7.63-7.54 (m, 2H), 6.97 (s, 1H), 3.60 (s, 3H).

### Example 40

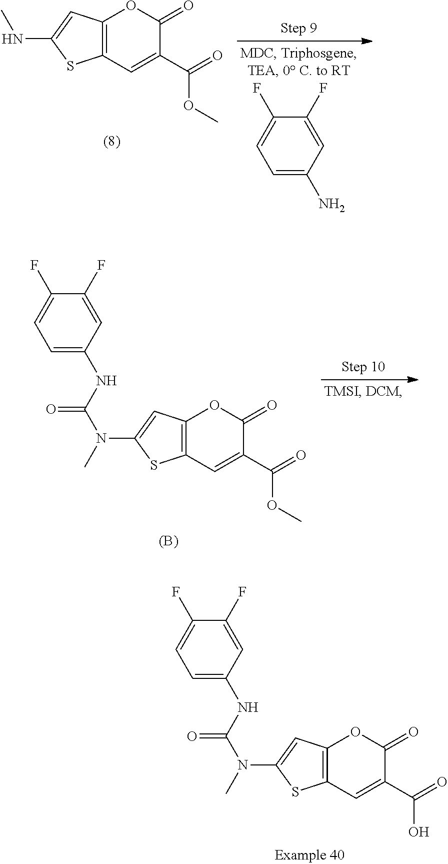

### Step 9

To a stirred solution of 3,4-difluoroaniline (0.220 g, 1.703 mmol, 1.0 eq) in dichloromethane (10 mL) was added triphosgene (0.177 g, 0.596 mmol, 0.35 eq) at 0° C. After 15 min Intermediate 8 (0.122 g, 0.511 mmol, 0.3 eq) was added followed by triethylamine (0.7 mL, 5.111 mmol, 3.0 eq) and the mixture was stirred at room temperature for 2 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(3-(3,4-difluorophenyl)-1-methylureido)-5-oxo-5H-thieno[3,2-b]pyran-6-carboxylate (B) (0.060 g Yield: 29.84%). MS (ES):395.05 m/z [M+H]⁺.

### Step 10

To a solution of Intermediate (B) (0.060 g, 0.152 mmol, 1.0 eq) in dichloromethane (20 mL) was added trimethylsilyl iodide (0.05 mL, 0.380 mmol, 2.5 eq) at room temperature and reaction mixture was stirred for 16 h at room temperature. After completion of reaction, the mixture was concentrated under reduced pressure to obtain crude material which was triturated with methanol to obtain pure 2-(3-(3,4-difluorophenyl)-1-methylureido)-5-oxo-5H-thieno[3,2-b]pyran-6-carboxylic acid (Example 40). (0.008 g, Yield: 13.83%), MS (ES): 381.20 m/z [M+H]⁺, ¹H NMR (DMSO-d6, 400 MHZ): 12.53 (s, 1H), 9.63 (s, 1H), 8.82 (s, 1H), 7.68-7.63 (m, 1H), 7.48-7.37 (m, 2H), 6.84 (s, 1H), 3.61 (s, 3H).

### Example 41

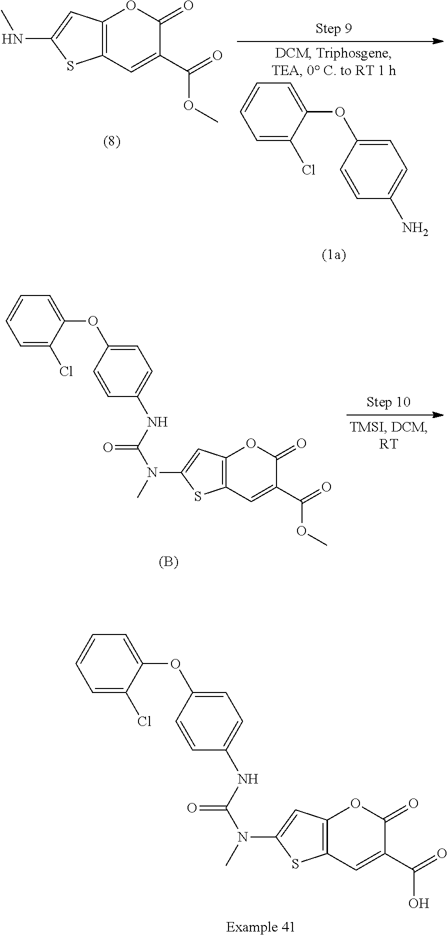

### Step 9

To a stirred solution of 4-(2-chlorophenoxy)aniline (1a) (0.36 g, 1.67 mmol, 1.0 eq) in dichloromethane (10 mL) was added triphosgene (0.17 g, 0.58 mmol, 0.35 eq) at 0° C. After 15 min Intermediate (8) (0.12 g, 0.503 mmol, 0.3 eq) was added followed by triethylamine (0.7 mL, 5.03 mmol, 3.0 eq) and the mixture was stirred at room temperature for 2 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified through column chromatography using silica gel and the desired product was eluted at 1.8% MeOH in DCM afford pure methyl 2-(3-(4-(2-chlorophenoxy)phenyl)-1-methylureido)-5-oxo-5H-thieno[3,2-b]pyran-6-carboxylate (B) (0.070 g Yield: 28.78%). MS (ES): 485.9 m/z [M+H]⁺.

### Step 10

In 50 mL, 2-necked RBF, solution of Intermediate-(B) (0.070 g, 0.14 mmol, 1.0 eq) in dichloromethane (20 mL) was added trimethylsilyl iodide (0.051 mL, 0.36 mmol, 2.5 eq) at room temperature and the mixture was stirred for 16 h at room temperature. After completion of reaction, the mixture was concentrated under reduced pressure to obtain crude material which was triturated with methanol to obtain pure 2-(3-(4-(2-chlorophenoxy)phenyl)-1-methylureido)-5-oxo-5H-thieno[3,2-b]pyran-6-carboxylic acid (Example 41). (0.027 g, Yield: 39.72%), MS (ES): 471.8 m/z [M+H]⁺, ¹H NMR (DMSO-d6, 400 MHZ): 12.52 (s, 1H), 9.50 (s, 1H), 8.81 (s, 1H), 7.63-7.62 (m, 1H), 7.61-7.60 (m, 2H), 7.55-7.36 (m, 1H), 7.25-7.21 (m, 1H), 7.10-7.07 (m, 1H) 7.00-6.95 (m, 3H), 3.36 (s, 3H).

### Example 42

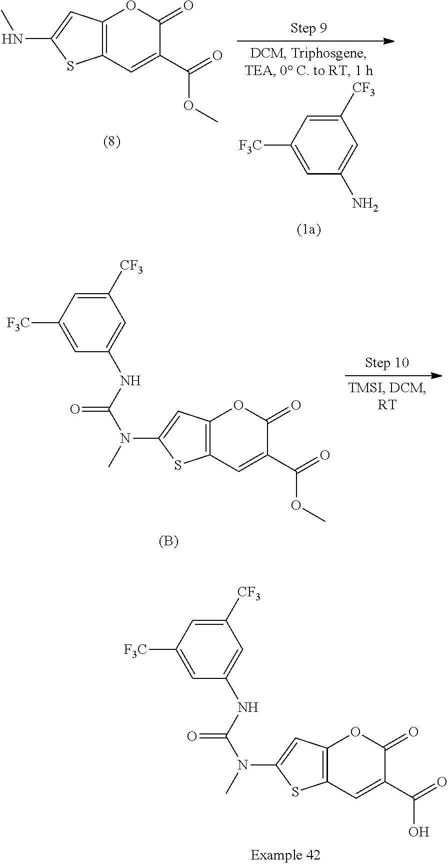

### Step 9

To a stirred solution of 3,5-bis(trifluoromethyl)aniline (1a) (0.380 g, 1.68 mmol, 1.0 eq) in dichloromethane (10 mL) was added triphosgene (0.17 g, 0.58 mmol, 0.35 eq) at 0° C. After 15 min Intermediate (8) (0.120 g, 0.503 mmol, 0.3 eq) was added followed by triethylamine (0.7 mL, 5.03 mmol, 3.0 eq). The mixture was stirred at room temperature for 2 h. After completion of reaction The mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford methyl 2-(3-(3,5-bis(trifluoromethyl)phenyl)-1-methylureido)-5-oxo-5H-thieno[3,2-b]pyran-6-carboxylate (B) (0.065 g Yield: 26.21%). MS (ES): 495.3 m/z [M+H]⁺.

### Step 10

To a solution of Intermediate (B) (0.065 g, 0.13 mmol, 1.0 eq) in dichloromethane (20 mL) was added trimethylsilyl iodide (0.050 mL, 0.32 mmol, 2.5 eq) at room temperature. The mixture was stirred for 16 h at room temperature. After completion of reaction the mixture was concentrated under reduced pressure to obtain crude material which was triturated with methanol to obtain 2-(3-(3,5-bis(trifluoromethyl)phenyl)-1-methylureido)-5-oxo-5H-thieno[3,2-b]pyran-6-carboxylic acid (Example 42). (0.022 g, Yield: 34.83%), MS (ES): 481.3 m/z [M+H]⁺, ¹H NMR (DMSO-d6, 400 MHZ): 12.59 (s, 1H), 10.01 (s, 1H), 8.83 (s, 1H), 8.30 (s, 2H), 7.84 (s, 1H), 7.03 (s, 1H), 3.34 (s, 3H).

### Example 43

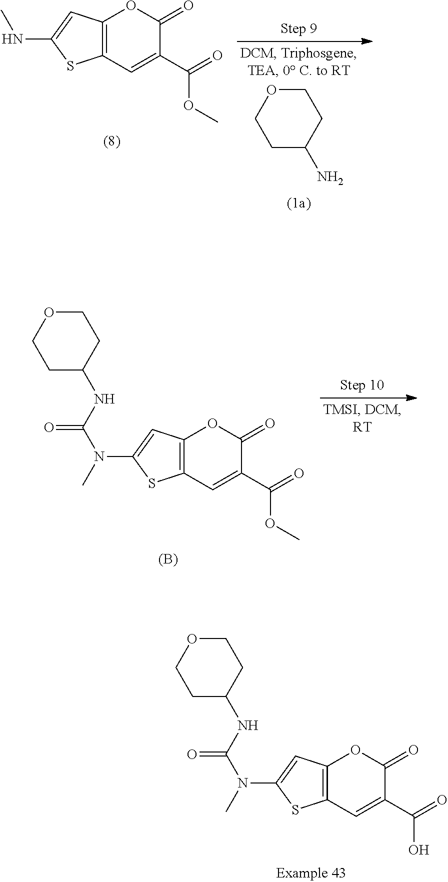

### Step 9

To a stirred solution of tetrahydro-2H-pyran-4-amine (1a) (0.17 g, 1.68 mmol, 1.0 eq) in dichloromethane (10 mL) was added triphosgene (0.174 g, 0.58 mmol, 0.35 eq) at 0° C. After 15 min Intermediate (8) (0.120 g, 0.50 mmol, 0.3 eq) was added followed by triethylamine (0.7 mL, 5.03 mmol, 3.0 eq). The mixture was stirred at room temperature for 2 h. After completion of reaction the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford methyl 2-(1-methyl-3-(tetrahydro-2H-pyran-4-yl)ureido)-5-oxo-5H-thieno[3,2-b]pyran-6-carboxylate (9) (0.065 g Yield: 35.37%). MS (ES): 367.3 m/z [M+H]⁺.

### Step 10

To a solution of Intermediate-(B) (0.065 g, 0.17 mmol, 1.0 eq) in dichloromethane (20 mL) was added trimethylsilyl iodide (0.063 mL, 0.44 mmol, 2.5 eq) at room temperature and the mixture was stirred for 16 h at room temperature. After completion of reaction the mixture was concentrated under reduced pressure to obtain crude material which was triturated with methanol to obtain 2-(3-(3,5-bis(trifluoromethyl)phenyl)-1-methylureido)-5-oxo-5H-thieno[3,2-b]pyran-6-carboxylic acid (Example 43). (0.015 g, Yield: 24.0%), MS (ES): 353.36 m/z [M+H]⁺, ¹H NMR (DMSO-d6, 400 MHZ): 12.47 (s, 1H), 8.77 (s, 1H), 7.48-7.48 (d, 1H), 6.83 (s, 1H), 3.90-3.80 (m, 2H), 3.79-3.76 (m, 1H), 3.36 (s, 3H), 3.34-3.24 (m, 2H), 1.78-1.75 (m, 2H), 1.63-1.53 (m, 2H).

### Example 44

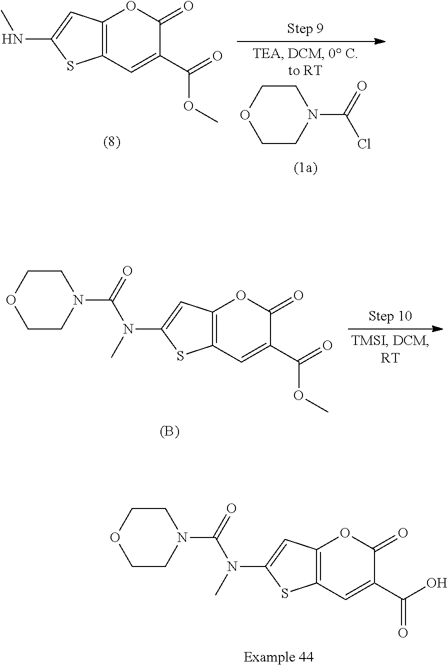

### Step 9

To a stirred solution of Intermediate 8 (0.100 g, 0.417 mmol, 1.0 eq) in dichloromethane (10 mL) was added DMAP (0.005 g, 0.041 mmol, 0.1 eq) and triethylamine (0.17 mL, 1.253 mmol, 3.0 eq) at 0° C. After 10 min morpholine-4-carbonyl chloride (1a) (0.094 g, 0.626 mmol, 1.5 eq) was added and resulting mixture was stirred at room temperature for 12 h. After completion of reaction the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(N-methylmorpholine-4-carboxamido)-5-oxo-5H-thieno[3,2-b]pyran-6-carboxylate (B) (0.107 g Yield: 72.65%). MS (ES): 353.08 m/z [M+H]⁺.

### Step 10

To a solution of Intermediate (B) (0.107 g, 0.303 mmol, 1.0 eq) in dichloromethane (20 mL) was added trimethylsilyl iodide (0.1 mL, 0.759 mmol, 2.5 eq) at room temperature and reaction mixture was stirred for 16 h at that temperature. After completion of reaction, the mixture was concentrated under reduced pressure to obtain crude material which was triturated with methanol to obtain pure 2-(N-methylmorpholine-4-carboxamido)-5-oxo-5H-thieno[3,2-b]pyran-6-carboxylic acid (Example 44). (0.060 g, Yield: 58.40%), MS (ES): 339.2 m/z [M+H]⁺, ¹H NMR (DMSO-d6, 400 MHZ): 12.51 (s, 1H), 8.78 (s, 1H), 6.80 (s, 1H), 3.64-3.63 (t, 4H), 3.41 (s, 3H), 3.37-3.36 (t, 4H).

### Example 45

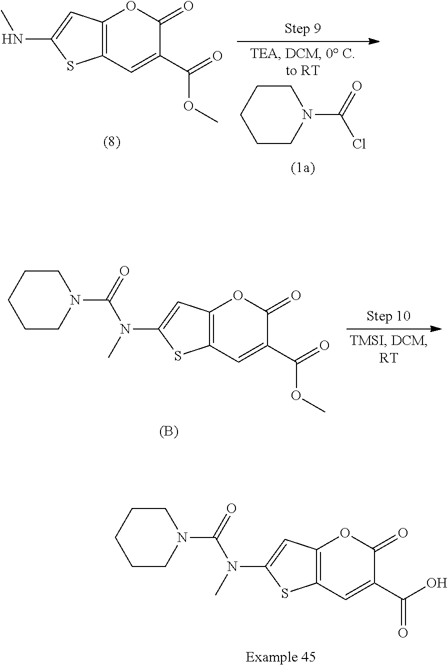

### Step 9

To a stirred solution of Intermediate 8 (0.100 g, 0.417 mmol, 1.0 eq) in dichloromethane (10 mL) was added DMAP (0.005 g, 0.041 mmol, 0.1 eq) and triethylamine (0.17 mL, 1.253 mmol, 3.0 eq) at 0° C. After 10 min Piperidine-1-carbonyl chloride (1a) (0.092 g, 0.626 mmol, 1.5 eq) was added and the resulting mixture was stirred at room temperature for 12 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(N-methylpiperidine-1-carboxamido)-5-oxo-5H-thieno[3,2-b]pyran-6-carboxylate (B) (0.110 g Yield: 75.11%). MS (ES): 351.10 m/z [M+H]⁺.

### Step 10

To a solution of Intermediate (B) (0.110 g, 0.314 mmol, 1.0 eq) in dichloromethane (20 mL) was added trimethylsilyl iodide (0.1 mL, 0.785 mmol, 2.5 eq) at room temperature and reaction mixture was stirred for 16 h at room temperature. After completion of reaction the mixture was concentrated under reduced pressure to obtain crude material which was triturated with methanol to obtain 2-(N-methylpiperidine-1-carboxamido)-5-oxo-5H-thieno[3,2-b]pyran-6-carboxylic acid (Example 45). (0.036 g, Yield: 34.09%), MS (ES): 337.2 m/z [M+H]⁺, ¹H NMR (DMSO-d6, 400 MHZ): 12.49 (s, 1H), 8.77 (s, 1H), 6.75 (s, 1H), 3.39 (s, 4H), 3.31-3.30 (t, 3H), 1.57 (s, 6H).

### Example 46

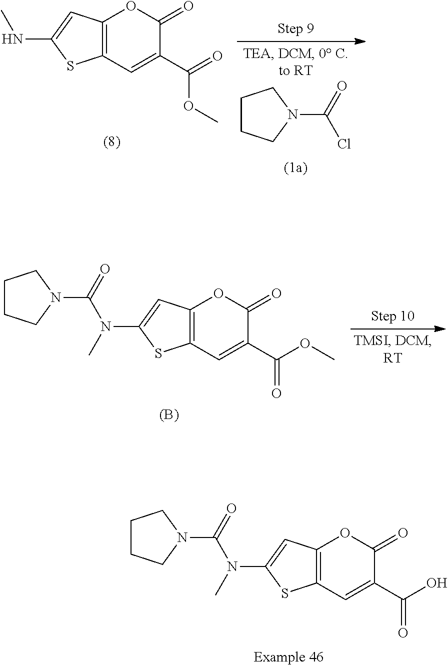

### Step 9

To a stirred solution of Intermediate (8)(0.120 g, 0.50 mmol, 1.0 eq) in dichloromethane (10 mL) was added DMAP (0.006 g, 0.050 mmol, 0.1 eq) and trimethylamine (0.20 mL, 1.50 mmol, 3.0 eq) at 0° C. After 10 min pyrrolidine-1-carbonyl chloride (1a) (0.10 g, 0.75 mmol, 1.5 eq) was added to the reaction mixture. The mixture was stirred at room temperature for 12 h. After completion of reaction, the mixture was poured in to water (50 mL) and extracted with dichloromethane (2×50 mL). Organic layers were combined, washed with brine (2×25 mL), dried over sodium sulphate and concentrated under reduced pressure. The crude material was purified by column chromatography using silica gel and the desired product was eluted at 1.8% MeOH in DCM afford pure methyl 2-(N-methylpyrrolidine-1-carboxamido)-5-oxo-5H-thieno[3,2-b]pyran-6-carboxylate (B) (0.023 g Yield: 13.63%). MS (ES): 337.35 m/z [M+H]⁺.

### Step 10

To a solution of Intermediate-(B) (0.023 g, 0.068 mmol, 1.0 eq) in dichloromethane (10 mL) was added trimethylsilyl iodide (0.025 mL, 0.171 mmol, 2.5 eq) at room temperature and the mixture was stirred for 16 h at room temperature. After completion of reaction the mixture was concentrated under reduced pressure to obtain crude material which was triturated with methanol to obtain 2-(N-methylpyrrolidine-1-carboxamido)-5-oxo-5H-thieno[3,2-b]pyran-6-carboxylic acid (Example 46). (0.008 g, Yield: 36.30%), ¹H NMR (DMSO-d6, 400 MHZ): 12.47 (s, 1H), 8.78 (s, 1H), 6.78 (s, 1H), 3.43-3.25 (m, 7H), 1.85-1.75 (m, 4H).

### Example 47: MTS Cell Proliferation Assay

Cytotoxicity of the inhibition of monocarboxylate transporters of the invention was determined and shown in Table 1. The anti-proliferation effect of MCT inhibition was investigated across a panel of solid and haematological tumor cell lines. Cells were routinely cultured in their appropriate growth medium. On day 1, between 10,000-25,000 cells/well (e.g., Hs578t: 15,000 cells/well, SiHa: 10,000 cells/well, and MDA-MB-231: 25,000 cells/well) were plated into 96-well plates. 100 μL of phosphate buffered saline solution was added to the external wells to prevent media evaporation. Plates were incubated in growth medium overnight at 37° C. in the presence of 5% CO₂. On day 2, dry weight compound stocks were dissolved to a concentration of 10 mM in 100% DMSO. Compounds were further diluted in the assay medium; 10 mM lactate medium (without glucose, pyruvate, and glutamine) or RPMI 1640 medium (without pyruvate and glutamine) to generate a final dose range of 1 nM to 10 μM. Growth medium in the 96-well plate was replaced with the assay medium (10 mM lactate medium or RPMI medium or appropriate medium), and compounds were added to each well in the plate at different concentrations via serial dilution or pre-prepared solutions in assay medium. Plates were then incubated at 37° C. in the presence of 5% CO₂ for a further 72-96 hours. On day 2-5, assay media was changed to 100 uL of DMEM/F12 and 20 μL of CellTiter 96 AQ MTS reagent was added to each well and the plate was returned to the incubator for 1-2 hours. MTS is bioreduced by NADPH or NADH produced by dehydrogenase enzymes in metabolically active cells into a coloured formazan product that is soluble in tissue culture medium. The amount of coloured formazan product is directly proportional to the number of living cells in culture. The absorbance of the plates was read on a Spectramax M5e plate reader using 490 nM measurement wavelength. Dose response curves were plotted and IC₅₀ values were calculated using GraphPad Prism. The IC₅₀ value is equivalent to the concentration of compound that causes 50% inhibition of growth calculated from the compound treated signal to the vehicle treated signal.

Cytotoxicity of selected compounds is listed in Table 1, where IC₅₀: A=<1 uM; B=1-10 uM; C=>10 uM; and NT=Not Tested.

TABLE 1
Compounds and Assay Results

| | | | Cytotoxicity |
|---|---|---|---|
| Example | Structure | Name | [IC₅₀: μM] |
| 1 | 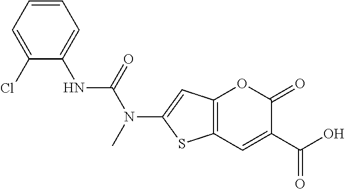 [SMILES: CN(C(=O)Nc1ccccc1Cl)c1cc2oc(=O)c(C(=O)O)cc2s1] | 2-(3-(2-chlorophenyl)-1- methylureido)-5-oxo-5H- thieno[3,2-b]pyran-6- carboxylic acid | A |
| 2 | 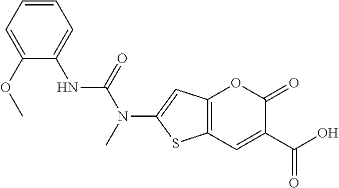 [SMILES: COc1ccccc1NC(=O)N(C)c1cc2oc(=O)c(C(=O)O)cc2s1] | 2-(3-(2-methoxyphenyl)-1- methylureido)-5-oxo-5H- thieno[3,2-b]pyran-6- carboxylic acid | A |
| 3 | 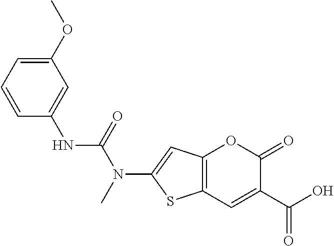 [SMILES: COc1cccc(NC(=O)N(C)c2cc3oc(=O)c(C(=O)O)cc3s2)c1] | 2-(3-(3-methoxyphenyl)-1- methylureido)-5-oxo-5H- thieno[3,2-b]pyran-6- carboxylic acid | A |
| 4 | 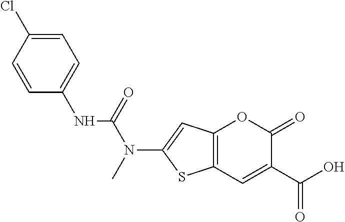 [SMILES: CN(C(=O)Nc1ccc(Cl)cc1)c1cc2oc(=O)c(C(=O)O)cc2s1] | 2-(3-(4-chlorophenyl)-1- methylureido)-5-oxo-5H- thieno[3,2-b]pyran-6- carboxylic acid | A |
| 5 | 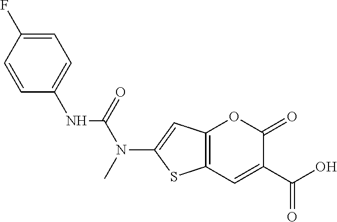 [SMILES: CN(C(=O)Nc1ccc(F)cc1)c1cc2oc(=O)c(C(=O)O)cc2s1] | 2-(3-(4-fluorophenyl)-1- methylureido)-5-oxo-5H- thieno[3,2-b]pyran-6- carboxylic acid | A |
| 6 | 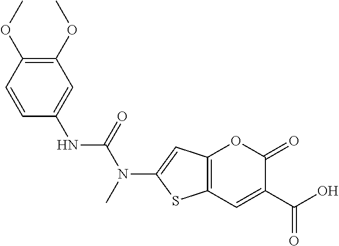 [SMILES: COc1ccc(NC(=O)N(C)c2cc3oc(=O)c(C(=O)O)cc3s2)cc1OC] | 2-(3-(3,4-dimethoxyphenyl)- 1-methylureido)-5-oxo-5H- thieno[3,2-b]pyran-6- carboxylic acid | A |
| 7 | 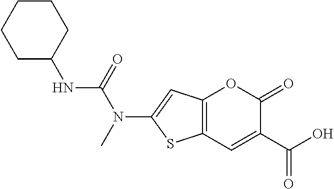 [SMILES: CN(C(=O)NC1CCCCC1)c1cc2oc(=O)c(C(=O)O)cc2s1] | 2-(3-cyclohexyl-1- methylureido)-5-oxo-5H- thieno[3,2-b]pyran-6- carboxylic acid | A |
| 8 | 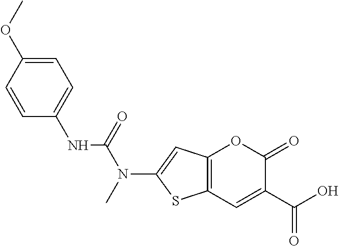 [SMILES: COc1ccc(NC(=O)N(C)c2cc3oc(=O)c(C(=O)O)cc3s2)cc1] | 2-(3-(4-methoxyphenyl)-1- methylureido)-5-oxo-5H- thieno[3,2-b]pyran-6- carboxylic acid | A |
| 9 | 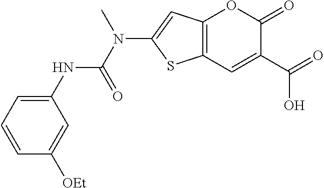 [SMILES: CCOc1cccc(NC(=O)N(C)c2cc3oc(=O)c(C(=O)O)cc3s2)c1] | 2-(3-(3-ethoxyphenyl)-1- methylureido)-5-oxo-5H- thieno[3,2-b]pyran-6- carboxylic acid | A |
| 10 | 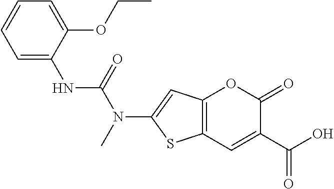 [SMILES: CCOc1ccccc1NC(=O)N(C)c1cc2oc(=O)c(C(=O)O)cc2s1] | 2-(3-(2-ethoxyphenyl)-1- methylureido)-5-oxo-5H- thieno[3,2-b]pyran-6- carboxylic acid | A |
| 11 | 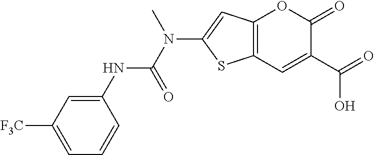 [SMILES: CN(C(=O)Nc1cccc(C(F)(F)F)c1)c1cc2oc(=O)c(C(=O)O)cc2s1] | 2-(1-methyl-3-(3- (trifluoromethyl)phenyl) ureido)- 5-oxo-5H-thieno[3,2- b]pyran-6-carboxylic acid | A |
| 12 | 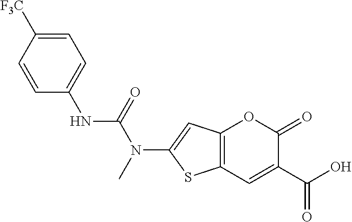 [SMILES: CN(C(=O)Nc1ccc(C(F)(F)F)cc1)c1cc2oc(=O)c(C(=O)O)cc2s1] | 2-(1-methyl-3-(4- (trifluoromethyl)phenyl) ureido)- 5-oxo-5H-thieno[3,2- b]pyran-6-carboxylic acid | A |
| 13 | 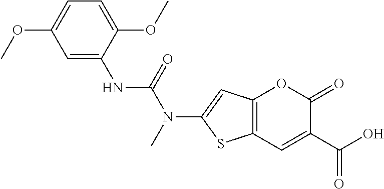 [SMILES: COc1ccc(OC)c(NC(=O)N(C)c2cc3oc(=O)c(C(=O)O)cc3s2)c1] | 2-(3-(2,5-dimethoxyphenyl)- 1-methylureido)-5-oxo-5H- thieno[3,2-b]pyran-6- carboxylic acid | A |
| 14 | 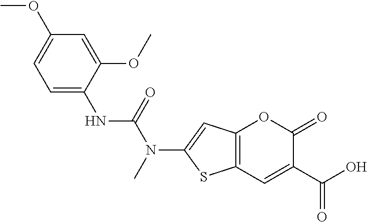 [SMILES: COc1ccc(NC(=O)N(C)c2cc3oc(=O)c(C(=O)O)cc3s2)c(OC)c1] | 2-(3-(2,4-dimethoxyphenyl)- 1-methylureido)-5-oxo-5H- thieno[3,2-b]pyran-6- carboxylic acid | A |
| 15 | 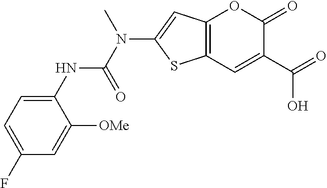 [SMILES: COc1cc(F)ccc1NC(=O)N(C)c1cc2oc(=O)c(C(=O)O)cc2s1] | 2-(3-(4-fluoro-2- methoxyphenyl)-1- methylureido)-5-oxo-5H- thieno[3,2-b]pyran-6- carboxylic acid | A |
| 16 | 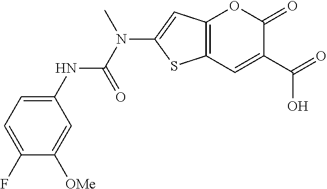 [SMILES: COc1cc(NC(=O)N(C)c2cc3oc(=O)c(C(=O)O)cc3s2)ccc1F] | 2-(3-(4-fluoro-3- methoxyphenyl)-1- methylureido)-5-oxo-5H- thieno[3,2-b]pyran-6- carboxylic acid | A |
| 17 | 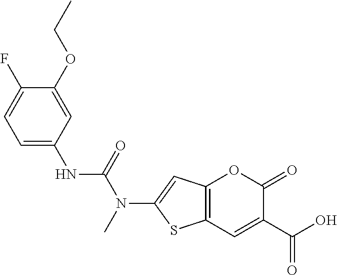 [SMILES: CCOc1cc(NC(=O)N(C)c2cc3oc(=O)c(C(=O)O)cc3s2)ccc1F] | 2-(3-(3-ethoxy-4- fluorophenyl)-1- methylureido)-5-oxo-5H- thieno[3,2-b]pyran-6- carboxylic acid | A |
| 18 | 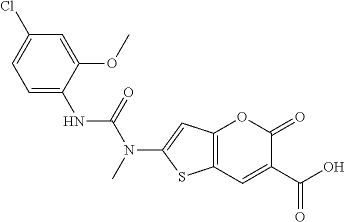 [SMILES: COc1cc(Cl)ccc1NC(=O)N(C)c1cc2oc(=O)c(C(=O)O)cc2s1] | 2-(3-(4-chloro-2- methoxyphenyl)-1- methylureido)-5-oxo-5H- thieno[3,2-b]pyran-6- carboxylic acid | A |
| 19 | 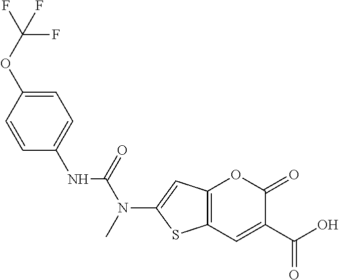 [SMILES: CN(C(=O)Nc1ccc(OC(F)(F)F)cc1)c1cc2oc(=O)c(C(=O)O)cc2s1] | 2-(1-methyl-3-(4- (trifluoromethoxy) phenyl)ureido)-5-oxo-5H- thieno[3,2-b]pyran-6- carboxylic acid | A |
| 20 | 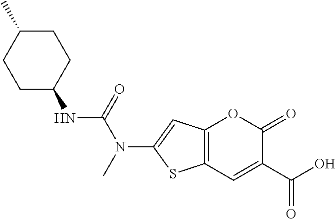 [SMILES: CN(C(=O)N[C@H]1CC[C@H](C)CC1)c1cc2oc(=O)c(C(=O)O)cc2s1] | 2-(1-methyl-3-((1r,4r)-4- methylcyclohexyl)ureido)-5- oxo-5H-thieno[3,2-b]pyran- 6-carboxylic acid | A |
| 21 | 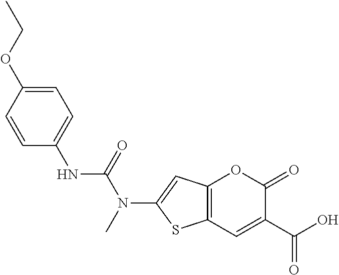 [SMILES: CCOc1ccc(NC(=O)N(C)c2cc3oc(=O)c(C(=O)O)cc3s2)cc1] | 2-(3-(4-ethoxyphenyl)-1- methylureido)-5-oxo-5H- thieno[3,2-b]pyran-6- carboxylic acid | A |
| 22 | 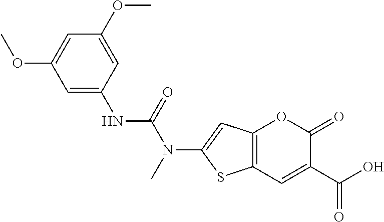 [SMILES: COc1cc(NC(=O)N(C)c2cc3oc(=O)c(C(=O)O)cc3s2)cc(OC)c1] | 2-(3-(3,5-dimethoxyphenyl)- 1-methylureido)-5-oxo-5H- thieno[3,2-b]pyran-6- carboxylic acid | A |
| 23 | 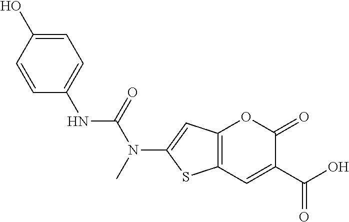 [SMILES: CN(C(=O)Nc1ccc(O)cc1)c1cc2oc(=O)c(C(=O)O)cc2s1] | 2-(3-(4-hydroxyphenyl)-1- methylureido)-5-oxo-5H- thieno[3,2-b]pyran-6- carboxylic acid | A |
| 24 | 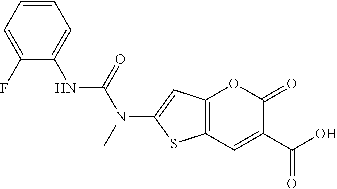 [SMILES: CN(C(=O)Nc1ccccc1F)c1cc2oc(=O)c(C(=O)O)cc2s1] | 2-(3-(2-fluorophenyl)-1- methylureido)-5-oxo-5H- thieno[3,2-b]pyran-6- carboxylic acid | A |
| 25 | 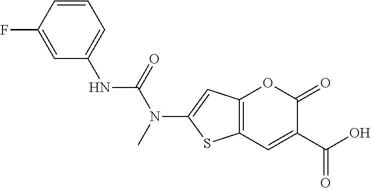 [SMILES: CN(C(=O)Nc1cccc(F)c1)c1cc2oc(=O)c(C(=O)O)cc2s1] | 2-(3-(3-fluorophenyl)-1- methylureido)-5-oxo-5H- thieno[3,2-b]pyran-6- carboxylic acid | A |
| 26 | 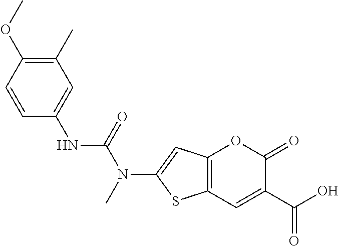 [SMILES: COc1ccc(NC(=O)N(C)c2cc3oc(=O)c(C(=O)O)cc3s2)cc1C] | 2-(3-(4-methoxy-3- methylphenyl)-1- methylureido)-5-oxo-5H- thieno[3,2-b]pyran-6- carboxylic acid | A |
| 27 | 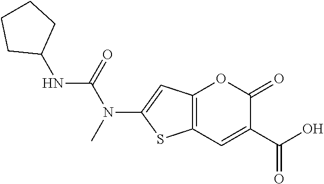 [SMILES: CN(C(=O)NC1CCCC1)c1cc2oc(=O)c(C(=O)O)cc2s1] | 2-(3-cyclopentyl-1- methylureido)-5-oxo-5H- thieno[3,2-b]pyran-6- carboxylic acid | A |
| 28 | 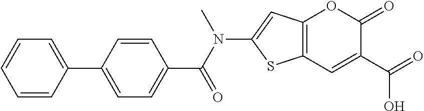 [SMILES: CN(C(=O)c1ccc(-c2ccccc2)cc1)c1cc2oc(=O)c(C(=O)O)cc2s1] | 2-(N-methyl-[1,1′-biphenyl]- 4-carboxamido)-5-oxo-5H- thieno[3,2-b]pyran-6- carboxylic acid | A |
| 29 | 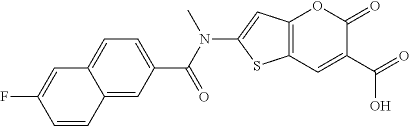 [SMILES: CN(C(=O)c1ccc2cc(F)ccc2c1)c1cc2oc(=O)c(C(=O)O)cc2s1] | 2-(6-fluoro-N-methyl-2- naphthamido)-5-oxo-5H- thieno[3,2-b]pyran-6- carboxylic acid | A |
| 30 | 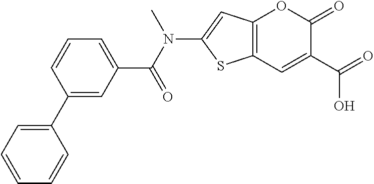 [SMILES: CN(C(=O)c1cccc(-c2ccccc2)c1)c1cc2oc(=O)c(C(=O)O)cc2s1] | 2-(N-methyl-[1,1′-biphenyl]- 3-carboxamido)-5-oxo-5H- thieno[3,2-b]pyran-6- carboxylic acid | A |
| 31 | 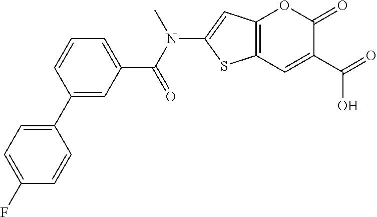 [SMILES: CN(C(=O)c1cccc(-c2ccc(F)cc2)c1)c1cc2oc(=O)c(C(=O)O)cc2s1] | 2-(4′-fluoro-N-methyl-[1,1′- biphenyl]-3-carboxamido)-5- oxo-5H-thieno[3,2-b]pyran- 6-carboxylic acid | A |
| 32 | 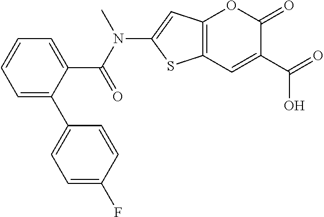 [SMILES: CN(C(=O)c1ccccc1-c1ccc(F)cc1)c1cc2oc(=O)c(C(=O)O)cc2s1] | 2-(4′-fluoro-N-methyl-[1,1′- biphenyl]-2-carboxamido)-5- oxo-5H-thieno[3,2-b]pyran- 6-carboxylic acid | A |
| 33 | 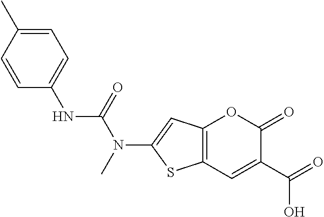 [SMILES: Cc1ccc(NC(=O)N(C)c2cc3oc(=O)c(C(=O)O)cc3s2)cc1] | 2-(1-methyl-3-(p- tolyl)ureido)-5-oxo-5H- thieno[3,2-b]pyran-6- carboxylic acid | A |
| 34 | 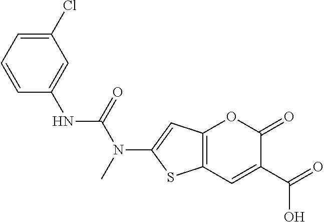 [SMILES: CN(C(=O)Nc1cccc(Cl)c1)c1cc2oc(=O)c(C(=O)O)cc2s1] | 2-(3-(3-chlorophenyl)-1- methylureido)-5-oxo-5H- thieno[3,2-b]pyran-6- carboxylic acid | A |
| 35 | 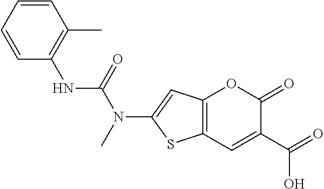 [SMILES: Cc1ccccc1NC(=O)N(C)c1cc2oc(=O)c(C(=O)O)cc2s1] | 2-(1-methyl-3-(o- tolyl)ureido)-5-oxo-5H- thieno[3,2-b]pyran-6- carboxylic acid | A |
| 36 | 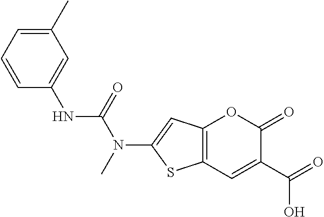 [SMILES: Cc1cccc(NC(=O)N(C)c2cc3oc(=O)c(C(=O)O)cc3s2)c1] | 2-(1-methyl-3-(m- tolyl)ureido)-5-oxo-5H- thieno[3,2-b]pyran-6- carboxylic acid | A |
| 37 | 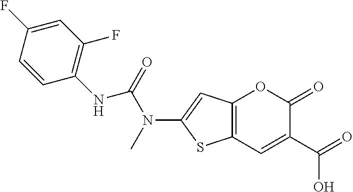 [SMILES: CN(C(=O)Nc1ccc(F)cc1F)c1cc2oc(=O)c(C(=O)O)cc2s1] | 2-(3-(2,4-difluorophenyl)-1- methylureido)-5-oxo-5H- thieno[3,2-b]pyran-6- carboxylic acid | A |
| 38 | 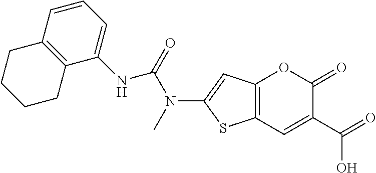 [SMILES: CN(C(=O)Nc1cccc2c1CCCC2)c1cc2oc(=O)c(C(=O)O)cc2s1] | 2-(1-methyl-3-(5,6,7,8- tetrahydronaphthalen-1- yl)ureido)-5-oxo-5H- thieno[3,2-b]pyran-6- carboxylic acid | A |
| 39 | 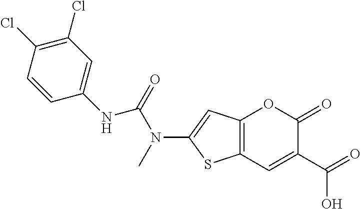 [SMILES: CN(C(=O)Nc1ccc(Cl)c(Cl)c1)c1cc2oc(=O)c(C(=O)O)cc2s1] | 2-(3-(3,4-difluorophenyl)-1- methylureido)-5-oxo-5H- thieno[3,2-b]pyran-6- carboxylic acid | A |
| 40 | 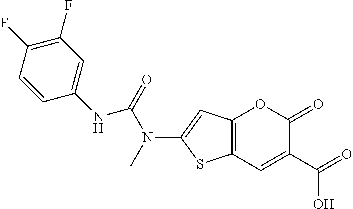 [SMILES: CN(C(=O)Nc1ccc(F)c(F)c1)c1cc2oc(=O)c(C(=O)O)cc2s1] | 2-(3-(3,4-difluorophenyl)-1- methylureido)-5-oxo-5H- thieno[3,2-b]pyran-6- carboxylic acid | A |
| 41 | 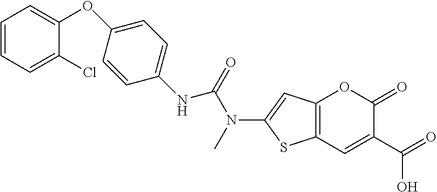 [SMILES: CN(C(=O)Nc1ccc(Oc2ccccc2Cl)cc1)c1cc2oc(=O)c(C(=O)O)cc2s1] | 2-(3-(4-(2- chlorophenoxy)phenyl)-1- methylureido)-5-oxo-5H- thieno[3,2-b]pyran-6- carboxylic acid | A |
| 42 | 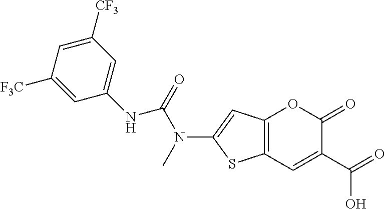 [SMILES: CN(C(=O)Nc1cc(C(F)(F)F)cc(C(F)(F)F)c1)c1cc2oc(=O)c(C(=O)O)cc2s1] | 2-(3-(3,5- bis(trifluoromethyl)phenyl)- 1-methylureido)-5-oxo-5H- thieno[3,2-b]pyran-6- carboxylic acid | A |
| 43 | 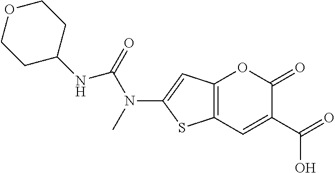 [SMILES: CN(C(=O)NC1CCOCC1)c1cc2oc(=O)c(C(=O)O)cc2s1] | 2-(1-methyl-3-(tetrahydro- 2H-pyran-4-yl)ureido)-5- oxo-5H-thieno[3,2-b]pyran- 6-carboxylic acid | A |
| 44 | 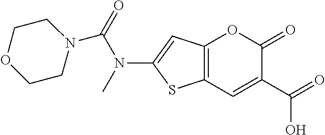 [SMILES: CN(C(=O)N1CCOCC1)c1cc2oc(=O)c(C(=O)O)cc2s1] | 2-(N-methylmorpholine-4- carboxamido)-5-oxo-5H- thieno[3,2-b]pyran-6- carboxylic acid | A |
| 45 | 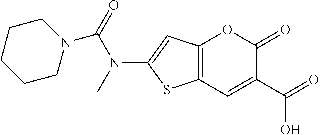 [SMILES: CN(C(=O)N1CCCCC1)c1cc2oc(=O)c(C(=O)O)cc2s1] | 2-(N-methylpiperidine-1- carboxamido)-5-oxo-5H- thieno[3,2-b]pyran-6- carboxylic acid | A |
| 46 | 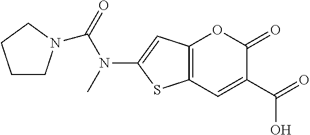 [SMILES: CN(C(=O)N1CCCC1)c1cc2oc(=O)c(C(=O)O)cc2s1] | 2-(N-methylpyrrolidine-1- carboxamido)-5-oxo-5H- thieno[3,2-b]pyran-6- carboxylic acid | A |
| — | 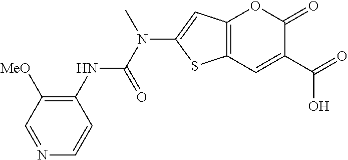 [SMILES: COc1cnccc1NC(=O)N(C)c1cc2oc(=O)c(C(=O)O)cc2s1] | 2-(3-(3-methoxypyridin-4- yl)-1-methylureido)-5-oxo- 5H-thieno[3,2-b]pyran-6- carboxylic acid | NT |
| — | 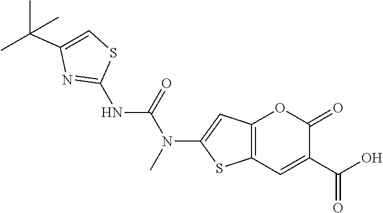 [SMILES: CN(C(=O)Nc1nc(C(C)(C)C)cs1)c1cc2oc(=O)c(C(=O)O)cc2s1] | 2-(3-(4-(tert-butyl)thiazol-2- yl)-1-methylureido)-5-oxo- 5H-thieno[3,2-b]pyran-6- carboxylic acid | NT |
| — | 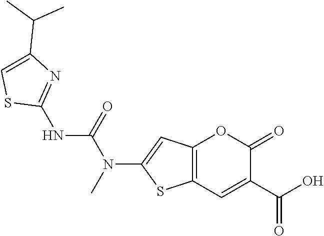 [SMILES: CC(C)c1csc(NC(=O)N(C)c2cc3oc(=O)c(C(=O)O)cc3s2)n1] | 2-(3-(4-isopropylthiazol-2- yl)-1-methylureido)-5-oxo- 5H-thieno[3,2-b]pyran-6- carboxylic acid | NT |
| — | 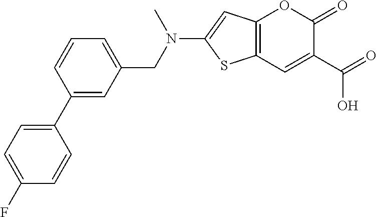 [SMILES: CN(Cc1cccc(-c2ccc(F)cc2)c1)c1cc2oc(=O)c(C(=O)O)cc2s1] | 2-(((4′-fluoro-[1,1′- biphenyl]-3- yl)methyl)(methyl)amino)-5- oxo-5H-thieno[3,2-b]pyran- 6-carboxylic acid | NT |
| — | 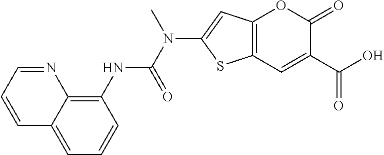 [SMILES: CN(C(=O)Nc1cccc2cccnc12)c1cc2oc(=O)c(C(=O)O)cc2s1] | 2-(1-methyl-3-(quinolin-8- yl)ureido)-5-oxo-5H- thieno[3,2-b]pyran-6- carboxylic acid | NT |
| — | 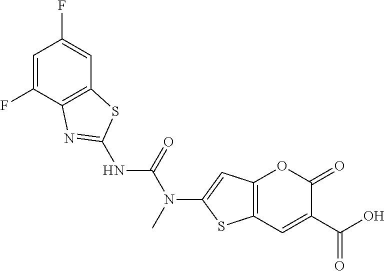 [SMILES: CN(C(=O)Nc1nc2c(F)cc(F)cc2s1)c1cc2oc(=O)c(C(=O)O)cc2s1] | 2-(3-(4,6- difluorobenzo[d]thiazol-2- yl)-1-methylureido)-5-oxo- 5H-thieno[3,2-b]pyran-6- carboxylic acid | NT |
| — | 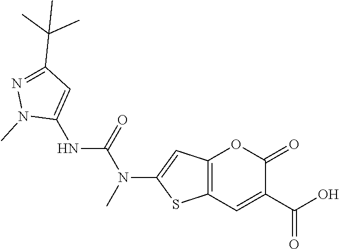 [SMILES: CN(C(=O)Nc1cc(C(C)(C)C)nn1C)c1cc2oc(=O)c(C(=O)O)cc2s1] | 2-(3-(3-(tert-butyl)-1-methyl- 1H-pyrazol-5-yl)-1- methylureido)-5-oxo-5H- thieno[3,2-b]pyran-6- carboxylic acid | NT |
| — | 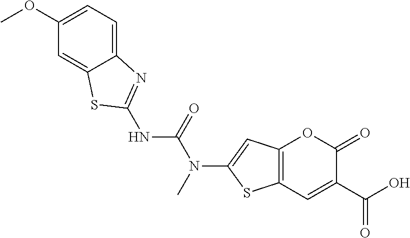 [SMILES: COc1ccc2nc(NC(=O)N(C)c3cc4oc(=O)c(C(=O)O)cc4s3)sc2c1] | 2-(3-(6- methoxybenzo[d]thiazol-2- yl)-1-methylureido)-5-oxo- 5H-thieno[3,2-b]pyran-6- carboxylic acid | NT |
| — | 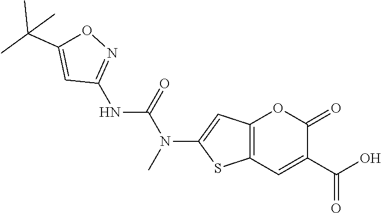 [SMILES: CN(C(=O)Nc1cc(C(C)(C)C)on1)c1cc2oc(=O)c(C(=O)O)cc2s1] | 2-(3-(5-(tert-butyl)isoxazol- 3-yl)-1-methylureido)-5-oxo- 5H-thieno[3,2-b]pyran-6- carboxylic acid | NT |
| — | 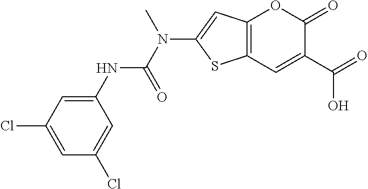 [SMILES: CN(C(=O)Nc1cc(Cl)cc(Cl)c1)c1cc2oc(=O)c(C(=O)O)cc2s1] | 2-(3-(3,5-dichlorophenyl)-1- methylureido)-5-oxo-5H- thieno[3,2-b]pyran-6- carboxylic acid | NT |
| — | 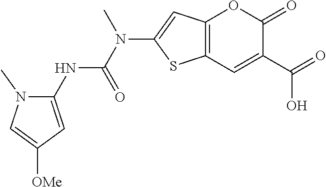 [SMILES: COc1cc(NC(=O)N(C)c2cc3oc(=O)c(C(=O)O)cc3s2)n(C)c1] | 2-(3-(4-methoxy-1-methyl- 1H-pyrrol-2-yl)-1- methylureido)-5-oxo-5H- thieno[3,2-b]pyran-6- carboxylic acid | NT |
| — | 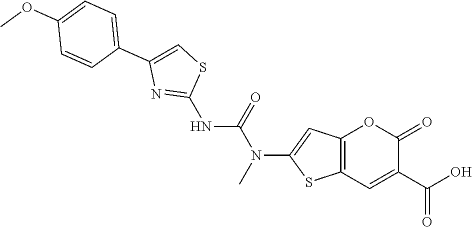 [SMILES: COc1ccc(-c2csc(NC(=O)N(C)c3cc4oc(=O)c(C(=O)O)cc4s3)n2)cc1] | 2-(3-(4-(4- methoxyphenyl)thiazol-2- yl)-1-methylureido)-5-oxo- 5H-thieno[3,2-b]pyran-6- carboxylic acid | NT |
| — | 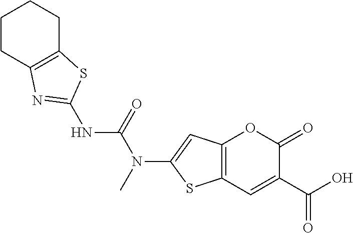 [SMILES: CN(C(=O)Nc1nc2c(s1)CCCC2)c1cc2oc(=O)c(C(=O)O)cc2s1] | 2-(1-methyl-3-(4,5,6,7- tetrahydrobenzo[d]thiazol-2- yl)ureido)-5-oxo-5H- thieno[3,2-b]pyran-6- carboxylic acid | NT |
| — | 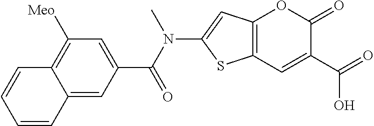 | 2-(4-methoxy-N-methyl-2- naphthamido)-5-oxo-5H- thieno[3,2-b]pyran-6- carboxylic acid | NT |
| — | 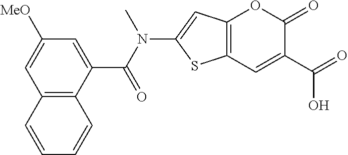 [SMILES: COc1cc(C(=O)N(C)c2cc3oc(=O)c(C(=O)O)cc3s2)c2ccccc2c1] | 2-(3-methoxy-N-methyl-1- naphthamido)-5-oxo-5H- thieno[3,2-b]pyran-6- carboxylic acid | NT |
| — | 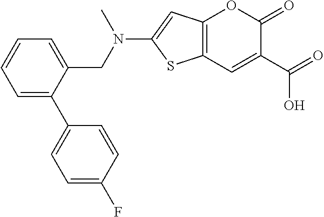 [SMILES: CN(Cc1ccccc1-c1ccc(F)cc1)c1cc2oc(=O)c(C(=O)O)cc2s1] | 2-(((4′-fluoro-[1,1′- biphenyl]-2- yl)methyl)(methyl)amino)-5- oxo-5H-thieno[3,2-b]pyran- 6-carboxylic acid | NT |
| — | 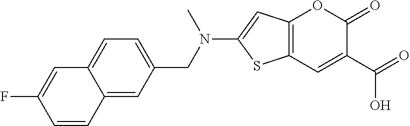 [SMILES: CN(Cc1ccc2cc(F)ccc2c1)c1cc2oc(=O)c(C(=O)O)cc2s1] | 2-(((6-fluoronaphthalen-2- yl)methyl)(methyl)amino)-5- oxo-5H-thieno[3,2-b]pyran- 6-carboxylic acid | NT |
| — | 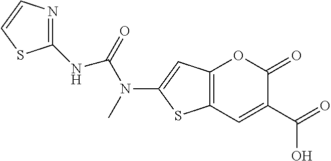 [SMILES: CN(C(=O)Nc1nccs1)c1cc2oc(=O)c(C(=O)O)cc2s1] | 2-(1-methyl-3-(thiazol-2- yl)ureido)-5-oxo-5H- thieno[3,2-b]pyran-6- carboxylic acid | NT |
| — | 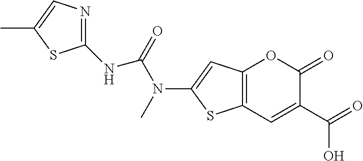 [SMILES: Cc1cnc(NC(=O)N(C)c2cc3oc(=O)c(C(=O)O)cc3s2)s1] | 2-(1-methyl-3-(5- methylthiazol-2-yl)ureido)-5- oxo-5H-thieno[3,2-b]pyran- 6-carboxylic acid | NT |
| — | 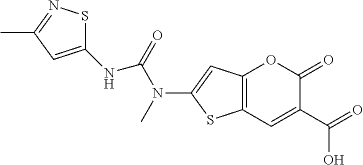 [SMILES: Cc1cc(NC(=O)N(C)c2cc3oc(=O)c(C(=O)O)cc3s2)sn1] | 2-(1-methyl-3-(3- methylisothiazol-5- yl)ureido)-5-oxo-5H- thieno[3,2-b]pyran-6- carboxylic acid | NT |
| — | 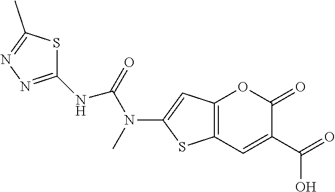 [SMILES: Cc1nnc(NC(=O)N(C)c2cc3oc(=O)c(C(=O)O)cc3s2)s1] | 2-(1-methyl-3-(5-methyl- 1,3,4-thiadiazol-2-yl)ureido)- 5-oxo-5H-thieno[3,2- b]pyran-6-carboxylic acid | NT |
| — | 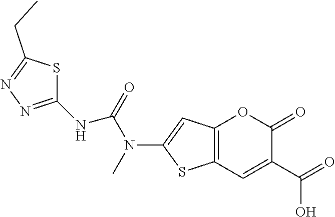 [SMILES: CCc1nnc(NC(=O)N(C)c2cc3oc(=O)c(C(=O)O)cc3s2)s1] | 2-(3-(5-ethyl-1,3,4- thiadiazol-2-yl)-1- methylureido)-5-oxo-5H- thieno[3,2-b]pyran-6- carboxylic acid | NT |
| — | 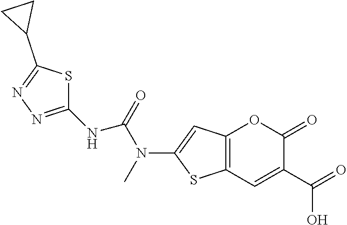 [SMILES: CN(C(=O)Nc1nnc(C2CC2)s1)c1cc2oc(=O)c(C(=O)O)cc2s1] | 2-(3-(5-cyclopropyl-1,3,4- thiadiazol-2-yl)-1- methylureido)-5-oxo-5H- thieno[3,2-b]pyran-6- carboxylic acid | NT |
| — | 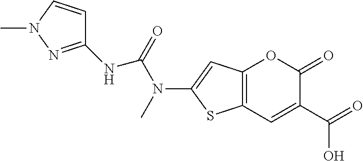 [SMILES: CN(C(=O)Nc1ccn(C)n1)c1cc2oc(=O)c(C(=O)O)cc2s1] | 2-(1-methyl-3-(1-methyl-1H- pyrazol-3-yl)ureido)-5-oxo- 5H-thieno[3,2-b]pyran-6- carboxylic acid | NT |
| — | 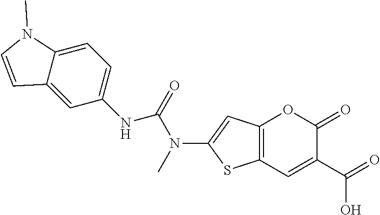 [SMILES: CN(C(=O)Nc1ccc2c(ccn2C)c1)c1cc2oc(=O)c(C(=O)O)cc2s1] | 2-(1-methyl-3-(1-methyl-1H- indol-5-yl)ureido)-5-oxo-5H- thieno[3,2-b]pyran-6- carboxylic acid | NT |
| — | 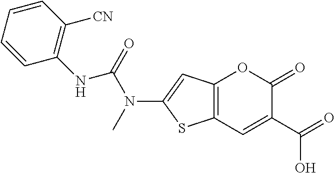 [SMILES: CN(C(=O)Nc1ccccc1C#N)c1cc2oc(=O)c(C(=O)O)cc2s1] | 2-(3-(2-cyanophenyl)-1- methylureido)-5-oxo-5H- thieno[3,2-b]pyran-6- carboxylic acid | NT |
| — | 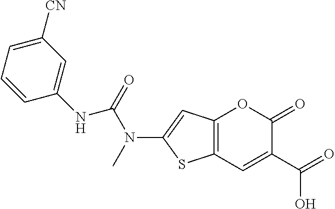 [SMILES: CN(C(=O)Nc1cccc(C#N)c1)c1cc2oc(=O)c(C(=O)O)cc2s1] | 2-(3-(3-cyanophenyl)-1- methylureido)-5-oxo-5H- thieno[3,2-b]pyran-6- carboxylic acid | NT |
| — | 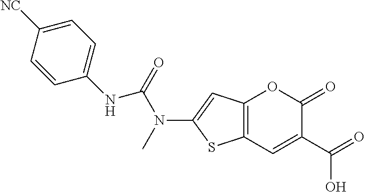 [SMILES: CN(C(=O)Nc1ccc(C#N)cc1)c1cc2oc(=O)c(C(=O)O)cc2s1] | 2-(3-(4-cyanophenyl)-1- methylureido)-5-oxo-5H- thieno[3,2-b]pyran-6- carboxylic acid | NT |
| — | 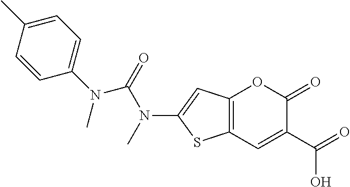 [SMILES: Cc1ccc(N(C)C(=O)N(C)c2cc3oc(=O)c(C(=O)O)cc3s2)cc1] | 2-(1,3-dimethyl-3-(p- tolyl)ureido)-5-oxo-5H- thieno[3,2-b]pyran-6- carboxylic acid | NT |
| — | 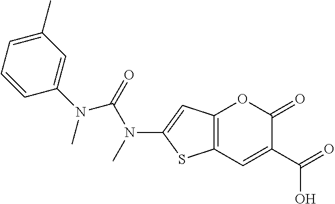 [SMILES: Cc1cccc(N(C)C(=O)N(C)c2cc3oc(=O)c(C(=O)O)cc3s2)c1] | 2-(1,3-dimethyl-3-(m- tolyl)ureido)-5-oxo-5H- thieno[3,2-b]pyran-6- carboxylic acid | NT |
| — | 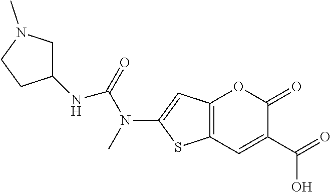 [SMILES: CN1CCC(NC(=O)N(C)c2cc3oc(=O)c(C(=O)O)cc3s2)C1] | 2-(1-methyl-3-(1- methylpyrrolidin-3- yl)ureido)-5-oxo-5H- thieno[3,2-b]pyran-6- carboxylic acid | NT |
| — | 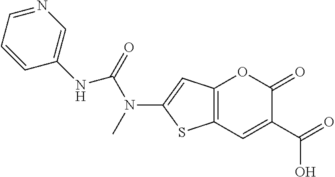 [SMILES: CN(C(=O)Nc1cccnc1)c1cc2oc(=O)c(C(=O)O)cc2s1] | 2-(1-methyl-3-(pyridin-3- yl)ureido)-5-oxo-5H- thieno[3,2-b]pyran-6- carboxylic acid | NT |
| — | 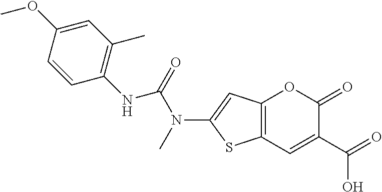 [SMILES: COc1ccc(NC(=O)N(C)c2cc3oc(=O)c(C(=O)O)cc3s2)c(C)c1] | 2-(3-(4-methoxy-2- methylphenyl)-1- methylureido)-5-oxo-5H- thieno[3,2-b]pyran-6- carboxylic acid | NT |
| — | 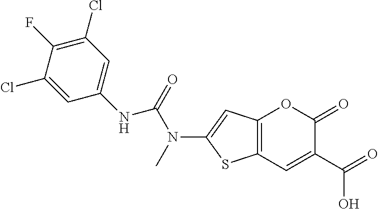 [SMILES: CN(C(=O)Nc1cc(Cl)c(F)c(Cl)c1)c1cc2oc(=O)c(C(=O)O)cc2s1] | 2-(3-(3,5-dichloro-4- fluorophenyl)-1- methylureido)-5-oxo-5H- thieno[3,2-b]pyran-6- carboxylic acid | NT |
| — | 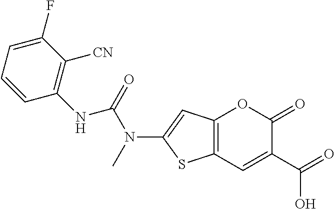 [SMILES: CN(C(=O)Nc1cccc(F)c1C#N)c1cc2oc(=O)c(C(=O)O)cc2s1] | 2-(3-(2-cyano-3- fluorophenyl)-1- methylureido)-5-oxo-5H- thieno[3,2-b]pyran-6- carboxylic acid | NT |
| — | 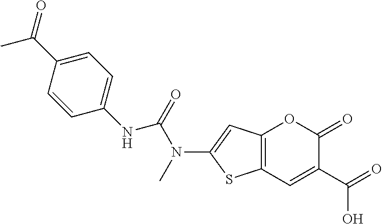 [SMILES: CC(=O)c1ccc(NC(=O)N(C)c2cc3oc(=O)c(C(=O)O)cc3s2)cc1] | 2-(3-(4-acetylphenyl)-1- methylureido)-5-oxo-5H- thieno[3,2-b]pyran-6- carboxylic acid | NT |
| — | 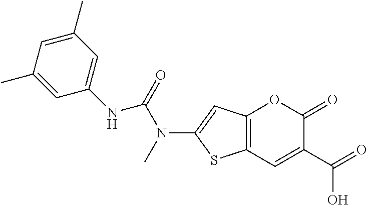 [SMILES: Cc1cc(C)cc(NC(=O)N(C)c2cc3oc(=O)c(C(=O)O)cc3s2)c1] | 2-(3-(3,5-dimethylphenyl)-1- methylureido)-5-oxo-5H- thieno[3,2-b]pyran-6- carboxylic acid | NT |
| — | 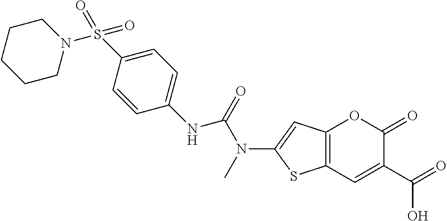 [SMILES: CN(C(=O)Nc1ccc(S(=O)(=O)N2CCCCC2)cc1)c1cc2oc(=O)c(C(=O)O)cc2s1] | 2-(1-methyl-3-(4-(piperidin- 1-ylsulfonyl)phenyl)ureido)- 5-oxo-5H-thieno[3,2- b]pyran-6-carboxylic acid | NT |
| — | 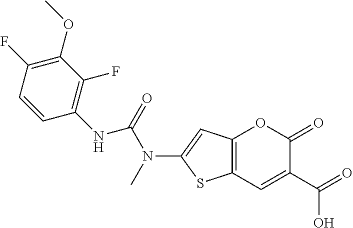 [SMILES: COc1c(F)ccc(NC(=O)N(C)c2cc3oc(=O)c(C(=O)O)cc3s2)c1F] | 2-(3-(2,4-difluoro-3- methoxyphenyl)-1- methylureido)-5-oxo-5H- thieno[3,2-b]pyran-6- carboxylic acid | NT |
| — | 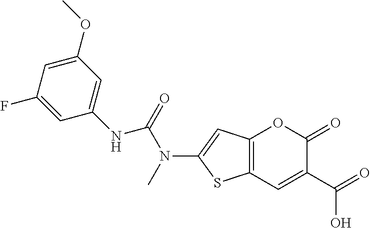 [SMILES: COc1cc(F)cc(NC(=O)N(C)c2cc3oc(=O)c(C(=O)O)cc3s2)c1] | 2-(3-(3-fluoro-5- methoxyphenyl)-1- methylureido)-5-oxo-5H- thieno[3,2-b]pyran-6- carboxylic acid | NT |
| — | 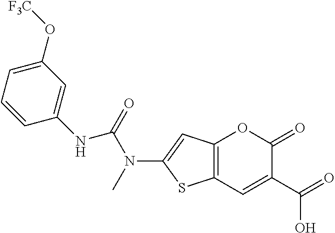 [SMILES: CN(C(=O)Nc1cccc(OC(F)(F)F)c1)c1cc2oc(=O)c(C(=O)O)cc2s1] | 2-(1-methyl-3-(3- (trifluoromethoxy)phenyl) ureido)-5-oxo-5H-thieno[3,2- b]pyran-6-carboxylic acid | NT |
| — | 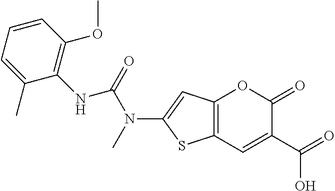 [SMILES: COc1cccc(C)c1NC(=O)N(C)c1cc2oc(=O)c(C(=O)O)cc2s1] | 2-(3-(2-methoxy-6- methylphenyl)-1- methylureido)-5-oxo-5H- thieno[3,2-b]pyran-6- carboxylic acid | NT |
| — | 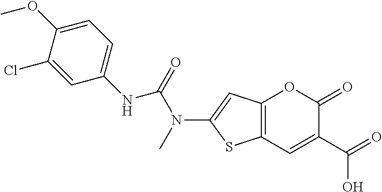 [SMILES: COc1ccc(NC(=O)N(C)c2cc3oc(=O)c(C(=O)O)cc3s2)cc1Cl] | 2-(3-(3-chloro-4- methoxyphenyl)-1- methylureido)-5-oxo-5H- thieno[3,2-b]pyran-6- carboxylic acid | NT |
| — | 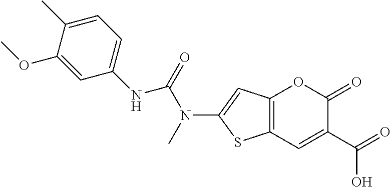 [SMILES: COc1cc(NC(=O)N(C)c2cc3oc(=O)c(C(=O)O)cc3s2)ccc1C] | 2-(3-(3-methoxy-4- methylphenyl)-1- methylureido)-5-oxo-5H- thieno[3,2-b]pyran-6- carboxylic acid | NT |
| — | 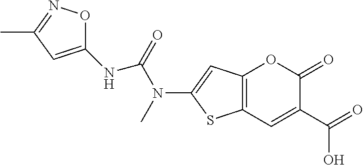 [SMILES: Cc1cc(NC(=O)N(C)c2cc3oc(=O)c(C(=O)O)cc3s2)on1] | 2-(1-methyl-3-(3- methylisoxazol-5-yl)ureido)- 5-oxo-5H-thieno[3,2- b]pyran-6-carboxylic acid | NT |
| — | 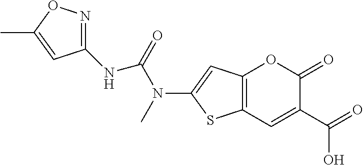 [SMILES: Cc1cc(NC(=O)N(C)c2cc3oc(=O)c(C(=O)O)cc3s2)no1] | 2-(1-methyl-3-(5- methylisoxazol-3-yl)ureido)- 5-oxo-5H-thieno[3,2- b]pyran-6-carboxylic acid | NT |
| — | 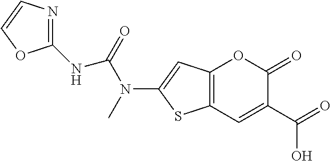 [SMILES: CN(C(=O)Nc1ncco1)c1cc2oc(=O)c(C(=O)O)cc2s1] | 2-(1-methyl-3-(oxazol-2- yl)ureido)-5-oxo-5H- thieno[3,2-b]pyran-6- carboxylic acid | NT |
| — | 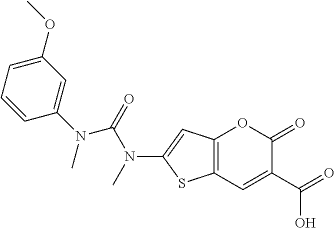 [SMILES: COc1cccc(N(C)C(=O)N(C)c2cc3oc(=O)c(C(=O)O)cc3s2)c1] | 2-(3-(3-methoxyphenyl)-1,3- dimethylureido)-5-oxo-5H- thieno[3,2-b]pyran-6- carboxylic acid | NT |
| — | 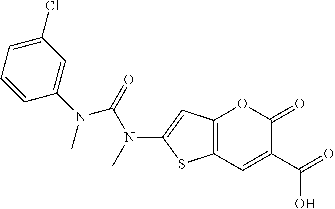 [SMILES: CN(C(=O)N(C)c1cc2oc(=O)c(C(=O)O)cc2s1)c1cccc(Cl)c1] | 2-(3-(3-chlorophenyl)-1,3- dimethylureido)-5-oxo-5H- thieno[3,2-b]pyran-6- carboxylic acid | NT |
| — | 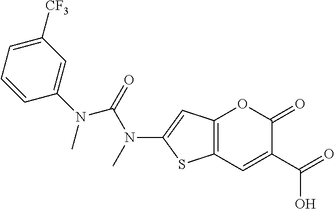 [SMILES: CN(C(=O)N(C)c1cc2oc(=O)c(C(=O)O)cc2s1)c1cccc(C(F)(F)F)c1] | 2-(1,3-dimethyl-3-(3- (trifluoromethyl)phenyl) ureido)- 5-oxo-5H-thieno[3,2- b]pyran-6-carboxylic acid | NT |
| — | 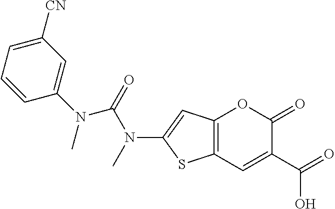 [SMILES: CN(C(=O)N(C)c1cc2oc(=O)c(C(=O)O)cc2s1)c1cccc(C#N)c1] | 2-(3-(3-cyanophenyl)-1,3- dimethylureido)-5-oxo-5H- thieno[3,2-b]pyran-6- carboxylic acid | NT |
| — | 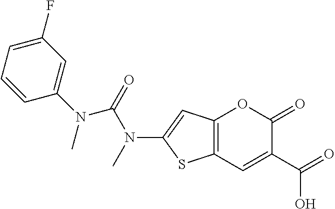 [SMILES: CN(C(=O)N(C)c1cc2oc(=O)c(C(=O)O)cc2s1)c1cccc(F)c1] | 2-(3-(3-fluorophenyl)-1,3- dimethylureido)-5-oxo-5H- thieno[3,2-b]pyran-6- carboxylic acid | NT |
| — | 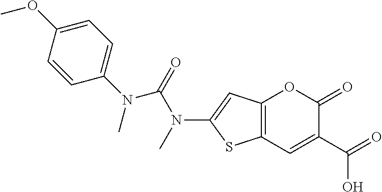 [SMILES: COc1ccc(N(C)C(=O)N(C)c2cc3oc(=O)c(C(=O)O)cc3s2)cc1] | 2-(3-(4-methoxyphenyl)-1,3- dimethylureido)-5-oxo-5H- thieno[3,2-b]pyran-6- carboxylic acid | NT |
| — | 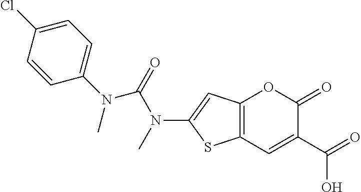 [SMILES: CN(C(=O)N(C)c1cc2oc(=O)c(C(=O)O)cc2s1)c1ccc(Cl)cc1] | 2-(3-(4-chlorophenyl)-1,3- dimethylureido)-5-oxo-5H- thieno[3,2-b]pyran-6- carboxylic acid | NT |
| — | 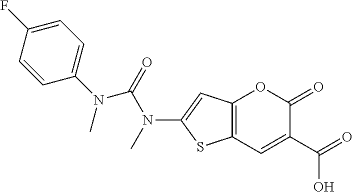 [SMILES: CN(C(=O)N(C)c1cc2oc(=O)c(C(=O)O)cc2s1)c1ccc(F)cc1] | 2-(3-(4-fluorophenyl)-1,3- dimethylureido)-5-oxo-5H- thieno[3,2-b]pyran-6- carboxylic acid | NT |
| — | 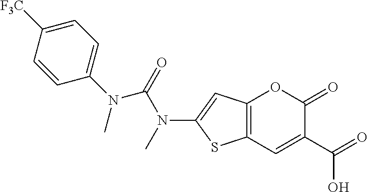 [SMILES: CN(C(=O)N(C)c1cc2oc(=O)c(C(=O)O)cc2s1)c1ccc(C(F)(F)F)cc1] | 2-(1,3-dimethyl-3-(4- (trifluoromethyl)phenyl) ureido)- 5-oxo-5H-thieno[3,2- b]pyran-6-carboxylic acid | NT |
| — | 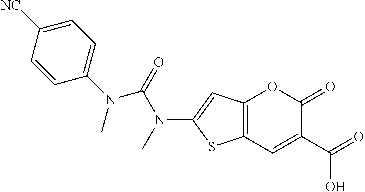 [SMILES: CN(C(=O)N(C)c1cc2oc(=O)c(C(=O)O)cc2s1)c1ccc(C#N)cc1] | 2-(3-(4-cyanophenyl)-1,3- dimethylureido)-5-oxo-5H- thieno[3,2-b]pyran-6- carboxylic acid | NT |
| — | 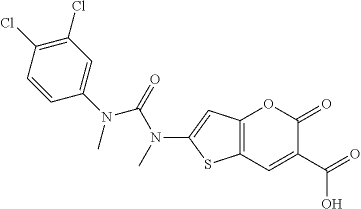 [SMILES: CN(C(=O)N(C)c1cc2oc(=O)c(C(=O)O)cc2s1)c1ccc(Cl)c(Cl)c1] | 2-(3-(3,4-dichlorophenyl)- 1,3-dimethylureido)-5-oxo- 5H-thieno[3,2-b]pyran-6- carboxylic acid | NT |
| — | 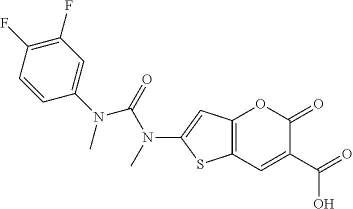 [SMILES: CN(C(=O)N(C)c1cc2oc(=O)c(C(=O)O)cc2s1)c1ccc(F)c(F)c1] | 2-(3-(3,4-difluorophenyl)- 1,3-dimethylureido)-5-oxo- 5H-thieno[3,2-b]pyran-6- carboxylic acid | NT |
| — | 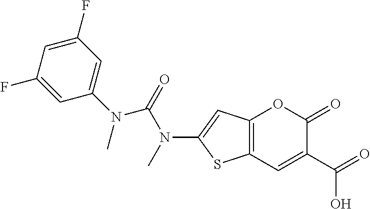 [SMILES: CN(C(=O)N(C)c1cc2oc(=O)c(C(=O)O)cc2s1)c1cc(F)cc(F)c1] | 2-(3-(3,5-difluorophenyl)- 1,3-dimethylureido)-5-oxo- 5H-thieno[3,2-b]pyran-6- carboxylic acid | NT |
| — | 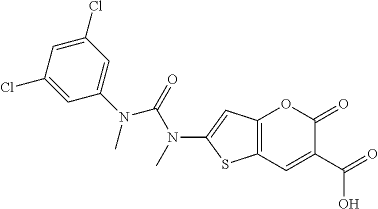 [SMILES: CN(C(=O)N(C)c1cc2oc(=O)c(C(=O)O)cc2s1)c1cc(Cl)cc(Cl)c1] | 2-(3-(3,5-dichlorophenyl)- 1,3-dimethylureido)-5-oxo- 5H-thieno[3,2-b]pyran-6- carboxylic acid | NT |
| — | 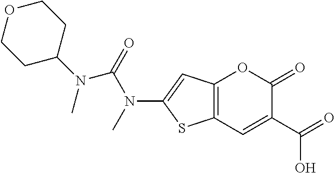 [SMILES: CN(C(=O)N(C)C1CCOCC1)c1cc2oc(=O)c(C(=O)O)cc2s1] | 2-(1,3-dimethyl-3- (tetrahydro-2H-pyran-4- yl)ureido)-5-oxo-5H- thieno[3,2-b]pyran-6- carboxylic acid | NT |
| — | 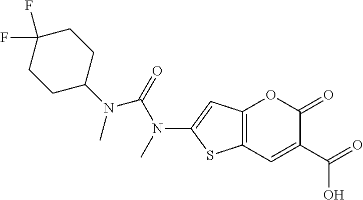 [SMILES: CN(C(=O)N(C)C1CCC(F)(F)CC1)c1cc2oc(=O)c(C(=O)O)cc2s1] | 2-(3-(4,4- difluorocyclohexyl)-1,3- dimethylureido)-5-oxo-5H- thieno[3,2-b]pyran-6- carboxylic acid | NT |
| — | 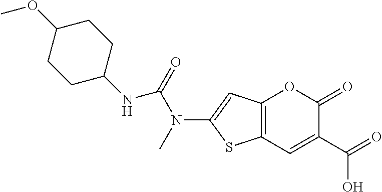 [SMILES: COC1CCC(NC(=O)N(C)c2cc3oc(=O)c(C(=O)O)cc3s2)CC1] | 2-(3-(4-methoxycyclohexyl)- 1-methylureido)-5-oxo-5H- thieno[3,2-b]pyran-6- carboxylic acid | NT |
| — | 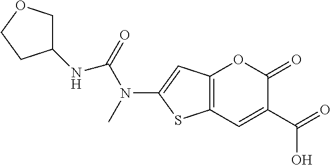 [SMILES: CN(C(=O)NC1CCOC1)c1cc2oc(=O)c(C(=O)O)cc2s1] | 2-(1-methyl-3- (tetrahydrofuran-3- yl)ureido)-5-oxo-5H- thieno[3,2-b]pyran-6- carboxylic acid | NT |

IC₅₀; A = <1 uM; B = 1-10 uM; C = >10 uM; NT = Not Tested

### Example 48: Lactate Detection Assay in Tumor Cell Lines

The inhibition of monocarboxylate transporters of the invention was measured and data are shown in Table II. Cells are maintained in their appropriate growth medium (RPMI medium with 2 g/L glucose, 2 mM L-glutamine supplemented with 10% FBS and P/S (growth medium). 15,000-25,000 cells were seeded into white 96-well plates in growth medium and incubated for 24 hours at 37° C. and 5% CO₂. A duplicate plate was also seeded for normalization by an MTS assay. Dry weight compound stocks were dissolved to a concentration of 10 mM in 100% DMSO. Compounds were further diluted in the assay medium (Lactate media: 10 mM lactate, 5% FBS, and 1×P/S; Glucose media: RPMI, 5% FBS, and 1×P/S). Growth media was changed 24 hours later to assay medium containing 10 μM compound or vehicle (DMSO) control and incubated for 24 hours. Conditioned media was collected and the cells were washed in 200 μL ice-cold PBS. Cells were lysed in 37.5 μL Inactivation solution (25 μL PBS+12.5 μL 0.6N HCl; 0.25% DTAB) which rapidly inhibits cell metabolism, destroys reduced NAD(P)H dinucleotides and inhibits activity of endogenous proteins. After a 5 minute incubation, 12.5 μL Neutralization solution (1 M Trizma) is incubated for 1 minute. Intracellular lactate measurements were performed using a Lactate-glo kit (Promega). Briefly, the lactate detection reagent is mixed immediately before use and 50 μL is pipetted into each well and incubated at room temperature for 1 hour. Lactate is oxidized by enzymatic reactions to generate light. The luminescence is recorded using a Spectramax M5e plate reader and the concentration of lactate is determined using a known concentration of spiked lactate in PBS using GraphPad Prism. Assay data of selected compounds are listed in Table 2, where IC₅₀: A=<1 uM.

TABLE 2
Lactate Detection Assay Results

| | | Lactate Detection Assay |
|---|---|---|
| Example | Structure | (IC₅₀: μM) |
| 7 | 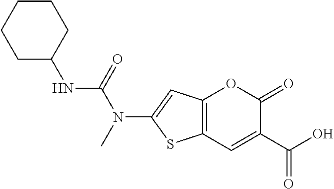 [SMILES: CN(C(=O)NC1CCCCC1)c1cc2oc(=O)c(C(=O)O)cc2s1] | A |
| 10 | 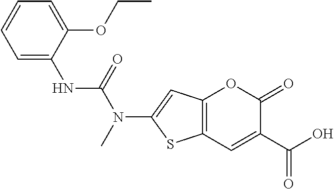 [SMILES: CCOc1ccccc1NC(=O)N(C)c1cc2oc(=O)c(C(=O)O)cc2s1] | A |

IC₅₀: A = <1 um

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound represented by formula (I): 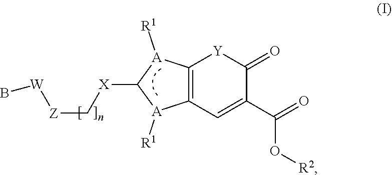
or a pharmaceutically acceptable salt thereof, wherein:
subscript n is 0, 1, or 2;
W is O, NH, or NR″;
X is O or NR″;
Y is O or NR″;
Z is a bond, CH₂, C═O, SO₂; 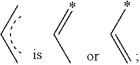
each A is independently selected from the group consisting of N, NR″, S, O, CR″ and CHR″;
each R¹ is independently absent or selected from the group consisting of hydrogen, halogen, C₁₋₆ alkyl, CHF₂, CF₃, CN, —C(O)R″, —C(O)OR″, —SO₂R″, —C(O)NR″₂, —C(O)N(OR″)R″ and —C≡CH;
R² is selected from the group consisting of:
hydrogen;
—C(O)R″;
—(CH₂)₀₋₄C(O)R″;
—(CH₂)₀₋₄C(O)OR″;
optionally substituted C₁₋₆ alkyl;
an optionally substituted 3-8 membered saturated or partially unsaturated cycloalkyl ring;
an optionally substituted 3-8 membered saturated or partially unsaturated heterocycloalkyl ring having 1-2 heteroatoms independently selected from nitrogen, oxygen, and sulfur;
optionally substituted phenyl; and
an optionally substituted 5-6 membered heteroaryl ring having 1-4 heteroatoms independently selected from nitrogen, oxygen, and sulfur;
B is a ring selected from the group consisting of:
a 3-8 membered saturated or partially unsaturated monocyclic carbocyclic ring, phenyl,
a 8-10 membered bicyclic aryl ring,
a 3-8 membered saturated or partially unsaturated monocyclic or bicyclic heterocyclic ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
a 8-10 membered bicyclic heteroaryl ring having 1-4 heteroatoms independently selected from nitrogen, oxygen, and sulfur,
wherein B is optionally substituted with one or more substituents selected from R′ and R″;
R′ is selected from the group consisting of OH, C₁₋₆ haloalkyl, C₁₋₆ alkoxy, C₁₋₆ haloalkoxy, and O-phenyl optionally substituted with halogen, C₁₋₆ alkyl, or C₁₋₆ alkoxy;
R″ is selected from the group consisting of:
R¹;
a 3-8 membered saturated or partially unsaturated cycloalkyl ring, optionally substituted with halogen or C₁₋₆ alkyl;
a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
phenyl optionally substituted with halogen, C₁₋₆ alkyl, or C₁₋₆ alkoxy; and
a 5-6 membered heteroaryl ring having 1-4 heteroatoms independently selected from nitrogen, oxygen, and sulfur, said ring optionally substituted with halogen or C₁₋₆ alkyl.

2. The compound of claim 1, wherein:
subscript n is 0, 1, or 2;
W is O, NH, or NR″;
X is O or NR″;
Y is O or NR″;
Z is a bond, CH₂, C═O, SO₂; 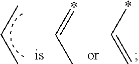
each A is independently selected from the group consisting of N, NR″, S, O, CR″ and CHR″;
R¹, when present, is selected from the group consisting of hydrogen, halogen, C₁₋₆ alkyl, CHF₂, CF₃, CN, —C(O)R″, —C(O)OR″, —SO₂R″, —C(O)NR″₂, —C(O)N(OR″)R″ and —C≡CH;
R² is selected from the group consisting of:
hydrogen;
—C(O)R″;
—(CH₂)₀₋₄C(O)R″;
—(CH₂)₀₋₄C(O)OR″;
optionally substituted C₁₋₆ alkyl;
an optionally substituted 3-8 membered saturated or partially unsaturated cycloalkyl ring;
an optionally substituted 3-8 membered saturated or partially unsaturated heterocycloalkyl ring having 1-2 heteroatoms independently selected from nitrogen, oxygen, and sulfur;
optionally substituted phenyl; and
an optionally substituted 5-6 membered heteroaryl ring having 1-4 heteroatoms independently selected from nitrogen, oxygen, and sulfur;
B is a ring selected from the group consisting of:
a 3-8 membered saturated or partially unsaturated monocyclic carbocyclic ring;
phenyl;
a 8-10 membered bicyclic aryl ring;
a 3-8 membered saturated or partially unsaturated monocyclic or bicyclic heterocyclic ring having 1-2 heteroatoms independently selected from nitrogen, oxygen, and sulfur;
a 5-6 membered monocyclic heteroaryl ring having 1-4 heteroatoms independently selected from nitrogen, oxygen, and sulfur; and
a 8-10 membered bicyclic heteroaryl ring having 1-4 heteroatoms independently selected from nitrogen, oxygen, and sulfur,
wherein B is optionally substituted with one or more R″ substituents;
R″ is selected from the group consisting of:
R¹;
a 3-8 membered saturated or partially unsaturated cycloalkyl ring, optionally substituted with halogen or C₁₋₆ alkyl;
a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
phenyl optionally substituted with halogen or C₁₋₆ alkyl; and
a 5-6 membered heteroaryl ring having 1-4 heteroatoms independently selected from nitrogen, oxygen, and sulfur, said ring optionally substituted with halogen or C₁₋₆ alkyl.

3. The compound of claims 1 or 2, wherein the compound of formula (I) is represented by formula (Ia):

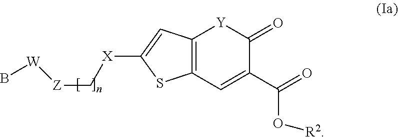

4. The compound of any one of claims 1 to 3, wherein subscript n is 0; Y is O; and R² is hydrogen.

5. The compound of any one of claims 1 to 4, wherein the compound of formula (I) is represented by formula (II):

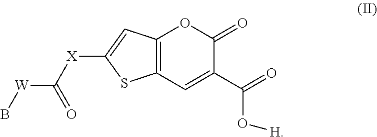

6. The compound of any one of claims 1 to 4, wherein the compound of formula (I) is represented by formula (III):

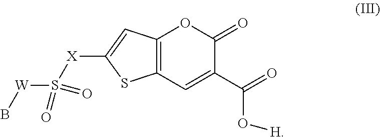

7. The compound of any one of claims 1 to 6, wherein X is O, NH or NMe.

8. The compound of any one of claims 1 to 7, wherein W is NH or NMe.

9. The compound of any one of claims 1, and 3 to 8, wherein B is a ring selected from the group consisting of:
a 5-6 membered saturated or partially unsaturated monocyclic carbocyclic ring, phenyl,
a 8-10 membered bicyclic aryl ring,
a 5-8 membered saturated or partially unsaturated monocyclic or bicyclic heterocyclic ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
a 8-10 membered bicyclic heteroaryl ring having 1-4 heteroatoms independently selected from nitrogen, oxygen, and sulfur,
wherein B is optionally substituted with one or more substituents selected from R′ and R″.

10. The compound of claim 9, wherein B is optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy.

11. The compound of claim 9, wherein B is optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me.

12. The compound of any one of claims 9 to 11, wherein B is selected from the group consisting of:

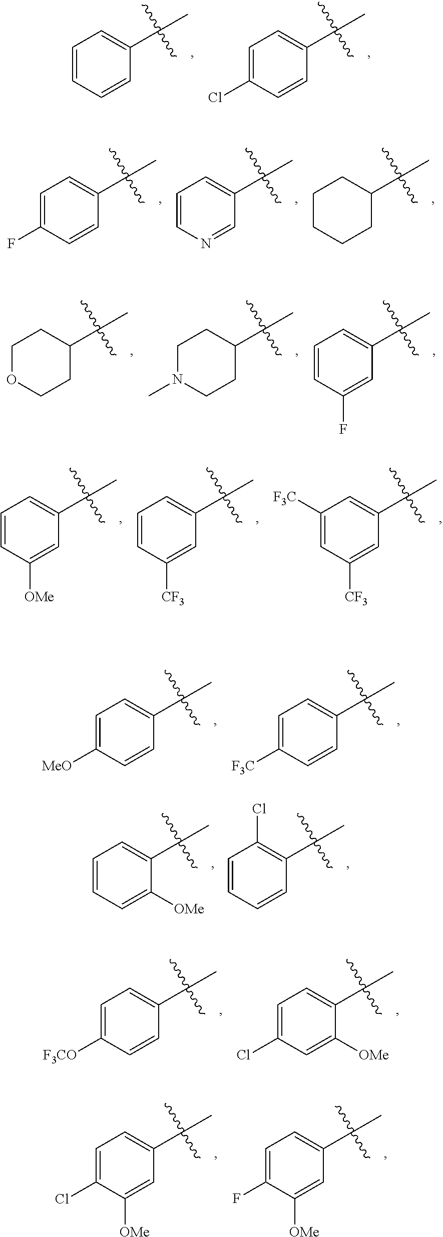

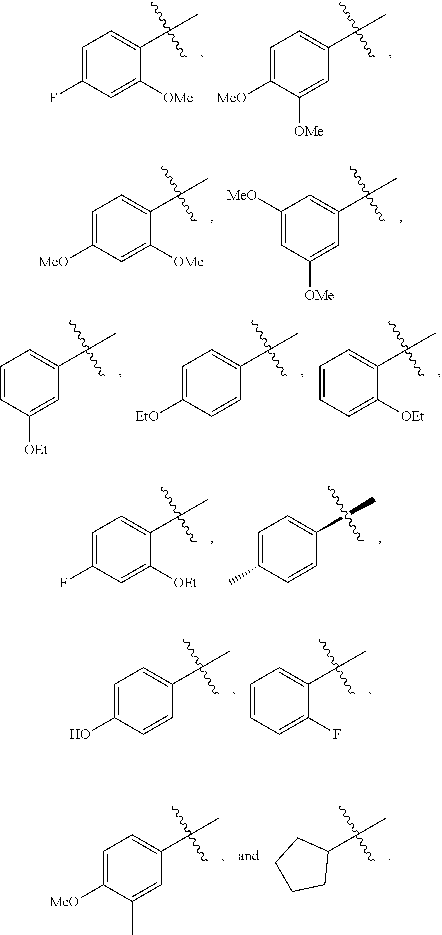

13. The compound of claim 9, wherein B is selected from the group consisting of:

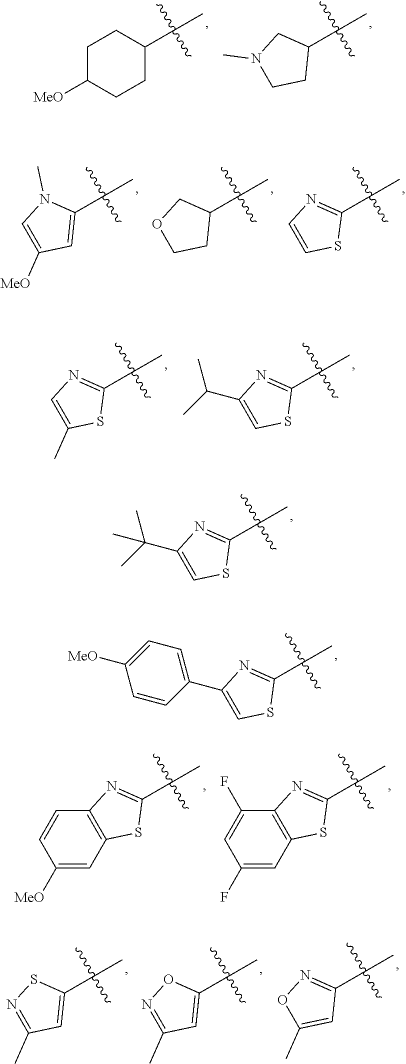

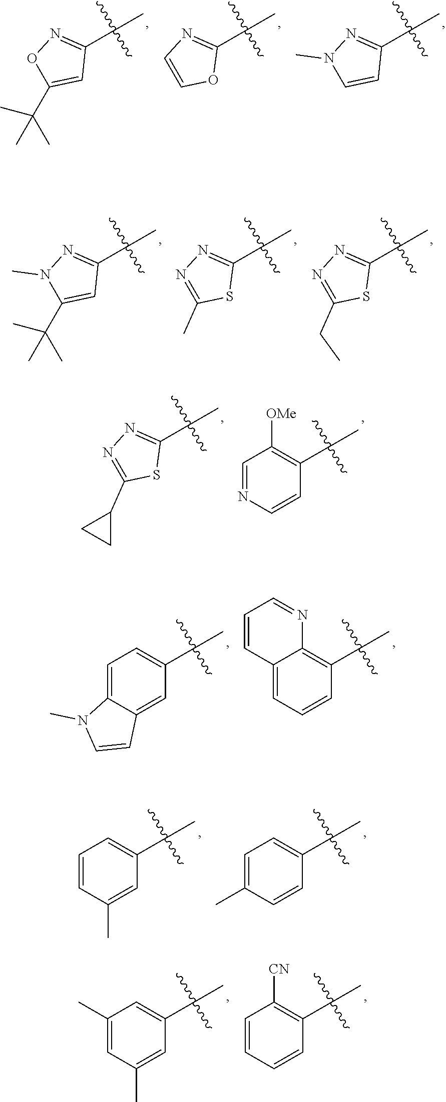

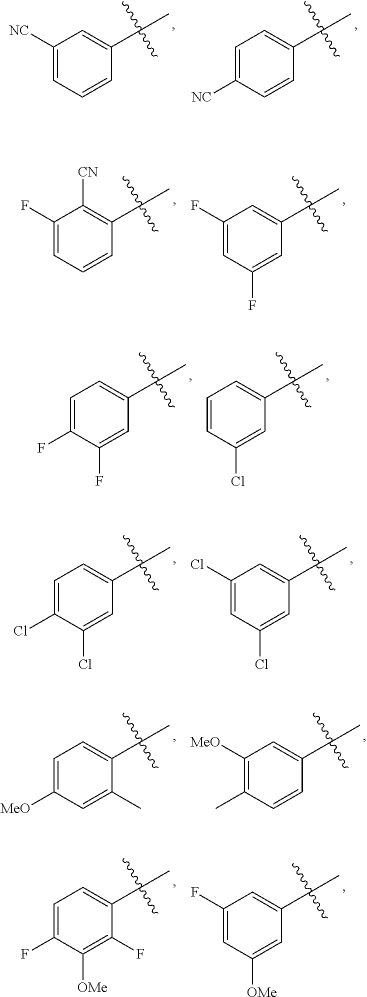

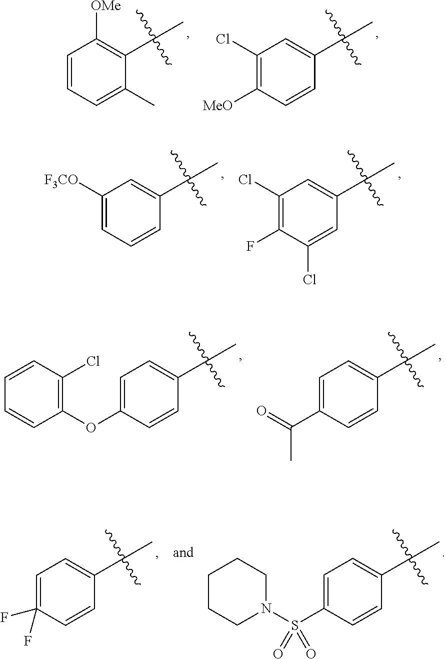

14. The compound of claim 1, selected from the group consisting of:

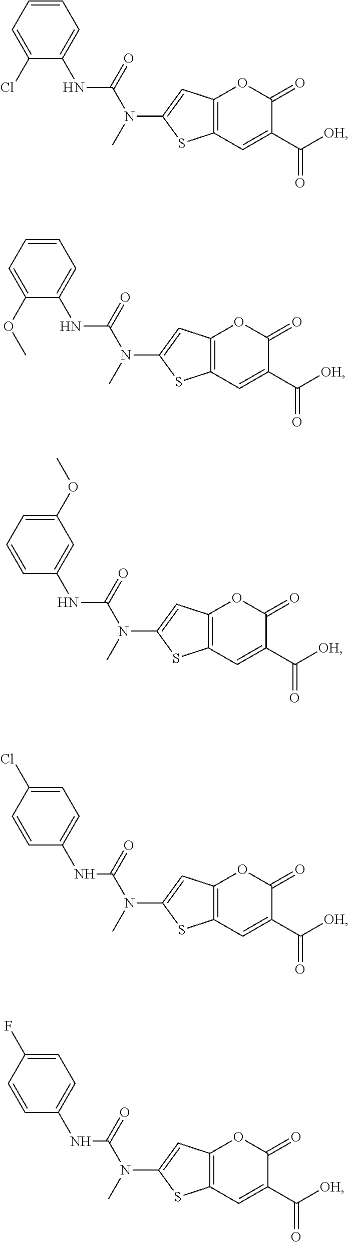 [SMILES: CN(C(=O)Nc1ccccc1Cl)c1cc2oc(=O)c(C(=O)O)cc2s1 ; COc1ccccc1NC(=O)N(C)c1cc2oc(=O)c(C(=O)O)cc2s1 ; COc1cccc(NC(=O)N(C)c2cc3oc(=O)c(C(=O)O)cc3s2)c1 ; CN(C(=O)Nc1ccc(Cl)cc1)c1cc2oc(=O)c(C(=O)O)cc2s1 ; CN(C(=O)Nc1ccc(F)cc1)c1cc2oc(=O)c(C(=O)O)cc2s1]

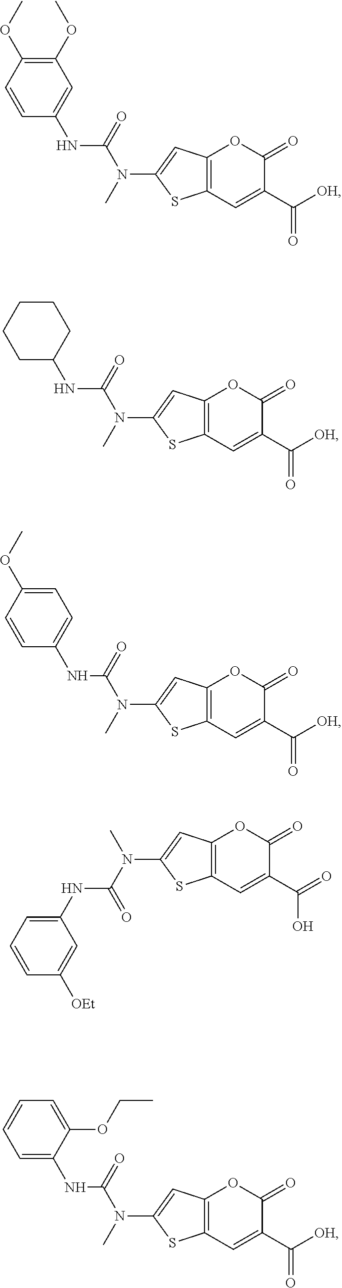 [SMILES: COc1ccc(NC(=O)N(C)c2cc3oc(=O)c(C(=O)O)cc3s2)cc1OC ; CN(C(=O)NC1CCCCC1)c1cc2oc(=O)c(C(=O)O)cc2s1 ; COc1ccc(NC(=O)N(C)c2cc3oc(=O)c(C(=O)O)cc3s2)cc1 ; CCOc1cccc(NC(=O)N(C)c2cc3oc(=O)c(C(=O)O)cc3s2)c1 ; CCOc1ccccc1NC(=O)N(C)c1cc2oc(=O)c(C(=O)O)cc2s1]

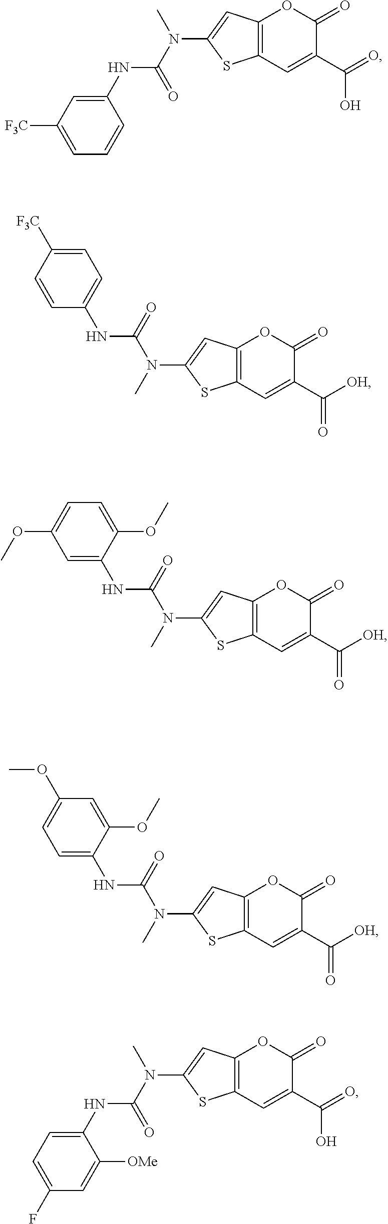 [SMILES: CN(C(=O)Nc1cccc(C(F)(F)F)c1)c1cc2oc(=O)c(C(=O)O)cc2s1 ; CN(C(=O)Nc1ccc(C(F)(F)F)cc1)c1cc2oc(=O)c(C(=O)O)cc2s1 ; COc1ccc(OC)c(NC(=O)N(C)c2cc3oc(=O)c(C(=O)O)cc3s2)c1 ; COc1ccc(NC(=O)N(C)c2cc3oc(=O)c(C(=O)O)cc3s2)c(OC)c1 ; COc1cc(F)ccc1NC(=O)N(C)c1cc2oc(=O)c(C(=O)O)cc2s1]

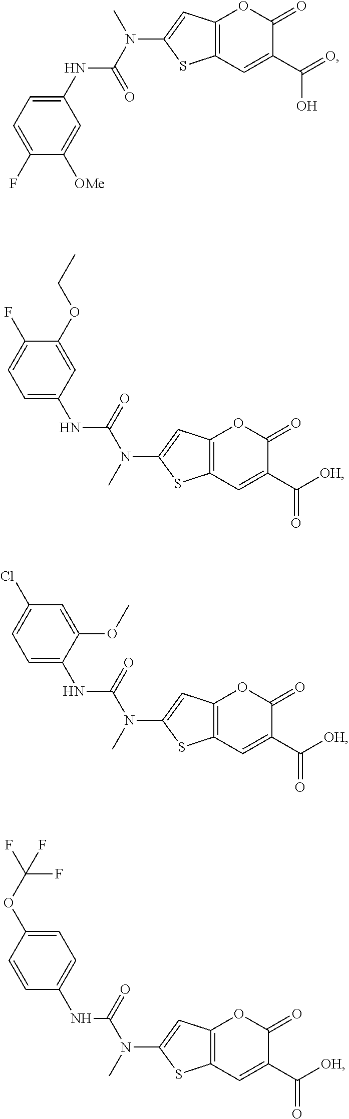 [SMILES: COc1cc(NC(=O)N(C)c2cc3oc(=O)c(C(=O)O)cc3s2)ccc1F ; CCOc1cc(NC(=O)N(C)c2cc3oc(=O)c(C(=O)O)cc3s2)ccc1F ; COc1cc(Cl)ccc1NC(=O)N(C)c1cc2oc(=O)c(C(=O)O)cc2s1 ; CN(C(=O)Nc1ccc(OC(F)(F)F)cc1)c1cc2oc(=O)c(C(=O)O)cc2s1]

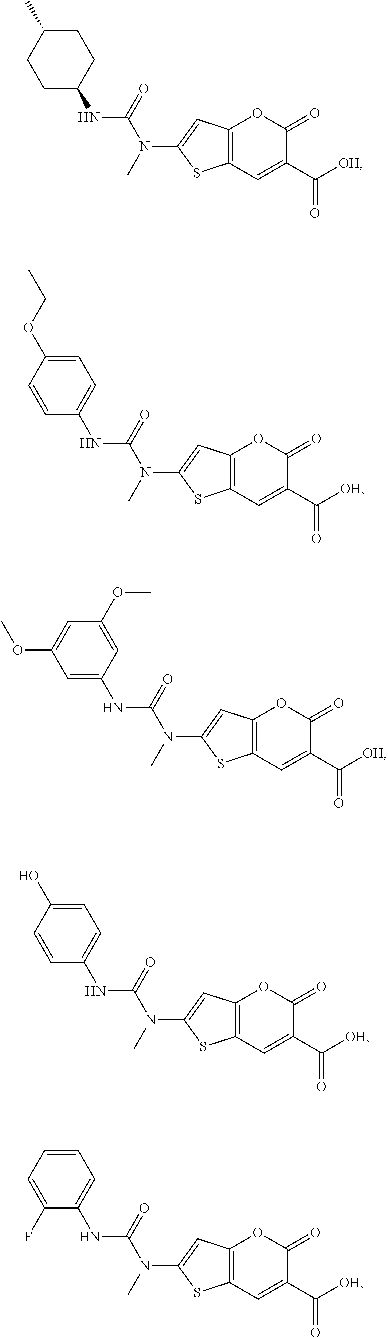 [SMILES: CN(C(=O)N[C@H]1CC[C@H](C)CC1)c1cc2oc(=O)c(C(=O)O)cc2s1 ; CCOc1ccc(NC(=O)N(C)c2cc3oc(=O)c(C(=O)O)cc3s2)cc1 ; COc1cc(NC(=O)N(C)c2cc3oc(=O)c(C(=O)O)cc3s2)cc(OC)c1 ; CN(C(=O)Nc1ccc(O)cc1)c1cc2oc(=O)c(C(=O)O)cc2s1 ; CN(C(=O)Nc1ccccc1F)c1cc2oc(=O)c(C(=O)O)cc2s1]

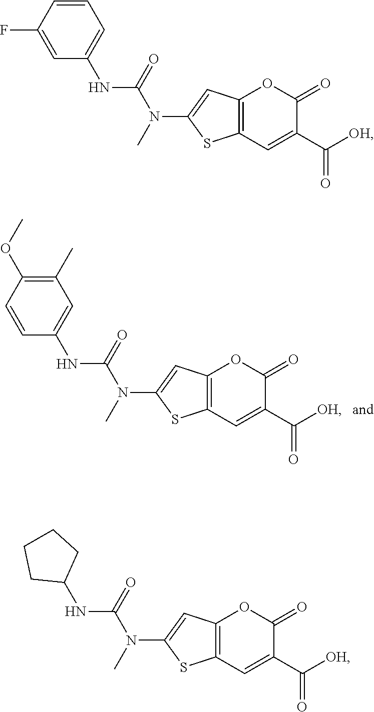 [SMILES: CN(C(=O)Nc1cccc(F)c1)c1cc2oc(=O)c(C(=O)O)cc2s1 ; COc1ccc(NC(=O)N(C)c2cc3oc(=O)c(C(=O)O)cc3s2)cc1C ; "and" ; CN(C(=O)NC1CCCC1)c1cc2oc(=O)c(C(=O)O)cc2s1]

15. The compound of claim 1, selected from the group consisting of:

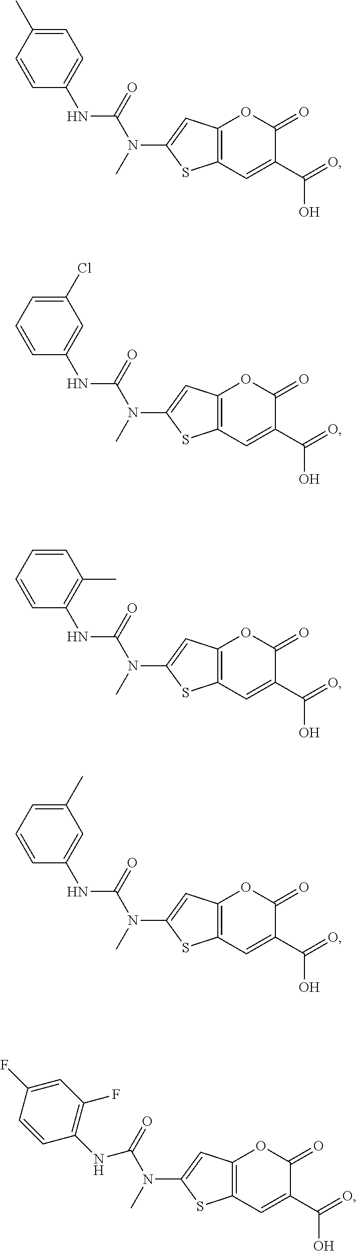 [SMILES: Cc1ccc(NC(=O)N(C)c2cc3oc(=O)c(C(=O)O)cc3s2)cc1 ; CN(C(=O)Nc1cccc(Cl)c1)c1cc2oc(=O)c(C(=O)O)cc2s1 ; Cc1ccccc1NC(=O)N(C)c1cc2oc(=O)c(C(=O)O)cc2s1 ; Cc1cccc(NC(=O)N(C)c2cc3oc(=O)c(C(=O)O)cc3s2)c1 ; CN(C(=O)Nc1ccc(F)cc1F)c1cc2oc(=O)c(C(=O)O)cc2s1]

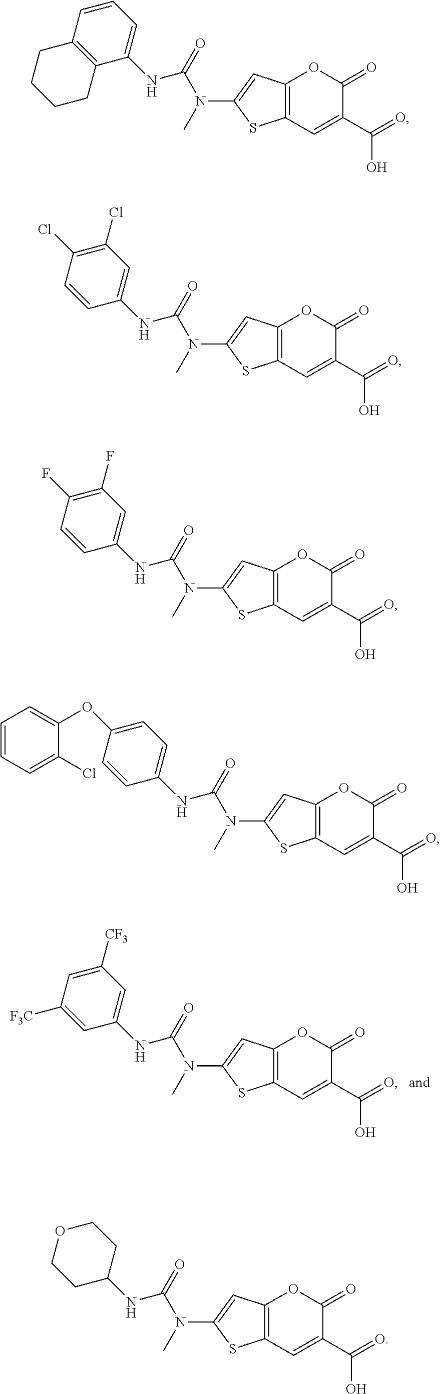

16. A compound, selected from the group consisting of: 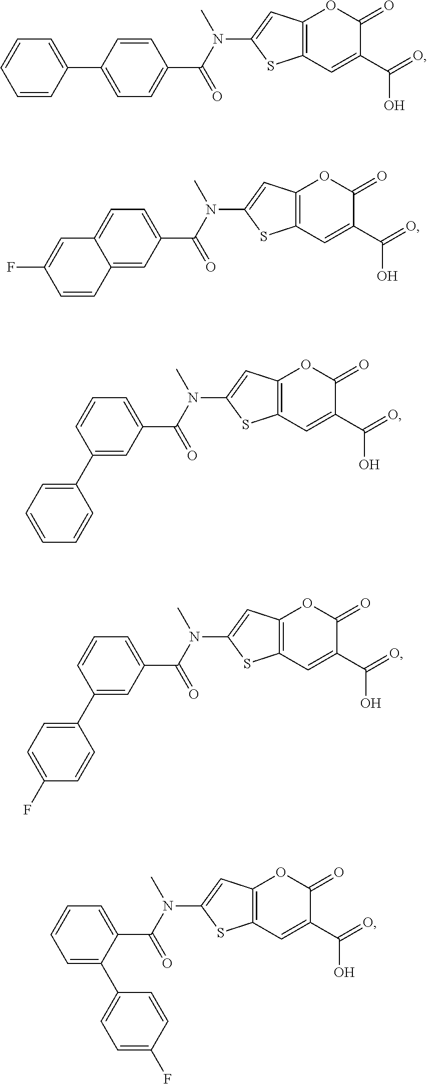 [SMILES: CN(C(=O)c1ccc(-c2ccccc2)cc1)c1cc2oc(=O)c(C(=O)O)cc2s1 ; CN(C(=O)c1ccc2cc(F)ccc2c1)c1cc2oc(=O)c(C(=O)O)cc2s1 ; CN(C(=O)c1cccc(-c2ccccc2)c1)c1cc2oc(=O)c(C(=O)O)cc2s1 ; CN(C(=O)c1cccc(-c2ccc(F)cc2)c1)c1cc2oc(=O)c(C(=O)O)cc2s1 ; CN(C(=O)c1ccccc1-c1ccc(F)cc1)c1cc2oc(=O)c(C(=O)O)cc2s1] 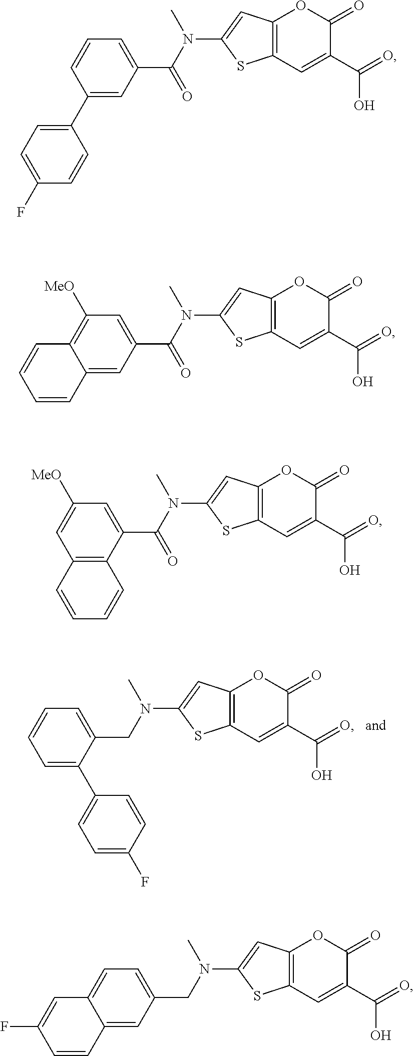 [SMILES: CN(C(=O)c1cccc(-c2ccc(F)cc2)c1)c1cc2oc(=O)c(C(=O)O)cc2s1 ; COc1cc(C(=O)N(C)c2cc3oc(=O)c(C(=O)O)cc3s2)cc2ccccc12 ; COc1cc(C(=O)N(C)c2cc3oc(=O)c(C(=O)O)cc3s2)c2ccccc2c1 ; CN(Cc1ccccc1-c1ccc(F)cc1)c1cc2oc(=O)c(C(=O)O)cc2s1 ; "and" ; CN(Cc1ccc2cc(F)ccc2c1)c1cc2oc(=O)c(C(=O)O)cc2s1]
or a pharmaceutically acceptable salt thereof.

17. A compound, selected from the group consisting of: 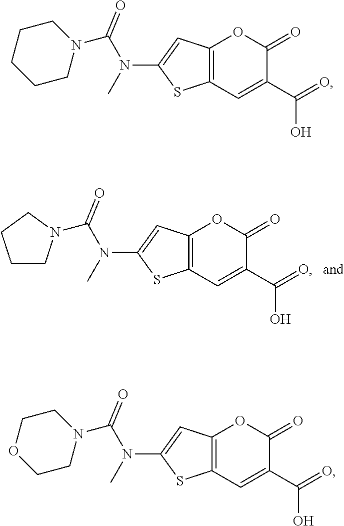 [SMILES: CN(C(=O)N1CCCCC1)c1cc2oc(=O)c(C(=O)O)cc2s1 ; CN(C(=O)N1CCCC1)c1cc2oc(=O)c(C(=O)O)cc2s1 ; CN(C(=O)N1CCOCC1)c1cc2oc(=O)c(C(=O)O)cc2s1]
or a pharmaceutically acceptable salt thereof.

18. A pharmaceutical composition comprising a compound of any one of claims 1 to 17, and a pharmaceutically acceptable carrier.

19. A method for modulating monocarboxylate transport comprising contacting a monocarboxylate transport protein with a therapeutically effective amount of a compound according to any one of claims 1 to 17.

20. A method for treating a disorder associated with monocarboxylate transport comprising administering a therapeutically effective amount of a compound according to any one of claims 1 to 17.

21. The method of claim 20, wherein the disorder is selected from the group consisting of cancer, neoplastic disorders, disorders of abnormal tissue growth, disorders of immune system, and tissue and organ rejection.

22. A process for preparing a compound of formula (VIII): 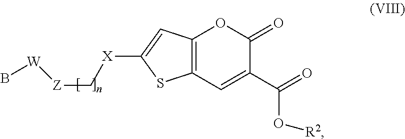
or a pharmaceutically acceptable salt thereof, comprising:
a) providing a compound of formula (IX): 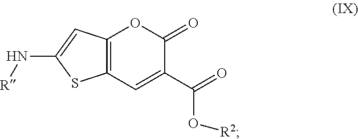
and
b) reacting with a compound of formula (X) or (XI): 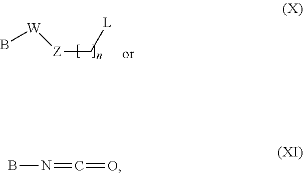
wherein L is H, OH, or halogen; and subscript n, B, W, Z, X, R², and R″ are defined according to claim 1.

23. A process for preparing a compound of formula (VIII): 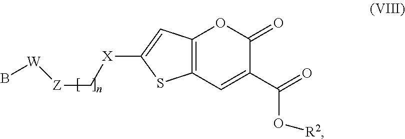
or a pharmaceutically acceptable salt thereof, comprising:
a) providing a compound of formula (IX): 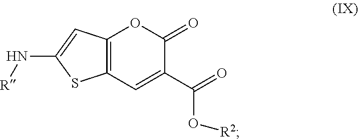
and
b) reacting with thiophosgene and an aniline of formula B—NH₂,
wherein subscript n, B, W, Z, X, R², and R″ are defined according to claim 1.

24. The process of claim 22 or 23, wherein, when R″ is hydrogen in formula (IX), the process further comprising reacting a compound of formula (XIII): 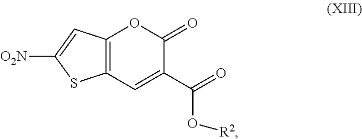
with a reducing agent to provide a compound of formula (XII): 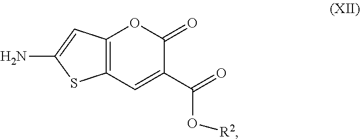
or a salt thereof, wherein R² is defined according to claim 1.

25. The process of claim 24, further comprising reacting a compound of formula (XIV): 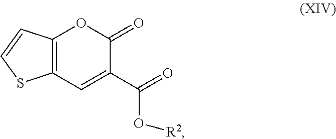
with a nitration agent to provide the compound of formula (XIII).

26. The process of claim 25, further comprising reacting a compound of formula (XV) with a compound of formula (XVI): 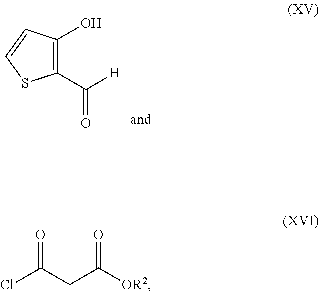
to provide the compound of formula (XIV).
